(19) United States
(12) Patent Application Publication
(10) Pub. No.: US 2019/0160058 A1
(43) Pub. Date: May 30, 2019

(54) METHODS OF TREATMENT WITH SELECTIVE CB2 RECEPTOR AGONISTS

(71) Applicant: Arena Pharmaceuticals, Inc., San Diego, CA (US)

(72) Inventor: William Shanahan, Del Mar, CA (US)

(21) Appl. No.: 16/092,259

(22) PCT Filed: Apr. 10, 2017
(86) PCT No.: PCT/US2017/026848

§ 371 Date: Oct. 9, 2018

Related U.S. Application Data
(60) Provisional application No. 62/320,572, filed on Apr. 10, 2016.

(51) Int. Cl. A61K 31/497; A61P 29/00

(52) U.S. Cl. CPC A61K 31/497; A61P 29/00

(57) ABSTRACT

Methods for treating a CB₂ receptor-related disorder (e.g., pain, fibrosis) are provided. These methods are directed to reducing the risk of adverse events based on reduced blood pressure and/or heart rate in subjects in need of treatment with a CB₂ receptor agonist compound (e.g., APD371).

### CROSS-REFERENCE TO RELATED APPLICATIONS

This application claims the benefit of U.S. Provisional Application No. 62/320,572, filed Apr. 10, 2016, the contents of which application is incorporated herein by reference in its entirety.

### FIELD OF THE INVENTION

The present application relates generally to methods of treating a human subject in need of treatment with a selective CB₂ receptor agonist.

### BACKGROUND

Cannabinoids are a group of extracellular signaling molecules that are found in both plants and animals. Signals from these molecules are mediated in animals by two G-protein coupled receptors, Cannabinoid Receptor 1 (CB₁) and Cannabinoid Receptor 2 (CB₂). CB₁ is expressed most abundantly in the neurons of the central nervous system (CNS), but is also present at lower concentrations in a variety of peripheral tissues and cells (Nature, 346:561-564, 1990), whereas CB₂ is expressed predominantly, although not exclusively, in non-neural tissues, e.g., in hematopoietic cells, endothelial cells, osteoblasts, osteoclasts, the endocrine pancreas, and cancerous cell lines (Nature, 365:61-65, 1993; and as reviewed in Pharmacol. Rev., 58(3): 389-462, 2006). CB₁ is believed to be primarily responsible for mediating the psychotropic effects of cannabinoids on the body, whereas CB₂ is believed to be primarily responsible for most of their non-neural effects.

CB₂ receptor agonists are useful in the treatment of several conditions, including pain. There is a need in the art for developing methods of using CB₂ receptor agonists in safe and effective therapies and to reduce the risk of adverse events. The methods described herein satisfy this need and provide related advantages as well.

### SUMMARY

This disclosure relates, in part, to methods of treating a human subject in need of treatment with a CB₂ receptor agonist compound (e.g., APD371).

Most CB₂ agonists in the literature have been poorly characterized in vitro. Further, many of the compounds that have been characterized are partial agonists with limited selectivity for the CB₂ receptor. For example, many CB₂ receptor agonists are promiscuous at concentrations >1 μM and produce effects that are not CB₂-mediated. Because CB₁-mediated side effects occur at a low receptor occupancy, these compounds can demonstrate unintended CB₁-mediated side effects that are misinterpreted as being CB₂-mediated.

In rodent pain models, partial CB₂ agonists (particularly those that also cause only partial internalization of the CB₂ receptor into cells) rapidly tachyphylax despite sustained plasma exposures. In contrast, full CB₂ receptor agonists demonstrate sustained efficacy that closely matches plasma exposures. As such, robust and efficient CB₂ receptor internalization is an important factor in selecting compounds for the treatment of CB₂ receptor-mediated disorders.

APD371 is a highly selective and potent agonist of the CB₂ receptor with high peripheral restriction. APD371 functions as a full CB₂ receptor agonist, and induces a high level of internalization of the CB₂ receptor into cells. These are important characteristics for reducing the incidence of safety and tolerability issues that are associated with existing therapies.

Cardiovascular effects were not seen in preclinical animal model studies for APD371. Further, an increase in heart rate was seen at the highest doses in a single ascending dose clinical trial in human subjects administered APD371. These observations make the discoveries presented herein surprising and unexpected—particularly the finding that the administration of a selective CB₂ receptor agonist compound can lead to a reduction in heart rate and/or blood pressure (compared with heart rate and/or blood pressure prior to administration of the selective CB₂ receptor agonist compound).

Although a decrease in heart rate and/or blood pressure can be asymptomatic in a healthy individual, it can present a serious risk for unhealthy individuals or in particular treatment modalities. For example, a decrease in heart rate and/or blood pressure can be problematic for an elderly individual, an individual with a preexisting cardiovascular condition (e.g., hypotension or bradycardia), an individual who is a poor metabolizer of APD371, or an individual who is receiving another therapy that decreases heart rate and/or blood pressure.

The surprising findings presented herein are taken into account in the methods of this disclosure, including methods of selecting individuals for treatment with a selective CB₂ receptor agonist compound (e.g., APD371) and in methods for treating a subject in need of treatment with a selective CB₂ receptor agonist compound. In addition, methods are provided for modifying treatment regimens for an individual who is already being treated with a selective CB₂ receptor agonist compound (e.g., APD371) and who has a risk of developing, or has developed, a low heart rate and/or low blood pressure after the initial treatment with the selective CB₂ receptor agonist compound. The modified treatment regimens include interrupting treatment with the selective CB₂ receptor agonist compound and/or continuing administering to the subject a treatment with a reduced dose of the selective CB₂ receptor agonist compared with the initial dose administered during therapy. The methods of this disclosure also encompass evaluating blood pressure and/or heart rate during the course of the treatment regimen with the selective CB₂ receptor agonist compound. Furthermore, the methods take into account drug interactions with the selective CB₂ receptor agonist compound (i.e., whether a drug being administered, or considered for administration, to a subject during therapy with the selective CB₂ receptor agonist compound reduces heart rate and/or blood pressure in the presence of the selective CB₂ receptor agonist compound).

In one exemplary embodiment, the present disclosure provides a method (Method 1) of treating a human subject in need of treatment with a selective CB₂ receptor agonist, the method comprising the steps of:
a) detecting the presence or absence of a risk factor in the human subject, wherein the risk factor is one or both of:
   i. a low heart rate and/or low blood pressure; and
   ii. the risk of a low heart rate and/or low blood pressure;
and
b1) administering a therapeutically effective amount of the selective CB₂ receptor agonist to the human subject if the risk factor condition of step a) is absent; or
b2) if the risk factor of step a) is detected, then either:
   i. not administering the selective CB₂ receptor agonist to the human subject; or
   ii. administering the selective CB₂ receptor agonist to the human subject at a dose lower than the therapeutically effective amount of step b1).

The present disclosure provides additional exemplary embodiments, including:
1.1 Method 1, wherein the risk factor of step a) is absent;
1.2 Method 1.1, further comprising administering to the human subject a further dose of the selective CB2 receptor agonist, wherein the amount of the further dose is the same or greater than the therapeutically effective amount of the selective CB2 receptor agonist of step b1);
1.3 Method 1.1, further comprising administering to the human subject a further dose of the selective CB2 receptor agonist, wherein the amount of the further dose is the same as the therapeutically effective amount of the selective CB2 receptor agonist of step b1).
1.4 Method 1, wherein the risk factor of step a) is detected;
1.5 Method 1.4, wherein the selective CB2 receptor agonist is administered to the human subject at a dose lower than the therapeutically effective amount of step b1);
1.6 Method 1.5, further comprising administering to the human subject one or more further doses of the selective CB2 receptor agonist, wherein the one or more further doses are at an amount of selective CB2 receptor agonist that is less than the therapeutically effective amount of step b1), and greater than the lower dose of Method 1.5;
1.7 Method 1.6, wherein the one or more further doses of the selective CB2 receptor agonist are of progressively increasing amounts of the selective CB2 receptor agonist;
1.8 Method 1.5, further comprising increasing the dosage amount of the selective CB2 receptor agonist;
1.9 Any Method 1 or 1.1-1.5, wherein the risk of a low heart rate and/or low blood pressure of step a) is one or more of the conditions listed in paragraphs a-f below:
   a. the subject:
      i. has had or is taking prolonged bed rest;
      ii. is within the first 24 weeks of pregnancy;
      iii. has suffered decreases in blood volume (e.g., as a result of trauma, severe internal bleeding, dehydration);
      iv. is taking an anti-hypertensive medication, a diuretic, a beta-blocker, a drug for Parkinson's disease, a tricyclic antidepressant, an erectile dysfunction drug alone or in combination with nitroglycerine, digoxin, or an antiarrhythmic;
      v. is taking narcotics or alcohol;
      vi. has had a heart attack;
      vii. has problems with heart valve(s);
      viii. has coronary artery disease;
      ix. has endocarditis, myocarditis, hypothyroidism, parathyroid disease, Addison's disease, low blood sugar, diabetes, septic shock, neutrally mediated hypotension, anemia, an electrolyte imbalance, high levels of potassium in the blood, or a deficiency in vitamin B-12 and/or folic acid;
   b. prior administration of nitrates, alpha blockers, beta blockers, anti-hypertensive drugs, vasodilators, digoxin, amiodarone, alcohol, or medications that are inhibitors or inducers of CYP, for example CYP1A2, CYP2B6, CYP2C8, CYP2C9, CYP2C19, CYP2D6, CYP2E1, CYP3A or CYP3A4/3A5;
   c. prior administration of one or more:
      i. agents that increase the exposure of the selective CB2 receptor agonist;
      ii. agents that slow the metabolism of the selective CB2 receptor agonist;
      iii. agents that increase the accumulation of metabolites of the selective CB2 receptor agonist in the human subject compared to the absence of the drug or agent;
      iv. agents that compete with the selective CB2 receptor agonist for protein binding; or
      v. agents that cause QT prolongation;
   d. a medical condition that is known to decrease heart rate and/or blood pressure, for example a heart condition that leads to low heart rate (bradycardia);
   e. a history of cerebrovascular disease, dizziness, lightheadedness, fainting, headache, nausea, hypotension, syncope, shock, hemodynamic instability, bradycardia, aortic stenosis, myocardial infarction, ischemia, heart failure, or a conduction abnormality; and
   f. impaired function of a CYP, or being a poor or intermediate CYP metabolizer, for example where the CYP is CYP1A2, CYP2B6, CYP2C8, CYP2C9, CYP2C19, CYP2D6, CYP2E1, CYP3A or CYP3A4/3A5.
1.10 Any Method 1 or 1.1-1.6, wherein the risk factor of step a) is a low heart rate and/or low blood pressure.
1.11 Any Method 1 or 1.1-1.6, wherein the risk factor of step a) is a the risk of a low heart rate and/or low blood pressure.
1.12 Any Method 1 or 1.1-1.8, further comprising the step of identifying a human subject in need of treatment with a selective CB2 receptor agonist.

It will be appreciated that the listings in paragraphs a-f in Method 1.6 above are not mutually exclusive, and can overlap such that a given condition may be within more than one of the paragraphs a-f. For example, an inhibitor of CYP in paragraph b can also be an agent that increases the exposure of the selective CB2 receptor agonist of paragraph c.

In a second exemplary embodiment, the disclosure provides a method (Method 2) of treating a human subject in need of treatment with a selective CB2 receptor agonist, the method comprising the steps of:
a. administering a therapeutically effective amount of the selective CB2 receptor agonist to the human subject;
b. detecting the heart rate and/or blood pressure of the human subject after administration of the selective CB2 receptor agonist; and either:
   i. continuing administration of the selective CB2 receptor agonist to the human subject if the heart rate and/or blood pressure of the human subject after administration of the selective CB2 receptor agonist is not decreased by a defined amount compared to the heart rate and/or blood pressure prior to administration of the selective CB2 receptor agonist; or
   ii. discontinuing the administration of the therapeutically effective amount of the selective CB2 receptor agonist if the heart rate and/or blood pressure of the human subject after administration of the selective CB2 receptor agonist is decreased by a defined amount compared to the heart rate and/or blood pressure prior to administration of the selective CB2 receptor agonist.
The present disclosure provides additional exemplary embodiments, including:
2.1 Method 2, wherein the heart rate and/or blood pressure of the subject after administration of the selective CB2 receptor agonist is decreased by a defined amount compared to the heart rate and/or blood pressure prior to administration of the selective CB2 receptor agonist;
   the method further comprising administering to the human subject a lower dose of the selective CB2 receptor agonist;
2.2 Method 2.1, wherein the lower dose selective CB2 receptor agonist is less than the therapeutically effective amount of the selective CB2 receptor agonist of step (a).
2.3 Method 2.2, further comprising administering to the human subject one or more further doses of the selective CB2 receptor agonist, wherein the one or more further doses are at an amount of selective CB2 receptor agonist that is less than the therapeutically effective amount of step b1), and greater than the lower dose of Method 2.2;
2.4 Method 2.3, wherein the one or more further doses of the selective CB2 receptor agonist are of progressively increasing amounts of the selective CB2 receptor agonist;
2.5 Method 2.1, further comprising increasing the dosage amount of the selective CB2 receptor agonist;
2.6 Method 2, wherein the heart rate and/or blood pressure of the subject after administration of the selective CB2 receptor agonist is not decreased by a defined amount compared to the heart rate and/or blood pressure prior to administration of the selective CB2 receptor agonist;
   the method further comprising administering to the human subject a further dose of the selective CB2 receptor agonist that is the same or greater than the therapeutically effective amount of the selective CB2 receptor agonist of step a);
2.7 Method 2.6, wherein the further dose of the selective CB2 receptor agonist is the same as the therapeutically effective amount of the selective CB2 receptor agonist of step a);
2.8 Method 2.6, wherein the further dose of the selective CB2 receptor agonist is greater than the therapeutically effective amount of the selective CB2 receptor agonist of step a);
2.9 Any Method 2 or 2.1-2.8, further comprising the step of identifying a human subject in need of treatment with a selective CB2 receptor agonist;

In a third exemplary embodiment, the disclosure provides a method (Method 3) of treating a human subject in need of treatment with a selective CB2 receptor agonist, the method comprising the steps of:
a) detecting the heart rate and/or blood pressure of the human subject prior to administration of a selective CB2 receptor agonist;
b) administering a therapeutically effective amount of the selective CB2 receptor agonist to the human subject;
c) detecting the heart rate and/or blood pressure of the human subject after administration of the selective CB2 receptor agonist; and either:
   i) if the heart rate and/or blood pressure detected in step c) are not decreased by a defined amount compared to the heart rate and/or blood pressure prior to administration of the selective CB2 receptor agonist, then continuing administering the selective CB2 receptor agonist to the human subject; or
   ii) if the heart rate and/or blood pressure detected in step c) is decreased by a defined amount compared to the heart rate and/or blood pressure prior to administration of the selective CB2 receptor agonist, then either:
      discontinuing administering of the selective CB2 receptor agonist to the human subject; or
      continuing administering of the selective CB2 receptor agonist to the human subject at a dose lower than the dose of the selective CB2 receptor agonist of step b);

The present disclosure provides additional exemplary embodiments, including:
3.1 Method 3, wherein the heart rate and/or blood pressure detected in step c) are not decreased by a defined amount compared to the heart rate and/or blood pressure prior to administration of the selective CB2 receptor agonist;
3.2 Method 3, wherein the heart rate and/or blood pressure detected in step c) is decreased by a defined amount compared to the heart rate and/or blood pressure prior to administration of the selective CB2 receptor agonist;
3.3 Method 3.2, wherein administration of the selective CB2 receptor agonist to the human subject is continued at a dose lower than the dose of the selective CB2 receptor agonist of step b);
3.4 Method 3.3, further comprising administering to the human subject one or more further doses of the selective CB2 receptor agonist, wherein the one or more further doses are at an amount of selective CB2 receptor agonist that is less than the therapeutically effective amount of step b), and greater than the lower dose of step (c) ii;
3.5 Method 3.4, wherein the one or more further doses of the selective CB2 receptor agonist are of progressively increasing amounts of the selective CB2 receptor agonist;
3.6 Method 3.2, further comprising increasing the dosage amount of the selective CB2 receptor agonist;
3.7 Any Method 3 or 3.1-3.6, wherein the dose lower than the dose of the selective CB2 receptor agonist of step b) is:
   a dose that is from, or from about, 10% to 80% of the therapeutically effective amount of the selective CB2 receptor agonist of step b); for example 80%, 75%, 70%, 65%, 60%, 55%, 50%, 45%, 40%, 35%, 30%, 25%, 20%, 15%, or 10% of the therapeutically effective amount of the selective CB2 receptor agonist of step b); for example 50% or 75% of the therapeutically effective amount of the selective CB2 receptor agonist of step b); or
   a dose that is selected from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 475 mg; for example 25 mg, 50 mg, 75 mg, or 100 mg three times daily; for example 75 mg, 150 mg; 225 mg or 300 mg daily;
3.8 Any Method 3 or 3.1-3.4, wherein the human subject was previously administered an agent selected from one or more of the agents listed below in paragraphs a-c:
   a. an anti-hypertensive medication, a diuretic, a beta-blocker, a drug for Parkinson's disease, a tricyclic antidepressant, an erectile dysfunction drug alone or in combination with nitroglycerine, digoxin, or an antiarrhythmics, narcotics, or alcohol;
   b. nitrates, alpha blockers, beta blockers, anti-hypertensive drugs, vasodilators, digoxin, amiodarone, alcohol, or medications that are inhibitors or inducers of CYP, for example CYP1A2, CYP2B6, CYP2C8, CYP2C9, CYP2C19, CYP2D6, CYP2E1, CYP3A or CYP3A4/3A5;
   c. agents that:
      i. increase the exposure of the selective CB2 receptor agonist;
      ii. slow the metabolism of the selective CB2 receptor agonist;
      iii. increase the accumulation of metabolites of the selective CB2 receptor agonist in the human subject compared to the absence of the drug or agent;
      iv. compete with the selective CB2 receptor agonist for protein binding; or
      v. cause QT prolongation.
3.9 Any Method 3 or 3.1-3.8, further comprising the step of identifying a human subject in need of treatment with a selective CB2 receptor agonist;

The present disclosure provides additional exemplary embodiments, including:

A1. Any Method 1 or 1.1-1.12, or Method 2 or 2.1-2.9, or Method 3 or 3.1-3.9, wherein the low heart rate is less than, or less than about, 60, 55, 50, 45, or 40 beats per minute (bpm); for example less than, or less than about, 50 bpm; and/or wherein the low heart rate is at least, or at least about, a 10 bpm reduction from the heart rate prior to administration of the selective CB2 receptor agonist;

A2. Any Method 1 or 1.1-1.12, or Method 2 or 2.1-2.9, or Method 3 or 3.1-3.9, or Method A1, wherein the low blood pressure is a systolic blood pressure of less than, or less than about, 120, 115, 110, 105, 100, 95, 90, 85, 80, 75, or 70 mmHg; for example a systolic blood pressure less than, or less than about, 90 mmHg;

A3. Any Method 1 or 1.1-1.12, or Method 2 or 2.1-2.9, or Method 3 or 3.1-3.9, or Method A1-A2, wherein the low blood pressure is a diastolic blood pressure of less than, or less than about, 80, 75, 70, 65, 60, 55, or 50 mmHg; for example a diastolic blood pressure less than, or less than about, 60 mmHg; or a diastolic blood pressure less than, or less than about, 50 mmHg;

A4. Any Method 1 or 1.1-1.12, or Method 2 or 2.1-2.9, or Method 3 or 3.1-3.9, or Method A1-A3, wherein the low blood pressure is a systolic blood pressure less than, or less than about 90 mmHg, and wherein the diastolic blood pressure less than, or less than about, 50 mmHg, or a systolic blood pressure less than, or less than about 95 mmHg, and a diastolic blood pressure less than, or less than about, 60 mmHg, and wherein the decrease by a defined amount of blood pressure decrease is at least a 10 mmHg reduction from the systolic and/or diastolic blood pressure prior to administration of the selective CB2 receptor agonist;

A5. Any Method 1 or 1.1-1.12, or Method 2 or 2.1-2.9, or Method 3 or 3.1-3.9, or Method A1-A4, wherein the decrease in heart rate by a defined amount is a decrease in heart rate of at least, or of at least about, 5%, 10%, 15%, 20%, or 25%; or of, or of about, 5, 10, 15, 20, or 25 bpm;

A6. Any Method 1 or 1.1-1.12, or Method 2 or 2.1-2.9, or Method 3 or 3.1-3.9, or Method A1-A5, wherein the decrease in blood pressure by a defined amount is a decrease from baseline systolic blood pressure of at least, or at least about, 5%, 10%, 15%, 20%, or 25%; and/or a decrease from baseline diastolic blood pressure of at least, or at least about, 5%, 10%, 15%, 20%, or 25%;

A7. Any Method 1 or 1.1-1.12, or Method 2 or 2.1-2.9, or Method 3 or 3.1-3.9, or Method A1-A6, wherein the decrease in blood pressure by a defined amount is a decrease in systolic blood pressure of at least, or of at least about, 5, 10, 15, 20, or 25 mmHg; and/or a decrease in diastolic blood pressure of at least, or of at least about, 5, 10, 15, 20, or 25 mmHg;

A8. Any Method 1 or 1.1-1.12, or Method 2 or 2.1-2.9, or Method 3 or 3.1-3.9, or Method A1-A7, wherein the selective CB2 receptor agonist is selected from Compounds A-H as described below;

A9. Any Method 1 or 1.1-1.12, or Method 2 or 2.1-2.9, or Method 3 or 3.1-3.9, or Method A1-A8, wherein the selective CB2 receptor agonist is APD371;

A10. Any Method 1 or 1.1-1.12, or Method 2 or 2.1-2.9, or Method 3 or 3.1-3.9, or Method A1-A9, wherein the selective CB2 receptor agonist is APD371, and wherein the therapeutically effective amount (first dosage amount) is selected from, or from about: 10 mg to 500 mg; for example 10 mg to 250 mg; for example 10 mg to 150 mg; for example 25 mg to 100 mg; for example 25 mg to 250 mg, for example 25 mg, 50 mg, 75 mg, 100 mg, 200 mg, or 250 mg; for example 25 mg, 50 mg, 75 mg or 100 mg;

A11. Any Method 1 or 1.1-1.12, or Method 2 or 2.1-2.9, or Method 3 or 3.1-3.9, or Method A1-A10, wherein the therapeutically effective amount (first dosage amount) of the selective CB2 receptor agonist is administered more than once; for example at a frequency of: once a day, twice a day, three times a day, or four times a day;

A12. Any Method 1 or 1.1-1.12, or Method 2 or 2.1-2.9, or Method 3 or 3.1-3.9, or Method A1-A11, wherein the selective CB2 receptor agonist is APD371, and wherein the second dosage amount is selected from, or from about: 10 mg to 500 mg; for example 10 mg to 250 mg; for example 10 mg to 150 mg; for example 25 mg to 100 mg; for example 25 mg to 250 mg; for example 25 mg, 50 mg, 75 mg, 100 mg, 200 mg, or 250 mg; for example 25 mg, 50 mg, 75 mg or 100 mg;

A13. Any Method 1 or 1.1-1.12, or Method 2 or 2.1-2.9, or Method 3 or 3.1-3.9, or Method A1-A12, wherein the second dosage amount of the selective CB2 receptor agonist is administered more than once; for example at a frequency selected of: once a day, twice a day, three times a day, or four times a day;

A14. Any Method 1.2, 1.4, 1.5-1.9, or Method 2 or 2.1-2.2, or Method 3 or 3.1-3.2, or Method A1-A13, further comprising evaluating the heart rate and/or blood pressure of the human subject following administration of the second dosage amount;

A15. Any Method 1 or 1.1-1.12, or Method 2 or 2.1-2.9, or Method 3 or 3.1-3.9, or Method A1-A14, wherein the selective CB2 receptor agonist is APD371, and wherein the standard dose is selected from, or from about: 10 mg to 500 mg; for example 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg.

A16. Method 1 or 1.1-1.12, or Method 2 or 2.1-2.9, or Method 3 or 3.1-3.9, or Method A1-A15, wherein the selective CB2 receptor agonist is APD371, and wherein the standard dose is selected from, or from about: 10 mg to 250 mg; for example 10 mg to 150 mg; for example 25 mg to 100 mg; for example 25 mg, 50 mg, 75 mg, 100 mg, 200 mg, or 250 mg; for example 25 mg, 50 mg, 75 mg or 100 mg.

A17. Any Method 1 or 1.1-1.12, or Method 2 or 2.1-2.9, or Method 3 or 3.1-3.9, or Method A1-A16, wherein the standard dose is administered at a frequency of: once a day, twice a day, three times a day, or four times a day;

A18. Any Method 1 or 1.1-1.12, or Method 2 or 2.1-2.9, or Method 3 or 3.1-3.9, or Method A1-A17, wherein the selective CB2 receptor agonist is APD371, and wherein the therapeutically effective amount of the selective CB2 receptor agonist is a dose of, or about, 25 mg to 100 mg; A19. Any Method 1 or 1.1-1.12, or Method 2 or 2.1-2.9, or Method 3 or 3.1-3.9, or

Method A1-A18, wherein the therapeutically effective amount of the selective CB2 receptor agonist is from 25 mg to 100 mg per administration, administered twice or three times daily; or is less than about 600, about 400, about 300, or about 250 mg daily; or is about 75 mg, about 150 mg, about 225 mg, or about 300 mg daily;

A20. Any Method 1 or 1.1-1.12, or Method 2 or 2.1-2.9, or Method 3 or 3.1-3.9, or Method A1-A19, wherein the selective CB2 receptor agonist is APD371, and wherein the dose lower than the standard dose is 75 mg, 50 mg, 25 mg, or 12.5 mg;

A21. Any Method 1 or 1.1-1.12, or Method 2 or 2.1-2.9, or Method 3 or 3.1-3.9, or Method A1-A20, wherein the dose lower than the therapeutically effective amount of the selective CB2 receptor agonist is, or is about, 50 mg;

A22. Any Method 1 or 1.1-1.12, or Method 2 or 2.1-2.9, or Method 3 or 3.1-3.9, or Method A1-A21, wherein the dose lower than the therapeutically effective amount of the selective CB2 receptor is, or is about, 100 mg;

A23. Any Method 1 or 1.1-1.12, or Method 2 or 2.1-2.9, or Method 3 or 3.1-3.9, or Method A1-A22, wherein the dose lower than the therapeutically effective amount of the selective CB2 receptor is administered at a frequency selected from the group consisting of: once a day, twice a day, three times a day, and four times a day;

A24. Any Method 1 or 1.1-1.12, or Method 2 or 2.1-2.9, or Method 3 or 3.1-3.9, or Method A1-A23, further comprising evaluating the heart rate and/or blood pressure of the human subject prior to or following an administration of the selective CB2 receptor agonist.

A25. Any Method 1 or 1.1-1.12, or Method 2 or 2.1-2.9, or Method 3 or 3.1-3.9, or Method A1-A24, further comprising evaluating the heart rate and/or blood pressure of the human subject following administration of the lower dosage amount.

A26. Any Method 1 or 1.1-1.12, or Method 2 or 2.1-2.9, or Method 3 or 3.1-3.9, or Method A1-A25, further comprising monitoring the human subject for an adverse reaction (such as hypotension, syncope, and/or bradycardia) following administration of the selective CB2 receptor agonist;

A27. Any Method 1 or 1.1-1.12, or Method 2 or 2.1-2.9, or Method 3 or 3.1-3.9, or Method A1-A26, further comprising evaluating the heart rate and/or blood pressure and/or a condition related thereto for the human subject following administration of the selective CB2 receptor agonist; for example wherein evaluating the heart rate and/or blood pressure of the human subject comprises measuring the heart rate and/or blood pressure of the human subject;

A28. Any Method 1 or 1.1-1.12, or Method 2 or 2.1-2.9, or Method 3 or 3.1-3.9, or Method A1-A27, wherein the heart rate and/or blood pressure of the human subject is evaluated about, or at least about, 0.5, 1, 1.5, 2, 2.5, 3, 3.5, 4, 4.5, 5, 5.5, or 6 hours following administration of the selective CB2 receptor agonist;

A29. Any Method 1 or 1.1-1.12, or Method 2 or 2.1-2.9, or Method 3 or 3.1-3.9, or Method A1-A28, wherein a risk factor is detected or not detected; or the heart rate and/or blood pressure of the human subject is detected and/or evaluated; based on at least one determination selected from the group consisting of:
(i) determining by electrocardiogram that the human subject has or does not have a low heart rate;
(ii) determining by vital signs that the human subject has or does not have a low heart rate;
(iii) determining by vital signs that the human subject has or does not have a low systolic and/or diastolic blood pressure;
(iv) determining that the human subject has or does not have a history of low heart rate and/or low systolic and/or diastolic blood pressure and/or condition related thereto;
(v) determining that the human subject has or does not have impaired elimination of the selective CB2 receptor agonist; and
(vi) determining that the human subject is or is not a poor or intermediate CYP metabolizer;

A30. Any Method 1 or 1.1-1.12, or Method 2 or 2.1-2.9, or Method 3 or 3.1-3.9, or Method A1-A29, wherein the selective CB2 receptor agonist is APD371, wherein the therapeutically effective amount of APD371 is, or is about:
10 mg, 15 mg, 20 mg, 25 mg, 30 mg, 35 mg, 40 mg, 45 mg, 50 mg, 60 mg, 65 mg, 70 mg, 75 mg, 80 mg, 85 mg, 90 mg, 95 mg, 100 mg, 110 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg; or
is, or is about, 10 mg, 25 mg, 50 mg, 75 mg, 150 mg, or 200 mg; and is administered once, twice, three times or four times per day;

A31. Any Method 1 or 1.1-1.12, or Method 2 or 2.1-2.9, or Method 3 or 3.1-3.9, or Method A1-A30, wherein the selective CB2 receptor agonist is APD371, wherein the maximum dose of APD371 is, or is about:
10 mg, 25 mg, 50 mg, 75 mg, 100 mg, 125 mg, 150 mg, 175 mg, or 200 mg per administration; and/or
75 mg, 150 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg per day;

A32. Any Method 1 or 1.1-1.12, or Method 2 or 2.1-2.9, or Method 3 or 3.1-3.9, or Method A1-A31, wherein the selective CB2 receptor agonist is APD371, and wherein the amount of APD371 is less than or equal to 200 mg per administration;

A33. Any Method 1 or 1.1-1.12, or Method 2 or 2.1-2.9, or Method 3 or 3.1-3.9, or Method A1-A28, wherein:
the normal heart rate is at least, or at least about, 60 bpm; or is about 60 bpm to about 100 bpm; and
the normal blood pressure is a systolic blood pressure of about 90 mmHg to about 119 mmHg, and/or a diastolic blood pressure of about 60 mmHg to about 79 mmHg;

A32. Any Method 1 or 1.1-1.12, or Method 2 or 2.1-2.9, or Method 3 or 3.1-3.9, or Method A1-A28, wherein the human subject is not part of a multi-center, placebo-controlled, double-blind trial designed to:

(i) examine the safety or efficacy of the selective CB2 receptor agonist, and/or

(ii) have data therefrom submitted to a regulatory agency for approval of the selective CB2 receptor agonist for treatment of human subjects;

A34. Any Method 1 or 1.1-1.12, or Method 2 or 2.1-2.9, or Method 3 or 3.1-3.9, or Method A1-A33, wherein the human subject is elderly.

A35. Any Method 1 or 1.1-1.12, or Method 2 or 2.1-2.9, or Method 3 or 3.1-3.9, or Method A1-A34, wherein:
the heart rate is selected from the group consisting of a resting heart rate, a supine heart rate, and a standing heart rate;
the blood pressure is selected from the group consisting of systolic blood pressure and diastolic blood pressure; wherein:
   the systolic blood pressure is selected from the group consisting of a resting systolic blood pressure, a supine systolic blood pressure, and a standing systolic blood pressure; and
   the diastolic blood pressure is selected from the group consisting of a resting diastolic blood pressure, a supine diastolic blood pressure, and a standing diastolic blood pressure;

A36. Any Method 1 or 1.1-1.12, or Method 2 or 2.1-2.9, or Method 3 or 3.1-3.9, or Method A1-A35, wherein the selective CB2 receptor agonist is administered orally;

A37. Any Method 1 or 1.1-1.12, or Method 2 or 2.1-2.9, or Method 3 or 3.1-3.9, or Method A1-A36, wherein the selective CB2 receptor agonist is in the form of a tablet or capsule;

A38. Any Method 1 or 1.1-1.12, or Method 2 or 2.1-2.9, or Method 3 or 3.1-3.9, or Method A1-A37, wherein the risk factor is selected from at least one of the following: dizziness, lightheadedness, fainting, headache, nausea, hypotension, syncope, shock, hemodynamic instability, bradycardia, aortic stenosis, myocardial infarction, ischemia, heart failure, and a conduction abnormality;

A39. Any Method 1 or 1.1-1.12, or Method 2 or 2.1-2.9, or Method 3 or 3.1-3.9, or Method A1-A38, wherein the treatment is the treatment or prevention of a CB2 receptor-mediated disorder;

A40. Any Method 1 or 1.1-1.12, or Method 2 or 2.1-2.9, or Method 3 or 3.1-3.9, or Method A1-A39, wherein the treatment is the treatment or prevention of a CB2 receptor-mediated disorder selected from pain; bone pain; joint pain; muscle pain; dental pain; migraine and other headache pain; inflammatory pain; neuropathic pain; pain that occurs as an adverse effect of therapeutics; pain associated with a disorder selected from: osteoarthritis, cancer, multiple sclerosis, allergic reactions, nephritic syndrome, scleroderma, thyroiditis, diabetic neuropathy, fibromyalgia, HIV related-neuropathy, sciatica, and autoimmune conditions; acute and/or chronic inflammatory pain; acute and/or chronic neuropathic pAain; chemotherapy-induced pain; acute post-operative pain; abdominal pain associated with inflammatory bowel disease (IBD); non-radicular low back pain; pain from liver fibrosis, primary biliary cirrhosis, nonalcoholic steatohepatitis, renal fibrosis, endometriosis, and interstitial cystitis; hyperalgesia; allodynia; inflammatory hyperalgesia; neuropathic hyperalgesia; acute nociception; osteoporosis; multiple sclerosis-associated spasticity; autoimmune disorders, for example an autoimmune disorder selected from the group consisting of: multiple sclerosis, Guillan-Barré syndrome, polyradiculoneuropathy, chronic inflammatory demyelination, rheumatoid arthritis, psoriatic arthritis, ankylosing spondylarthritis, and reactive arthritis; allergic reactions, for example, an allergic reaction associated with a disorder selected from: atopic dermatitis, pruritis, urticaria, asthma, conjunctivitis, allergic rhinitis, and anaphylaxis; CNS inflammation for example, CNS inflammation associated with a disorder selected from: Alzheimer's disease, stroke, dementia, amyotrophic lateral sclerosis, and human immunodeficiency virus; atherosclerosis; undesired immune cell activity, and inflammation associated with a disorder selected from: osteoarthritis, anaphylaxis, Behcet's disease, graft rejection, vasculitis, gout, spondylitis, viral disease, bacterial disease, lupus, inflammatory bowel disease, autoimmune hepatitis, and type 1 diabetes mellitus; age-related macular degeneration; cough; leukemia; lymphoma; CNS tumors; prostate cancer; Alzheimer's disease; stroke-induced damage; dementia; amyotrophic lateral sclerosis, and Parkinson's disease.

A41. Any Method 1 or 1.1-1.12, or Method 2 or 2.1-2.9, or Method 3 or 3.1-3.9, or Method A1-A40, wherein the treatment is the treatment of acute and/or chronic inflammatory pain;

A42. Any Method 1 or 1.1-1.12, or Method 2 or 2.1-2.9, or Method 3 or 3.1-3.9, or Method A1-A41, wherein the treatment is the treatment of acute and/or chronic neuropathic pain;

A43. Any Method 1 or 1.1-1.12, or Method 2 or 2.1-2.9, or Method 3 or 3.1-3.9, or Method A1-A42, wherein the treatment is the treatment of inflammation or an inflammatory condition;

A44. Any Method 1 or 1.1-1.12, or Method 2 or 2.1-2.9, or Method 3 or 3.1-3.9, or Method A1-A43, wherein the human subject is a poor or intermediate CYP metabolizer;

A45. Any Method 1 or 1.1-1.12, or Method 2 or 2.1-2.9, or Method 3 or 3.1-3.9, or Method A1-A44, wherein the human subject is a poor or intermediate CYP metabolizer, and wherein the CYP is selected from the group consisting of: CYP1A2, CYP2B6, CYP2C8, CYP2C9, CYP2C19, CYP2D6, CYP2E1, CYP3A, and CYP3A4/3A5;

A46. Any Method 1 or 1.1-1.12, or Method 2 or 2.1-2.9, or Method 3 or 3.1-3.9, or Method A1-A45, further comprising determining that the human subject is stable on alpha-blocker therapy prior to initiating treatment with the selective CB2 receptor agonist;

A47. Any Method 1 or 1.1-1.12, or Method 2 or 2.1-2.9, or Method 3 or 3.1-3.9, or Method A1-A46, wherein the selective CB2 receptor agonist increases internalization of the CB2 receptor in a cell to at least 75%, at least 80%, at least 85%, at least 90%, at least 95%, at least 96%, at least 97%, at least 98%, or at least about 99% the level of internalization that would occur if the cell were contacted with CP55,940;

A48. Any Method 1 or 1.1-1.12, or Method 2 or 2.1-2.9, or Method 3 or 3.1-3.9, or Method A1-A47, wherein the selectivity of the selective CB2 receptor agonist is, or has previously been identified as being, at least 50-fold, at least 100-fold, at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB2 receptor relative to the human CB1 receptor;

A49. Any Method 1 or 1.1-1.12, or Method 2 or 2.1-2.9, or Method 3 or 3.1-3.9, or Method A1-A48, wherein the method further comprises:

reducing the dosage amount of the selective CB2 receptor agonist (e.g., APD371) during the administration of an agent described in paragraph a or paragraph b of Method 1.5; or

reducing the dosage amount of an agent described in paragraph a or paragraph b of Method 1.5 during the administration of the selective CB2 receptor agonist (e.g., APD371);

titrating the selective CB2 receptor agonist (e.g., APD371) in the presence of an agent described in paragraph a-c of Method 1.6; or

titrating an agent described in paragraph a or paragraph c of Method 1.6 in the presence of the selective CB2 receptor agonist (e.g., APD371);

A50. Any Method 1 or 1.1-1.12, or Method 2 or 2.1-2.9, or Method 3 or 3.1-3.9, or Method A1-A49, wherein the method further comprises co-administering a co-therapeutic compound with the selective CB2 receptor agonist; for example:
analgesic compounds such as:
   acetaminophen; and
   NSAIDs, for example aspirin, choline and magnesium salicylates, choline salicylate, celecoxib, diclofenac potassium or sodium, diflunisal, etodolac, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, salsalate, sodium salicylate, sulindac, tolmetin sodium or valdecoxib;
gastrointestinal anti-inflammatory agents, for example 5-aminosalicylates (5-ASA), mesalamine, sulfasalazine and vedolizumab;
immunosuppressive agents, for example azathioprine;
purine antagonists, for example 6-mercaptopurine;
oral corticosteroid therapeutics, for example prednisone, budesonide, or equivalent steroids;
anti-inflammatory agents, for example anti-TNF-α agents, for example infliximab, adalimumab, ustekinumab, and certolizumab pegol;
probiotics, for example Culturelle, Saccharomyces boulardii;
antibiotics used for the treatment of Crohn's Disease for example ciprofloxacin and metronidazole; and
antidiarrheals, for example loperamide and diphenoxylate with atropine.

A51. Any Method 1 or 1.1-1.12, or Method 2 or 2.1-2.9, or Method 3 or 3.1-3.9, or Method A1-A50, wherein the selective CB2 receptor agonist is APD371; the daily dose is from 150 mg to 450 mg; and the daily dose is administered in increments up to three times per day; for example in three doses per day of 50 mg; or three doses per day of 75 mg, or three doses per day of 100 mg, or three doses per day of 125 mg, or three doses per day of 150 mg.

The present disclosure further provides a method (Method 4) of selecting a candidate for treatment with a selective CB2 receptor agonist; the method comprising detecting the presence or absence of a risk factor in a human subject in need of such treatment; wherein the risk factor is as defined in Methods 1 and 1.1 et seq.; and choosing a human subject in which the risk factor is absent. In some embodiments, the method further comprises selecting a human subject having a risk factor for a modified treatment regime with a dose of the selective CB2 receptor agonist that is lower than the dose of the selective CB2 receptor agonist that is given to a subject where the risk factor is absent.

In a further aspect, the disclosure provides a method (Method 5) of treating a human subject in need of treatment with a CB₂ receptor agonist, such as a selective CB₂ receptor agonist. The method involves identifying a human subject in need of treatment with a selective CB₂ receptor agonist; determining the risk of, or presence of, a low heart rate and/or low blood pressure in the human subject; and either administering a therapeutically effective amount of the selective CB₂ receptor agonist to the human subject if the human subject does not have a risk of, or the presence of, a low heart rate and/or low blood pressure, or not administering a therapeutically effective amount of the selective CB₂ receptor agonist to the human subject if the human subject has a risk of, or the presence of, a low heart rate and/or low blood pressure.

In a further aspect, the disclosure provides a method (Method 6) of treating a human subject in need of treatment with a selective CB₂ receptor agonist comprising administering a selective CB₂ receptor agonist to the human subject. The method further involves evaluating the heart rate and/or blood pressure of the human subject after administration of the selective CB₂ receptor agonist. Administration of the selective CB₂ receptor agonist to the human subject is continued if the human subject does not have a low heart rate and/or low blood pressure, or discontinued if the human subject has a low heart rate and/or low blood pressure.

In a further aspect, the disclosure provides a method (Method 7) of treating a human subject in need of treatment with a selective CB₂ receptor agonist. The method involves determining the risk of, or presence of, a low heart rate and/or low blood pressure in the human subject and selecting a human subject that does not have the risk of, or presence of, a low heart rate and/or low blood pressure. The selected subject is administered a therapeutically effective amount of the selective CB₂ receptor agonist to the selected human subject.

In a further aspect, the disclosure provides a method (Method 8) of treating a human subject in need of treatment with a selective CB₂ receptor agonist. This method involves determining the risk of, or presence of, a low heart rate and/or low blood pressure in the human subject and selecting a human subject that has the risk of, or presence of, a low heart rate and/or low blood pressure. The selected subject is not administered a therapeutically effective amount of the selective CB₂ receptor agonist to the selected human subject.

In a further aspect, the disclosure provides a method (Method 9) of treating a human subject in need of treatment with a selective CB₂ receptor agonist. The method involves determining the risk of, or presence of, a low heart rate and/or low blood pressure in the human subject and measuring and/or evaluating the heart rate and/or blood pressure and/or a condition related thereto for the human subject. The subject is selected if the subject has a normal heart rate and/or a normal blood pressure and is administered a therapeutically effective amount of the selective CB₂ receptor agonist to the human subject.

In a further aspect, this disclosure is directed to a method (Method 10) of treating a human subject in need of treatment with a selective CB₂ receptor agonist. The method involves administering a therapeutically effective amount of the selective CB₂ receptor agonist to the human subject, wherein the human subject has previously been determined to not be at risk of, or have, a low heart rate and/or low blood pressure.

In a further aspect, the disclosure features a method (Method 11) of treating a human subject in need of treatment with a selective CB₂ receptor agonist. The method comprises administering a therapeutically effective amount of the selective CB₂ receptor agonist to the human subject, wherein the human subject has previously been determined to have a normal heart rate and/or normal blood pressure after a previous administration of the selective CB₂ receptor agonist.

In an further aspect, this disclosure provides yet a method (Method 12) of treating a human subject in need of treatment with a selective CB₂ receptor agonist. The method comprises the steps of: evaluating the heart rate and/or blood pressure of the human subject prior to administration of a selective CB₂ receptor agonist; administering a therapeutically effective amount of the selective CB₂ receptor agonist to the human subject; evaluating the heart rate and/or blood pressure of the human subject after administration of the selective CB₂ receptor agonist; determining that the heart rate and/or blood pressure after administration of the selective CB₂ receptor agonist is not decreased by a defined amount compared to the heart rate and/or blood pressure prior to administration of the selective CB₂ receptor agonist; and continuing administration of the selective CB₂ receptor agonist to the human subject.

In a further aspect, this disclosure provides a method (Method 13) of treating a human subject in need of treatment with a selective CB₂ receptor agonist. The method comprises the steps of: evaluating the heart rate and/or blood pressure of the human subject prior to administration of a selective CB₂ receptor agonist; administering a therapeutically effective amount of the selective CB₂ receptor agonist to the human subject; evaluating the heart rate and/or blood pressure of the human subject after administration of the selective CB₂ receptor agonist; determining that the heart rate and/or blood pressure of the human subject after administration of the selective CB₂ receptor agonist is a normal heart rate and/or normal blood pressure; and continuing administration of the selective CB₂ receptor agonist to the human subject.

In a further aspect, this disclosure features a method (Method 14) of treating a human subject in need of treatment with a selective CB₂ receptor agonist. The method involves administering a therapeutically effective amount of a selective CB₂ receptor agonist to the human subject, wherein the human subject has previously been determined to have a heart rate and/or blood pressure after an administration of the selective CB₂ receptor agonist that is not decreased by a defined amount compared to heart rate and/or blood pressure prior to the previous administration of the selective CB₂ receptor agonist.

In a further aspect, a method (Method 15) of treating a human subject in need of treatment with a selective CB₂ receptor agonist is provided. The method comprises administering a therapeutically effective amount of a selective CB₂ receptor agonist to the human subject, wherein the human subject has previously been determined to have a normal heart rate and/or normal blood pressure after an administration of the selective CB₂ receptor agonist.

In a further aspect, a method (Method 16) of treating a human subject in need of treatment with a selective CB₂ receptor agonist is featured. The method comprises determining if the human subject is administered an agent that reduces heart rate and/or reduces blood pressure and/or presents the risk of a reduced heart rate and/or blood pressure in the presence of a selective CB₂ receptor agonist. Administration of the agent to the human subject is continued and the selective CB₂ receptor agonist is not administered.

In a further aspect, the disclosure provides a method (Method 17) of treating a human subject in need of treatment with a selective CB₂ receptor agonist, wherein the method involves determining if the human subject is administered an agent that, in the presence of a selective CB₂ receptor agonist, reduces heart rate and/or reduces blood pressure and/or presents the risk of a reduced heart rate and/or blood pressure, and administering a therapeutically effective amount of the selective CB₂ receptor agonist to the human subject if the human subject is not currently being administered the agent.

In a further aspect, the disclosure provides method (Method 18) of treating a human subject in need of treatment with a selective CB₂ receptor agonist, wherein the method involves determining if the human subject is administered an agent that, in the presence of a selective CB₂ receptor agonist, reduces heart rate and/or reduces blood pressure and/or presents the risk of a reduced heart rate and/or blood pressure, and administering a therapeutically effective amount of the selective CB₂ receptor agonist to the human subject after the human subject has discontinued the agent.

In a further aspect, the disclosure provides a method (Method 19) of treating a human subject in need of treatment with a selective CB₂ receptor agonist. The method involves the steps of: administering a first dosage amount of a selective CB₂ receptor agonist to the human subject; evaluating the heart rate and/or blood pressure and/or a condition related thereto for the human subject; selecting the human subject if the human subject has a normal heart rate and/or blood pressure; and administering a second dosage amount of the selective CB₂ receptor agonist to the selected human subject, wherein the second dosage amount of the selective CB₂ receptor agonist is the same or greater than the first dosage amount of the selective CB₂ receptor agonist.

In a further aspect, the disclosure features a method (Method 20) of treating a human subject in need of treatment with a selective CB₂ receptor agonist. The method involves the steps of: administering a first dosage amount of a selective CB₂ receptor agonist to the human subject; determining the risk of, or presence of, a low heart rate and/or low blood pressure in the human subject; and administering a reduced dosage amount of the selective CB₂ receptor agonist to a human subject at risk of, or with, a low heart rate and/or low blood pressure, wherein the reduced dosage amount is a lower dosage than the first dosage amount.

In a further aspect, the disclosure provides a method (Method 21) of treating a human subject in need of treatment with a selective CB₂ receptor agonist, the method comprising identifying a human subject at risk of, or with, a low heart rate and/or low blood pressure, and administering a lower dose than the standard dose of a selective CB₂ receptor agonist to the human subject.

In an further aspect, the disclosure features a method (Method 21) of treating a human subject presenting with pain (e.g., acute and/or chronic inflammatory pain (such as pain associated with osteoarthritis), acute and/or chronic neuropathic pain (such as diabetic neuropathy pain), pain associated with endometriosis, abdominal pain associated with inflammatory bowel disease, chemotherapy-induced pain, pain associated with interstitial cystitis, migraine, or non-radicular low back pain) with a dose greater than or equal to 25 mg (e.g., 25, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, or 200 mg) of APD371, wherein the dose is orally administered once or twice daily for at least 12 weeks, thereby reducing the pain suffered by the human subject. In some embodiments, the dose of APD371 is 100 mg or greater. In certain embodiments, the dose of APD371 is 100 mg to 200 mg. In some embodiments, the dose of APD371 is about 50 mg or 50 mg. In some embodiments, the dose of APD371 is about 75 mg or 75 mg. In some embodiments, the dose of APD371 is about 100 mg or 100 mg. In other embodiments, the dose of APD371 is about 200 mg or 200 mg. In other embodiments, the dose of APD371 is less than 200 mg. In other embodiments, the dose of APD371 is less than 100 mg. In other embodiments, the dose of APD371 is less than 75 mg. In certain embodiments, the heart rate and/or blood pressure of the human subject is measured before and after administration. In certain embodiments, the subject is not administered APD371 if the heart rate and/or blood pressure is not normal prior to the initiation of treatment. In certain embodiments, the subject is not administered APD371 further if the heart rate and/or blood pressure is not normal after administration of APD371. In certain embodiments, the subject is not administered APD371 after initial administration of APD371 if the heart rate and/or blood pressure after administration reduces by 5% or greater, or 10% or greater, after administration of APD371 (e.g., 0.5 hours, 1 hour, 2 hours, 4 hours, 6 hours, 8 hours, 10 hours, 12 hours, 14 hours, 16 hours, 18 hours, 20 hours, 22 hours, or 24 hours after administration). In certain embodiments, a human subject having, or at risk of developing, a low heart rate or low blood pressure is not administered APD371. In certain embodiments, a human subject having, or at risk of developing, orthostatic hypotension is not administered APD371. In certain embodiments, a human subject having, or at risk of developing, bradycardia is not administered APD371. In certain embodiments, a human subject having, or at risk of developing severe renal impairment or severe hepatic impairment are administered a lower dose than the standard dose of APD371. In certain embodiments, the lower dose is 75 mg, 50 mg, 25 mg, or 12.5 mg.

In an further aspect, the disclosure features a method (Method 22) of treating a human subject presenting with fibrosis (e.g., lung fibrosis (e.g., IPF); renal fibrosis, kidney fibrosis) with a dose greater than or equal to 25 mg (e.g., 25 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, or 200 mg) of APD371, wherein the dose is orally administered once or twice daily for at least 12 weeks, thereby reducing the fibrosis or progression to fibrosis suffered by the human subject. In some embodiments, the dose of APD371 is 100 mg or greater. In certain embodiments, the dose of APD371 is 100 mg to 200 mg. In some embodiments, the dose of APD371 is about 50 mg or 50 mg. In some embodiments, the dose of APD371 is about 75 mg or 75 mg. In some embodiments, the dose of APD371 is about 100 mg or 100 mg. In other embodiments, the dose of APD371 is about 200 mg or 200 mg. In other embodiments, the dose of APD371 is less than 200 mg. In other embodiments, the dose of APD371 is less than 100 mg. In certain embodiments, the heart rate and/or blood pressure of the human subject is measured before and after administration. In certain embodiments, the subject is not administered APD371 if the heart rate and/or blood pressure is not normal prior to the initiation of treatment. In certain embodiments, the subject is not administered APD371 further if the heart rate and/or blood pressure is not normal after administration of APD371. In certain embodiments, the subject is not administered APD371 after initial administration of APD371 if the heart rate and/or blood pressure after administration reduces by 5% or greater, or 10% or greater, after administration of APD371 (e.g., 0.5 hours, 1 hour, 2 hours, 4 hours, 6 hours, 8 hours, 10 hours, 12 hours, 14 hours, 16 hours, 18 hours, 20 hours, 22 hours, or 24 hours after administration). In certain embodiments, a human subject having, or at risk of developing, a low heart rate or low blood pressure is not administered APD371. In certain embodiments, a human subject having, or at risk of developing, orthostatic hypotension is not administered APD371. In certain embodiments, a human subject having, or at risk of developing, bradycardia is not administered APD371. In certain embodiments, a human subject having, or at risk of developing severe renal impairment or severe hepatic impairment are administered a lower dose than the standard dose of APD371. In certain embodiments, the lower dose is 75 mg, 50 mg, 25 mg, or 12.5 mg.

The present disclosure further provides the use of a selective CB₂ receptor agonist in determining low blood pressure or low heart rate, in the treatment of a pain condition; and a selective CB₂ receptor agonist for use in determining low blood pressure or low heart rate in treating a pain condition. In some embodiments, the pain condition is selected from:
bone pain; joint pain; muscle pain; dental pain; migraine and other headache pain; inflammatory pain including acute inflammatory pain and chronic inflammatory pain; acute and/or chronic neuropathic pain; pain that occurs as an adverse effect of therapeutics;
pain associated with a disorder selected from: osteoarthritis, cancer, multiple sclerosis, allergic reactions, nephritic syndrome, scleroderma, thyroiditis, diabetic neuropathy, fibromyalgia, HIV related-neuropathy, neuralgias, sciatica, and autoimmune conditions;
chemotherapy-induced pain; acute post-operative pain; abdominal pain associated with inflammatory bowel disease (IBD); non-radicular low back pain; pain from liver fibrosis, primary biliary cirrhosis, nonalcoholic steatohepatitis, renal fibrosis, endometriosis, and interstitial cystitis; hyperalgesia; allodynia; inflammatory hyperalgesia; neuropathic hyperalgesia; acute nociception; osteoporosis; and multiple sclerosis-associated spasticity.

The following embodiments apply to some or all of the above Method 1, 1.1-1.9, 2, 2.1-2.5, 3, 3.1-3.6, A1-A51 and 4-22.

In some embodiments of the present methods, for example Methods 1 and 1.1-1.9, the presence or absence of a risk of a low heart rate and/or low blood pressure, is detected, wherein the risk is, inter alia, one or more of the conditions described in paragraphs a-f of Method 1.6, and in particular wherein the subject is has been previously administered one or more agents as described in paragraphs a-c of Method 1.6. In such embodiments, the prior administration can be within 6 months, within 5 months, within 3 months, within 2 months, within a month, within 3 weeks, within 2 weeks, within 1 week, within 6 days, within 5 days, within 4 days, within 3 days, within 2 days, with a day of the proposed administration of the selective CB₂ receptor agonist (e.g., APD371).

In some embodiments of each of the foregoing Methods, the selective CB₂ receptor agonist is APD371.

In one embodiment, the risk of a low heart rate and/or low blood pressure is the administration of, or the effect of the administration of, a drug other than the selective CB₂ receptor agonist. In certain instances, the drug or agent increases the exposure of the selective CB2 receptor agonist. In some instances, the drug or agent is a CYP inhibitor. In some instances, the drug or agent slows the metabolism of the selective CB₂ receptor agonist. In some instances, the presence of the drug or agent increases the accumulation of a metabolite of the selective CB₂ receptor agonist in the human subject compared to the absence of the drug or agent. In some instances, the drug or agent competes with the selective CB₂ receptor agonist for protein binding (e.g., binding to human serum albumin, lipoprotein, glycoprotein, or a globulin, βglobulin, or γ globulin). In some instances, the drug or agent is a nitrate, an alpha-blocker, an anti-hypertensive drug, or a vasodilator. In some instances, the drug or agent that slows heart rate is digoxin, amiodarone, or a beta-blocker. In certain instances, the drug or agent is alcohol. In some instances, the drug or agent causes QT prolongation.

In one embodiment, the risk of, or presence of, a low heart rate and/or low blood pressure is a medical condition. In some instances, the medical condition is known to decrease heart rate and/or blood pressure. In one embodiment, the medical condition is dehydration or hypovolemia. In one embodiment, the risk of, or presence of, a low heart rate and/or low blood pressure is a history (i.e., an occurrence within the six months prior to administration of the selective CB2 receptor agonist) of at least one of the following: dizziness, lightheadedness, hypotension, syncope, hemodynamic instability, bradycardia, aortic stenosis, myocardial infarction, ischemia, heart failure, and a conduction abnormality. In some instances, the risk of, or presence of, a low heart rate and/or low blood pressure is a preexisting condition that predisposes the human subject to reduced heart rate and/or reduced blood pressure. In certain instances, the risk of, or presence of, a low heart rate and/or low blood pressure is being 60 years of age or older, 65 years of age or older, 70 years of age or older, 75 years of age or older, 80 years of age or older, 85 years of age or older, 90 years of age or older, or 95 years of age or older. In some instances, the risk of, or presence of, a low heart rate and/or low blood pressure is being a poor CYP metabolizer. In some instances, the risk of, or presence of, a low heart rate and/or low blood pressure is being an intermediate CYP metabolizer. In some embodiments, the CYP is selected from the group consisting of: CYP1A2, CYP2B6, CYP2C8, CYP2C9, CYP2C19, CYP2D6, CYP2E1, CYP3A, and CYP3A4/3A5. In certain instances, the risk of, or presence of, a low heart rate and/or low blood pressure is a cerebrovascular disease. In some instances, the risk of, or presence of, a low heart rate and/or low blood pressure is identified by at least one of the following: dizziness, lightheadedness, fainting, headache, nausea, hypotension, syncope, shock, hemodynamic instability, bradycardia, aortic stenosis, myocardial infarction, ischemia, heart failure, and a conduction abnormality.

In one embodiment, the low heart rate is less than, or less than about, 60, 55, 50, 45, or 40 beats per minute (bpm). In certain instances, the low heart rate is less than, or less than about, 50 bpm. In some instances, the low heart rate is less than, or less than about, 50 bpm and, the low heart rate is at least, or at least about, a 10 bpm reduction from the heart rate prior to administration of the selective CB₂ receptor agonist.

In one embodiment, the low blood pressure is a systolic blood pressure of less than, or less than about, 120, 115, 110, 105, 100, 95, 90, 85, 80, 75, or 70 mmHg. In some instances, the low blood pressure is a systolic blood pressure less than, or less than about, 90 mmHg. In some instances, the low blood pressure is a diastolic blood pressure of less than, or less than about, 80, 75, 70, 65, 60, 55, or 50 mmHg. In certain instances, the low blood pressure is a diastolic blood pressure less than, or less than about, 60 mmHg. In some instances, the low blood pressure is a diastolic blood pressure less than, or less than about, 50 mmHg. In some instances, the low blood pressure is a systolic blood pressure less than, or less than about 90 mmHg, and wherein the diastolic blood pressure less than, or less than about, 50 mmHg, and wherein the low blood pressure is at least a 10 mmHg reduction from the systolic and/or diastolic blood pressure prior to administration of the selective CB₂ receptor agonist.

In one embodiment, the decrease by a defined amount is a decrease in heart rate of, of about, of at least, or of at least about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In certain instances, the decrease by a defined amount is a decrease from baseline systolic blood pressure of, of about, of at least, or of at least about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In some instances, the decrease by a defined amount is a decrease from baseline diastolic blood pressure of, of about, of at least, or of at least about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%. In certain instances, the decrease by a defined amount is a decrease in heart rate of, of about, of at least, or of at least about, 1, 2, 3, 4, 5, 6, 7, 8, 9, 10, 11, 12, 13, 14, 15, 16, 17, 18, 19, or 20 bpm. In some instances, the decrease by a defined amount is a decrease in systolic blood pressure of, of about, of at least, or of at least about, 1, 2, 3, 4, 5, 6, 7, 8, 9, 10, 15, 20, or 25 mmHg. In some instances, the decrease by a defined amount is a decrease in diastolic blood pressure of, of about, of at least, or of at least about, 1, 2, 3, 4, 5, 6, 7, 8, 9, 10, 15, 20, or 25 mmHg.

In one embodiment, the methods described above further include the step of measuring and/or evaluating the heart rate and/or blood pressure of the human subject following administration of the second dosage amount.

In one embodiment, the human subject has been identified as having a risk factor for a low heart rate and/or low blood pressure prior to administration of the first dosage amount of the selective CB₂ receptor agonist.

In another embodiment, the selective CB₂ receptor agonist is APD371, and the first dosage amount is selected from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 475 mg, and 500 mg. In some instances, the selective CB₂ receptor agonist is APD371, and wherein the first dosage amount of APD371 is, or is about, 25 mg, 50 mg, 75 mg, 100 mg, 200 mg, or 250 mg. In some instances, wherein the selective CB₂ receptor agonist is APD371, and wherein the first dosage amount of APD371 is less than, or less than about, 50 mg, 75 mg, 100 mg, 150 mg, 200 mg, or 250 mg. In one embodiment, the first dosage amount of the selective CB₂ receptor agonist is administered more than once. In certain instances, the first dosage amount is administered at a frequency of: once a day, twice a day, three times a day, or four times a day.

In another embodiment, the selective CB₂ receptor agonist is APD371, and the second dosage amount is from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg. In some instances, the selective CB₂ receptor agonist is APD371, and the second dosage amount of APD371 is, or is about, 25 mg, 50 mg, 75 mg, 100 mg, 200 mg, or 250 mg. In some instances, the selective CB₂ receptor agonist is APD371, and the second dosage amount of APD371 is greater than, or greater than about, 50 mg, 75 mg, 100 mg, 150 mg, 200 mg, or 250 mg. In certain instances, the second dosage amount is administered at a frequency selected from: once a day, twice a day, three times a day, and four times a day.

In some embodiments, the methods described above further involve measuring and/or evaluating the heart rate and/or blood pressure of the human subject following administration of the second dosage amount.

In certain embodiments, the selective CB₂ receptor agonist is APD371, and the reduced dosage amount is from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 475 mg. In certain instances, the selective CB₂ receptor agonist is APD371, and the reduced dosage amount of APD371 is, or is about, 25 mg, 50 mg, 75 mg, 100 mg, 200 mg, or 250 mg. In some instances, the selective CB₂ receptor agonist is APD371, and the reduced dosage amount of APD371 is less than, or less than about, 50 mg, 75 mg, 100 mg, 150 mg, 200 mg, or 250 mg. In certain cases, the reduced dosage amount is administered at a frequency of: once a day, twice a day, three times a day, or four times a day.

In some embodiments, the methods described above further include measuring and/or evaluating the heart rate and/or blood pressure of the human subject following administration of the reduced dosage amount.

In some embodiments, the selective CB₂ receptor agonist is APD371, and the standard dose is selected from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg. In some instances, the standard dose is administered at a frequency of: once a day, twice a day, three times a day, or four times a day.

In some embodiments, the selective CB₂ receptor agonist is APD371, and the lower dose is selected from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 475 mg. In some instances, the selective CB₂ receptor agonist is APD371, and the lower dose is less than, or less than about, 50 mg, 75 mg, 100 mg, 150 mg, 200 mg, or 250 mg. In certain instances, the lower dose is, or is about, 50 mg. In some instances, the lower dose is, or is about, 100 mg. In some instances, the lower dose is administered at a frequency of: once a day, twice a day, three times a day, or four times a day. In certain instances, the methods described herein further include a step of measuring and/or evaluating the heart rate and/or blood pressure of the human subject following administration of the lower dosage amount.

In certain embodiments, the methods described herein further include a step of monitoring the human subject for an adverse reaction following administration of the selective CB₂ receptor agonist. In some embodiments, the methods described herein further include a step of evaluating the heart rate and/or blood pressure and/or a condition related thereto for the human subject following administration of the selective CB₂ receptor agonist. In some embodiments, evaluating the heart rate and/or blood pressure of the human subject comprises measuring the heart rate and/or blood pressure of the human subject. In some instances, the heart rate and/or blood pressure of the human subject is evaluated about, or at least about, 0.5, 1, 1.5, 2, 2.5, 3, 3.5, 4, 4.5, 5, 5.5, or 6 hours following administration of the selective CB₂ receptor agonist.

In one embodiment, the human subject is determined to be at risk of, or have, a low heart rate and/or low blood pressure based on at least one determination selected from the group consisting of: (i) determining by electrocardiogram that the human subject has a low heart rate; (ii) determining by vital signs that the human subject has a low heart rate; (iii) determining by vital signs that the human subject has a low systolic and/or diastolic blood pressure; (iv) determining that the human subject has a history of low heart rate and/or low systolic and/or diastolic blood pressure and/or condition related thereto; (v) determining that the human subject has impaired elimination of the selective CB₂ receptor agonist; and (vi) determining that the human subject is a poor CYP metabolizer. In some embodiments, the human subject is determined to not be at risk of, or have, a low heart rate and/or low blood pressure based on at least one determination selected from the group consisting of: (i) determining by electrocardiogram that the human subject does not have a low heart rate; (ii) determining by vital signs that the human subject does not have a low heart rate; (iii) determining by vital signs that the human subject does not have a low systolic and/or diastolic blood pressure; (iv) determining that the human subject does not have a history of low heart rate and/or low systolic and/or diastolic blood pressure and/or condition related thereto; (v) determining that the human subject does not have impaired elimination of the selective CB₂ receptor agonist; and (vi) determining that the human subject is not a poor CYP metabolizer.

In some embodiments, the selective CB₂ receptor agonist is APD371, and the amount of APD371 is selected from, or from about, 10 mg, 15 mg, 20 mg, 25 mg, 30 mg, 35 mg, 40 mg, 45 mg, 50 mg, 60 mg, 65 mg, 70 mg, 75 mg, 80 mg, 85 mg, 90 mg, 95 mg, 100 mg, 110 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg. In certain instances, the selective CB₂ receptor agonist is APD371, wherein the amount of APD371 is, or is about, 10 mg, 25 mg, 50 mg, 75 mg, 100 mg, 150 mg, or 200 mg. In some instances, the selective CB₂ receptor agonist is administered once per day. In some instances, the selective CB₂ receptor agonist is administered twice per day. In some instances, the selective CB₂ receptor agonist is administered three times per day. In certain instances, the selective CB₂ receptor agonist is administered four times per day.

In some embodiments, the selective CB₂ receptor agonist is APD371, and the maximum dose of APD371 is, or is about, 10 mg, 25 mg, 50 mg, 75 mg, 100 mg, 125 mg, 150 mg, 175 mg, or 200 mg per administration. In some embodiments, the selective CB₂ receptor agonist is APD371, and the maximum dose of APD371 is, or is about, 75 mg, 150 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg per day. In some instances, the selective CB₂ receptor agonist is APD371, and the amount of APD371 is less than or equal to 200 mg per administration. In some instances, the selective CB₂ receptor agonist is APD371, and the amount of APD371 is less than, or less than about, 200 mg per administration.

In some embodiments, the daily dosage of the selective CB₂ receptor agonist, for example APD371, is from 75 mg to 600 mg, for example from 150 mg to 450 mg; for example from 150 mg to 300 mg. The daily dosage can be administered in a single dose, or in multiple doses, e.g., twice, three times or four times per day. For example, a daily dose of 150 mg can be administered in a day as three doses of 50 mg each, and a daily dose of 300 mg can be administered in a day as three doses of 100 mg each.

In certain embodiments, the normal heart rate is at least, or at least about, 60 bpm. In some embodiments, the normal heart rate is about 60 bpm to about 100 bpm. In certain instances the heart rate is one of: a resting heart rate, a supine heart rate, or a standing heart rate.

In some embodiments, the normal blood pressure is a systolic blood pressure of about 90 mmHg to about 119 mmHg. In some embodiments, the normal blood pressure is a diastolic blood pressure of about 60 mmHg to about 79 mmHg. In certain instances, the blood pressure is selected from systolic blood pressure and diastolic blood pressure. In some instances, the systolic blood pressure is selected from: a resting systolic blood pressure, a supine systolic blood pressure, and a standing systolic blood pressure. In certain instances, the diastolic blood pressure is selected from: a resting diastolic blood pressure, a supine diastolic blood pressure, and a standing diastolic blood pressure.

In some embodiments, the human subject is not part of a multi-center, placebo-controlled, double-blind trial designed to: (i) examine the safety or efficacy of the selective CB₂ receptor agonist, and/or (ii) have data therefrom submitted to a regulatory agency for approval of the selective CB₂ receptor agonist for treatment of human subjects.

In certain embodiments, the human subject is elderly.

In certain embodiments, the hypotension is postural hypotension, orthostatic hypotension, or postprandial hypotension.

In some embodiments, the selective CB₂ receptor agonist is administered orally. In certain instances, the selective CB₂ receptor agonist is in the form of a tablet or capsule. In some instances, the selective CB₂ receptor agonist is in an immediate-release dosage form.

In certain embodiments, a condition related to low heart rate and/or low blood pressure is selected from at least one of the following: dizziness, lightheadedness, fainting, headache, nausea, hypotension, syncope, shock, hemodynamic instability, bradycardia, aortic stenosis, myocardial infarction, ischemia, heart failure, and a conduction abnormality.

In one embodiment, the treatment is the treatment or prevention of a CB₂ receptor-mediated disorder. In some instances, the treatment is the treatment or prevention of a CB₂ receptor-mediated disorder selected from: pain associated with osteoarthritis, chemotherapy-induced pain, neuropathic pain, acute post-operative pain, abdominal pain associated with inflammatory bowel disease (IBD), non-radicular low back pain, liver fibrosis, primary biliary cirrhosis, nonalcoholic steatohepatitis, renal fibrosis, osteoarthritis, endometriosis, interstitial cystitis, and migraine. In certain instances, the treatment is the treatment of acute and/or chronic inflammatory pain. In some instances, the treatment is the treatment of acute and/or chronic neuropathic pain.

In some embodiments, the human subject has a poor metabolizer or intermediate metabolizer phenotype for a CYP. In some embodiments, the CYP is selected from: CYP1A2, CYP2B6, CYP2C8, CYP2C9, CYP2C19, CYP2D6, CYP2E1, CYP3A, CYP3A4/3A5, and/or another relevant CYP known to one of skill in the art. In some embodiments, the human subject has been genotyped for a CYP reaction. In some embodiments, the human subject has been phenotyped for a CYP reaction.

In some embodiments, the methods described above further involve determining that the human subject is stable on alpha-blocker therapy prior to initiating treatment with the selective CB₂ receptor agonist.

In some embodiments, the selective CB₂ receptor agonist increases internalization of the CB₂ receptor in a cell to at least 75%, at least 80%, at least 85%, at least 90%, at least 95%, at least 96%, at least 97%, at least 98%, or at least about 99% the level of internalization that would occur if the cell were contacted with CP55,940. In some embodiments, the CB₂ receptor agonist that increases internalization of the CB₂ receptor to at least 90% of CP55,940 is selected from: Compound B, Compound E, Compound G, and Compound H. CP55,940 has the following chemical structure:

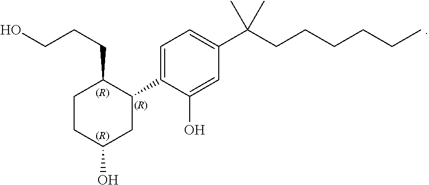

In certain embodiments, the selectivity of the selective CB₂ receptor agonist is at least 50-fold, at least 100-fold, at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB₂ receptor relative to the human CB₁ receptor.

In some embodiments, the selectivity of the selective CB₂ receptor agonist has previously been identified as being at least 50-fold, at least 100-fold, at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB₂ receptor relative to the human CB₁ receptor.

In some embodiments, the selective CB₂ receptor agonist is APD371, the therapeutically effective amount of APD371 is about 25-100 mg per administration, and the APD371 is administered twice daily.

In some embodiments, the selective CB₂ receptor agonist is APD371, the therapeutically effective amount of APD371 is about 25-100 mg per administration, and the APD371 is administered three times per day.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 shows the mean change in heart rate from baseline on Day 1 in the single ascending dose study described in Example 1 (placebo and 10 mg), as measured by continuous telemetry.

FIG. 2 shows the mean change in heart rate from baseline on Day 1 in the single ascending dose study described in Example 1 (20 mg and 30 mg), as measured by continuous telemetry.

FIG. 3 shows the mean change in heart rate from baseline on Day 1 in the single ascending dose study described in Example 1 (60 mg and 120 mg), as measured by continuous telemetry.

FIG. 4 shows the mean change in heart rate from baseline on Day 1 in the single ascending dose study described in Example 1 (250 mg and 400 mg), as measured by continuous telemetry.

FIG. 5 shows the mean change in heart rate from baseline to Day 7 in the single ascending dose study described in Example 1 (placebo and 10 mg), as measured by vital signs.

FIG. 6 shows the mean change in heart rate from baseline to Day 7 in the single ascending dose study described in Example 1 (20 mg and 30 mg), as measured by vital signs.

FIG. 7 shows the mean change in heart rate from baseline to Day 7 in the single ascending dose study described in Example 1 (60 mg and 120 mg), as measured by vital signs.

FIG. 8 shows the mean change in heart rate from baseline to Day 7 in the single ascending dose study described in Example 1 (250 mg and 400 mg), as measured by vital signs.

FIG. 9 shows the mean change in systolic blood pressure from baseline to Day 7 in the single ascending dose study described in Example 1 (placebo and 10 mg), as measured by vital signs.

FIG. 10 shows the mean change in systolic blood pressure from baseline to Day 7 in the single ascending dose study described in Example 1 (20 mg and 30 mg), as measured by vital signs.

FIG. 11 shows the mean change in systolic blood pressure from baseline to Day 7 in the single ascending dose study described in Example 1 (60 mg and 120 mg), as measured by vital signs.

FIG. 12 shows the mean change in systolic blood pressure from baseline to Day 7 in the single ascending dose study described in Example 1 (250 mg and 400 mg), as measured by vital signs.

FIG. 13 shows the mean change in diastolic blood pressure from baseline to Day 7 in the single ascending dose study described in Example 1 (placebo and 10 mg), as measured by vital signs.

FIG. 14 shows the mean change in diastolic blood pressure from baseline to Day 7 in the single ascending dose study described in Example 1 (20 mg and 30 mg), as measured by vital signs.

FIG. 15 shows the mean change in diastolic blood pressure from baseline to Day 7 in the single ascending dose study described in Example 1 (60 mg and 120 mg), as measured by vital signs.

FIG. 16 shows the mean change in diastolic blood pressure from baseline to Day 7 in the single ascending dose study described in Example 1 (250 mg and 400 mg), as measured by vital signs.

FIG. 17 shows the mean change in heart rate from baseline on Day 1 in the multiple ascending dose study described in Example 2, as measured by vital signs.

FIG. 18 shows the mean change in heart rate from baseline to Day 10 in the multiple ascending dose study described in Example 2, as measured by vital signs.

FIG. 19 shows the mean change in heart rate from baseline to follow-up in the multiple ascending dose study described in Example 2, as measured by electrocardiogram (ECG).

FIG. 20 shows the mean change in heart rate from baseline on Day 1 in the multiple ascending dose study described in Example 2, as measured by telemetry.

FIG. 21 shows the mean change in heart rate from baseline to Day 10 in the multiple ascending dose study described in Example 2, as measured by vital signs.

FIG. 22 shows the mean change in systolic blood pressure from baseline on Day 1 in the multiple ascending dose study described in Example 2, as measured by vital signs.

FIG. 23 shows the mean change in systolic blood pressure from baseline to Day 10 in the multiple ascending dose study described in Example 2, as measured by vital signs.

FIG. 24 shows the mean change in diastolic blood pressure from baseline on Day 1 in the multiple ascending dose study described in Example 2, as measured by vital signs.

FIG. 25 shows the mean change in diastolic blood pressure from baseline to Day 10 in the multiple ascending dose study described in Example 2, as measured by vital signs.

The following abbreviations are used in the Figures: D=day, hr=hour, hrs=hours, PD=post dose.

### DETAILED DESCRIPTION

This disclosure provides methods of treating a human subject in need of treatment with a selective CB₂ receptor agonist taking into account the unexpected finding that CB₂ receptor agonists can reduce the heart rate and/or blood pressure of a human subject.

### Selective CB₂ Receptor Agonists

Compounds that interact with and stimulate the CB₂ receptor (which may also referred to herein as “CB₂ receptor agonists” or “CB₂ agonists”) have utility for the treatment of CB₂ receptor-mediated disorders. In certain embodiments, the agonist compound is selective for the CB₂ receptor relative to the CB₁ receptor. In some embodiments, the agonist compound is selective for the human CB₂ receptor relative to the human CB₁ receptor.

Non-limiting examples of CB₂ receptor agonist compounds are disclosed in PCT patent publications WO2011/025541, WO2012/116276, WO2012/116278, WO2012/116277, and WO2012/116279, and U.S. provisional patent application 62/084,165 (WO2016/085941), which are each incorporated herein by reference in their entirety. For example, CB₂ receptor agonist compounds include Compounds 493, 696, 699, 700, 704, 765, 820, 841, and 919 disclosed in WO2011/025541. These compounds can be prepared as disclosed in WO2011/025541.

Other CB₂ receptor agonist compounds can be identified e.g., using the Homogeneous Time-Resolved Fluorescence (HTRF®) Assay for direct cAMP measurement (Gabriel et al., ASSAY and Drug Development Technologies, 1:291-303, 2003) (see, e.g., Example 2 of WO2011/025541). Compounds can be screened for agonism of the CB₂ receptor (e.g., human CB₂ receptor) using the HTRF® assay for direct cAMP measurement (assay kit sold by Cisbio-US, Inc., Bedford, Mass.; Catalog #62AM4PEC) in recombinant CHO-K1 cells stably transfected with the CB₂ receptor. The HTRF® assay supported by the kit is a competitive immunoassay between endogenous cAMP produced by the CHO-K1 cells and tracer cAMP labeled with the dye d2. The tracer binding is visualized by a monoclonal anti-cAMP antibody labeled with Cryptate. The specific signal (i.e., fluorescence resonance energy transfer, FRET) is inversely proportional to the concentration of unlabeled cAMP in the standard or sample. An agonist of the CB₂ receptor is detected in the HTRF® assay for direct cAMP measurement when a compound decreases cAMP concentration. The HTRF® assay can also be employed to determine EC₅₀ values for CB₂ receptor agonists.

CB₂ receptor agonist compounds can also be identified using the DiscoveRx PathHunter β-arrestin assay which measures the β-arrestin binding to the CB₂ receptor upon its activation (see, e.g., Example 3 of WO2011/025541). The PathHunter-arrestin assay measures the interaction of β-arrestin with activated GPCRs using Enzyme Fragment Complementation (Yan et al., J. Biomol. Screen. 7: 451-459, 2002). A small, 42 amino acid β-galactosidase fragment, Prolink, is fused to the c-terminus of a GPCR, and β-arrestin is fused to the larger β-galactosidase fragment, EA (Enzyme Acceptor). Binding of β-arrestin to the activated GPCR causes the complementation of the two enzyme fragments, forming an active β-galactosidase enzyme which can be measured using the chemiluminescent PathHunter Flash Detection Kit (DiscoveRx, Fremont, Calif.: catalog #93-0001).

CB₂ receptor agonist compounds can also be identified using radioligand binding assays as described in Example 3 of WO2011/025541.

In certain instances, the CB₂ receptor agonist compounds identified using the HTRF® assay, the PathHunter-arrestin assay, and the radioligand binding assay can be tested for CB₂ receptor internalization to select those agonists that induce robust internalization of the receptor in order to maintain a sufficient level of signaling for sustained in vivo efficacy. In certain embodiments, such agonists are preferred in the methods described herein.

Receptor internalization can be measured using a number of methods, including but not limited to measuring a loss of labeled receptor from the cell surface (e.g., using flow cytometry) measuring the appearance of receptors internalized in the cell (e.g., in characteristic punctate intracellular vesicles), and/or measuring the return of receptors recycled to the cell surface. For example, the number, density, and/or staining intensity of granules in the cell can be quantified. Receptor internalization can be measured using any appropriate method known to those of skill in the art. For example, receptor internalization can be measured as the loss of receptors from the cell surface; as the appearance of receptors inside the cell; as the appearance of internalized receptors in intracellular vesicles; using epitope-tagged receptors; using antibody-labeled receptors; using fluorescently labeled receptors; as a change in fluorescence intensity at the cell surface and/or inside the cell; by quantifying the number, density, and/or staining intensity of fluorescent granules in the cell; measured using an immunoassay (e.g., a Western blot, immunofluorescence); using fluorescence microscopy; using a flow cytometry assay; using enzyme complementation; or using high content analysis.

The in vivo efficacy of the CB₂ receptor agonist compound can be measured for a disorder described herein. In some embodiments, in vivo efficacy is measured for pain. In some embodiments, in vivo efficacy is measured for fibrosis. In some embodiments, in vivo efficacy is measured using diagnostic criteria described herein. In some embodiments, in vivo efficacy is measured about, or at least about, 0.5, 1, 1.5, 2, 2.5, 3, 3.5, 4, 4.5, 5, 5.5, 6, 6.5, 7, 7.5, or 8 hours following dosing with a compound described herein. In some embodiments, in vivo efficacy is measured in an animal model. In some embodiments, in vivo efficacy is measured in a non-human mammal. In some embodiments, in vivo efficacy is measured in a human. In some embodiments, in vivo efficacy is measured in an animal model. In some embodiments, the animal model is a model for a CB₂ receptor-mediated disorder. In some embodiments, the animal model is a model for pain or conditions related thereto. In some embodiments, the animal model is a model for fibrosis or conditions related thereto. In some embodiments, the animal model is a Freund's complete adjuvant (FCA)-induced hyperalgesia model. In some embodiments, the animal model is a capsaicin-induced model of hyperalgesia and/or allodynia. In some embodiments, the animal model is a Zucker diabetic fatty (ZDF) rat. In some embodiments, the animal model is a streptozotocin (STZ)-treated rat. In some embodiments, the animal model is a model of neuropathic pain, such as a chronic constriction injury model of neuropathic pain. In some embodiments, the animal model is a bile duct ligation model. In some embodiments, the animal model is a hepatic fibrosis model. In some embodiments, the animal model is a NASH model. In some embodiments, the animal model is a pulmonary fibrosis model, such as a bleomycin-induced pulmonary fibrosis model. In some embodiments, the animal model is a dermal fibrosis model. In some embodiments, the animal model is a model of acute injury, such as acute kidney injury. In some embodiments, the animal model is a cholestatic liver injury model. In some embodiments, the animal model is an experimental autoimmune encephalomyelitis (EAE) model. In some embodiments, the animal model is an occlusion model of stroke. In some embodiments, the animal model is a model of atherosclerosis. In some embodiments, the animal model is a cyclophosphamide-induced cystitis model.

In one embodiment, the CB₂ receptor agonist is the compound (1aR,5aR)-2-(2,4-Difluoro-phenyl)-1a,2,5,5a-tetrahydro-1H-2,3-diaza-cyclopropa[a]pentalene-4-carboxylic Acid (2-Hydroxy-1,1-dimethyl-ethyl)-amide (“Compound A”) with the chemical structure shown below:

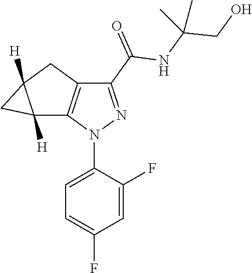 [SMILES: CC(C)(CO)NC(=O)c1nn(-c2ccc(F)cc2F)c2c1C[C@H]1C[C@@H]21]

This compound can be prepared as described in Example 1.10 of WO2011/025541 and is referred to as Compound 493 in this PCT publication.

In another embodiment, the CB₂ receptor agonist is the compound (1aS,5aS)-2-(4-Oxy-pyrazin-2-yl)-1a,2,5,5a-tetrahydro-1H-2,3-diaza-cyclopropa[a]pentalene-4-carboxylic Acid ((S)-1-Hydroxymethyl-2,2-dimethyl-propyl)-amide (“Compound B”) with the chemical structure shown below:

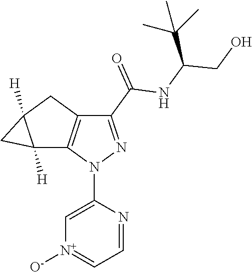 [SMILES: CC(C)(C)[C@@H](CO)NC(=O)c1nn(-c2c[n+]([O-])ccn2)c2c1C[C@@H]1C[C@H]21]

This compound can be prepared as described in Example 1.80 of WO2011/025541 and is referred to as Compound 699 in this PCT publication. This compound is also referred to herein as APD371.

As used herein “APD371” refers to (1aS,5aS)-2-(4-Oxy-pyrazin-2-yl)-1a,2,5,5a-tetrahydro-1H-2,3-diaza-cyclopropa[a]pentalene-4-carboxylic Acid ((S)-1-Hydroxymethyl-2,2-dimethyl-propyl)-amide, having the chemical structure shown above (i.e., Compound B), and all chemical and physical forms thereof, including but not limited to, APD371, amorphous forms of APD371, crystalline forms of APD371, crystalline polymorphs of APD371, crystalline habits of APD371, solvates of APD371, amorphous forms of solvates of APD371, crystalline forms of solvates of APD371, crystalline polymorphs of solvates of APD371, crystalline habits of solvates of APD371, hydrates of APD371, amorphous forms of hydrates of APD371, crystalline forms of hydrates of APD371, crystalline polymorphs of hydrates of APD371, crystalline habits of hydrates of APD371, pharmaceutically acceptable salts of APD371, amorphous forms of pharmaceutically acceptable salts of APD371, crystalline forms of pharmaceutically acceptable salts of APD371, crystalline polymorphs of pharmaceutically acceptable salts of APD371, crystalline habits of pharmaceutically acceptable salts of APD371, solvates of pharmaceutically acceptable salts of APD371, amorphous forms of solvates of pharmaceutically acceptable salts of APD371, crystalline forms of solvates of pharmaceutically acceptable salts of APD371, crystalline polymorphs of solvates of pharmaceutically acceptable salts of APD371, crystalline habits of solvates of pharmaceutically acceptable salts of APD371, hydrates of pharmaceutically acceptable salts of APD371, amorphous forms of hydrates of pharmaceutically acceptable salts of APD371, crystalline forms of hydrates of pharmaceutically acceptable salts of APD371, crystalline polymorphs of hydrates of pharmaceutically acceptable salts of APD371, crystalline habits of hydrates of pharmaceutically acceptable salts of APD371, and isotopic enrichment (e.g., deuterium) analogues of any of the above.

APD371 has demonstrated sustained efficacy in models of osteoarthritis pain, paclitaxel-induced neuropathic pain, and painful peripheral diabetic neuropathy. This compound also demonstrates >1,000-fold selectivity for the human CB₂ receptor versus the human CB₁ receptor.

This compound also demonstrated a high receptor internalization efficacy for rat and human CB₂ receptors relative to CP55,940 (105% and 96%, respectively). In an osteoarthritis pain model, this compound maintained in vivo efficacy for four hours following dosing, despite rapidly declining plasma concentrations.

In yet another embodiment, the CB₂ receptor agonist is the compound (1aS,5aS)-2-(4-Oxy-pyrazin-2-yl)-1a,2,5,5a-tetrahydro-1H-2,3-diaza-cyclopropa[a]pentalene-4-carboxylic Acid (1-Pyridin-2-yl-cyclobutyl)-amide (“Compound C”) with the chemical structure shown below:

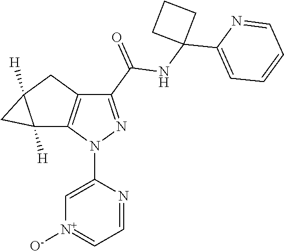 [SMILES: O=C(NC1(c2ccccn2)CCC1)c1nn(-c2c[n+]([O-])ccn2)c2c1C[C@@H]1C[C@H]21]

This compound can be prepared as described in Example 1.81 of WO2011/025541 and is referred to as Compound 700 in this PCT publication.

In a further embodiment, the CB₂ receptor agonist is the compound (1aR,5aR)-2-(4-Oxy-pyrazin-2-yl)-1a,2,5,5a-tetrahydro-1H-2,3-diaza-cyclopropa[a]pentalene-4-carboxylic Acid ((S)-2,2-Dimethyl-1-methylcarbamoyl-propyl)-amide (“Compound D”) with the chemical structure shown below:

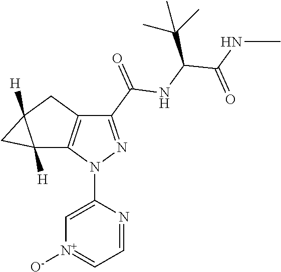 [SMILES: CNC(=O)[C@@H](NC(=O)c1nn(-c2c[n+]([O-])ccn2)c2c1C[C@H]1C[C@@H]21)C(C)(C)C]

This compound can be prepared as described in Example 1.155 of WO2011/025541 and is referred to as Compound 704 in this PCT publication.

In another embodiment, the CB₂ receptor agonist is the compound (1aS,5aS)-2-(4-Oxy-pyrazin-2-yl)-1a,2,5,5a-tetrahydro-1H-2,3-diaza-cyclopropa[a]pentalene-4-carboxylic acid (1-trifluoromethyl-cyclobutyl)-amide (“Compound E”) with the chemical structure shown below:

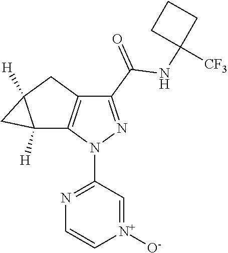 [SMILES: O=C(NC1(C(F)(F)F)CCC1)c1nn(-c2c[n+]([O-])ccn2)c2c1C[C@@H]1C[C@H]21]

This compound can be prepared as described in Example 1.118 of WO2011/025541 and is referred to as Compound 765 in this PCT publication.

In yet another embodiment, the CB₂ receptor agonist is the compound (1aR,5aR)-2-(4-Cyano-pyridin-2-yl)-1a,2,5,5a-tetrahydro-1H-2,3-diaza-cyclopropa[a]pentalene-4-carboxylic Acid (2-Hydroxy-1-hydroxymethyl-1-methyl-ethyl)-amide (“Compound F”) with the chemical structure shown below:

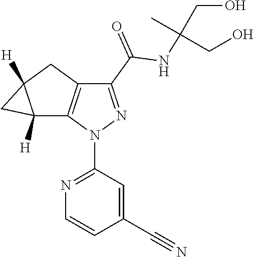 [SMILES: CC(CO)(CO)NC(=O)c1nn(-c2cc(C#N)ccn2)c2c1C[C@H]1C[C@@H]21]

This compound can be prepared as described in Example 1.125 of WO2011/025541 and is referred to as Compound 820 in this PCT publication.

In another embodiment, the CB₂ receptor agonist is the compound (1aR,5aR)-2-(4-Oxy-pyrazin-2-yl)-1a,2,5,5a-tetrahydro-1H-2,3-diaza-cyclopropa[a]pentalene-4-carboxylic acid [(S)-2-hydroxy-1-(tetrahydro-pyran-4-yl)-ethyl]-amide (“Compound G”) with the chemical structure shown below:

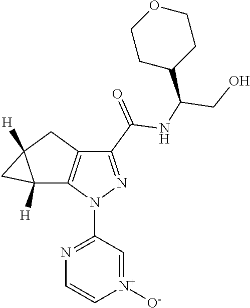 [SMILES: O=C(N[C@H](CO)C1CCOCC1)c1nn(-c2c[n+]([O-])ccn2)c2c1C[C@H]1C[C@@H]21]

This compound can be prepared as described in Example 1.116 of WO2011/025541 and is referred to as Compound 841 in this PCT publication.

In yet another embodiment, the CB₂ receptor agonist is the compound (1aS,5aS)-2-(4-Oxy-pyrazin-2-yl)-1a,2,5,5a-tetrahydro-1H-2,3-diaza-cyclopropa[a]pentalene-4-carboxylic acid (2,2,2-trifluoro-1,1-dimethyl-ethyl)-amide (“Compound H”) with the chemical structure shown below:

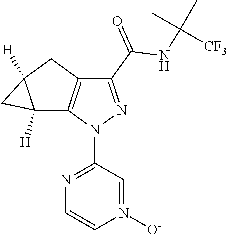 [SMILES: CC(C)(NC(=O)c1nn(-c2c[n+]([O-])ccn2)c2c1C[C@@H]1C[C@H]21)C(F)(F)F]

This compound can be prepared as described in Example 1.113 of WO2011/025541 and is referred to as Compound 919 in this PCT publication.

In some embodiments, any one of Compounds A through H described above are used in the methods described herein. In certain embodiments, any one of Compounds B, E, G, or H are used in the methods described herein. In a specific embodiment, Compound B is used in the methods described herein. In a specific embodiment, Compound H is used in the methods described herein.

The methods described below can use the compounds described above as well as pharmaceutically acceptable salts, solvates, and hydrates thereof.

The compounds described above can also be formulated with a pharmaceutically acceptable carrier as a pharmaceutical composition for use in the methods described below.

### Selectivity of an Agonist Compound for the CB₂ Receptor

In some embodiments, an agonist compound that is used in the methods described herein has selectivity for the CB₂ Receptor. In some embodiments, selectivity refers to the relative in vitro potency of a compound for the CB₂ receptor and another receptor. For example, in some embodiments, selectivity refers to the relative in vitro potency of a compound for the CB₂ receptor versus the CB₁ receptor. In some embodiments, in vitro potency is measured using a second messenger assay. In some embodiments, in vitro potency is measured using a cAMP assay. In some embodiments, selectivity is determined by comparing data generated using a β-arrestin assay. In some embodiments, selectivity is determined by comparing data generated from a GTP-yS binding assay. In some embodiments, selectivity is determined by comparing data generated from a reporter gene assay. In some embodiments, selectivity is determined by comparing data generated for a biomarker. In some embodiments, in vitro potency is quantified as EC50. In some embodiments, selectivity refers to the relative binding affinity of an agonist for the CB₂ receptor and another receptor. In some embodiments, binding affinity is quantified as Ki.

In some embodiments, selectivity is assessed for the mouse, rat, or human CB₂ receptor. In some embodiments, selectivity is assessed for the human CB₂ receptor. In some embodiments, selectivity is assessed for the CB₂ receptor versus the CB₁ receptor. In some embodiments, selectivity is assessed for the human CB₂ receptor versus the human CB₁ receptor. In some embodiments, a compound described herein exhibits about, or at least about, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1750-fold, 2000-fold, 2500-fold, 3000-fold, 3500-fold, 4000-fold, 4500-fold, 5000-fold, 6000-fold, 7000-fold, 8000-fold, 9000-fold, or 10000-fold selectivity for the CB₂ receptor versus the CB₁ receptor. In some embodiments, a compound described herein exhibits about, or at least about, 50-fold, 75-fold, 100-fold, 125-fold, 150-fold, 175-fold, 200-fold, 225-fold, 250-fold, 275-fold, 300-fold, 325-fold, 350-fold, 375-fold, 400-fold, 425-fold, 450-fold, 475-fold, 500-fold, 550-fold, 600-fold, 650-fold, 700-fold, 750-fold, 800-fold, 850-fold, 900-fold, 950-fold, 1000-fold, 1100-fold, 1200-fold, 1300-fold, 1400-fold, 1500-fold, 1750-fold, 2000-fold, 2500-fold, 3000-fold, 3500-fold, 4000-fold, 4500-fold, 5000-fold, 6000-fold, 7000-fold, 8000-fold, 9000-fold, or 10000-fold selectivity for the human CB₂ receptor versus the human CB₁ receptor.

In one embodiment, APD371, which demonstrates >1,000-fold selectivity for the human CB₂ receptor versus the human CB₁ receptor, is used in the methods described herein. In some embodiments, APD371 is an amorphous form of APD371. In some embodiments, APD371 is a crystalline form of APD371. In some embodiments, APD371 is a crystalline polymorph of APD371. In some embodiments, APD371 is a crystalline habit of APD371.

### Compositions and Formulations

The compounds described herein can be administrated in a wide variety of oral and parenteral dosage forms. One of skill in the art will understand that the dosage forms may comprise, as the active component, either a compound described herein or a pharmaceutically acceptable salt, hydrate, or solvate of a compound described herein.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound described herein in a suitable liquid vehicle and filter sterilizing the solution before filling and sealing an appropriate vial or ampule. These are just a few examples of the many appropriate methods well known in the art for preparing dosage forms.

A compound described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20th Edition, 2000, Lippincott Williams & Wilkins, (editors: Gennaro et al.).

While it is possible that a compound described herein may be administered as a raw or pure chemical, it is preferable to present the compound or active ingredient as a pharmaceutical formulation or as a composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation, or transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive, and a removable protective layer with a release liner. One of skill in the art will understand and appreciate the techniques appropriate for manufacturing a desired efficacious transdermal patch based upon the needs of one of skill in the art.

The compounds described herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds described herein or a solvate, hydrate or physiologically functional derivative thereof can be used as active ingredients in pharmaceutical compositions, specifically as CB₂ receptor agonists. The term “active ingredient,” defined in the context of a “pharmaceutical composition,” refers to a component of a pharmaceutical composition that provides the primary pharmacological effect, as opposed to an “inactive ingredient” which would generally be recognized as providing no pharmaceutical benefit.

For preparing pharmaceutical compositions from the compounds described herein, the selection of a suitable pharmaceutically acceptable carrier can be either solid, liquid, or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, one of skill in the art would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethyl cellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams, or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds described herein may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethyl cellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds described herein may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the individual administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds described herein or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds described herein as an aerosol can be prepared by processes well known to the person skilled in the art. For their preparation, for example, solutions or dispersions of the compounds described herein in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations can be prepared in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like. Certain pharmaceutically acceptable salts are listed in Berge, et al., J Pharmaceutical Sciences, 66: 1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds described herein may form solvates with standard low molecular weight solvents using methods known to one of skill in the art.

Compounds described herein can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds described herein containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems vol. 14 of the A.C.S. Symposium Series; and in Bioreversible Carriers in Drug Design, ed. Edward B. Roche, American Pharmaceutical Association and Pergamon Press, 1987.

Some embodiments include a method of producing a pharmaceutical composition for combination therapy comprising admixing at least one compound according to any of the compound embodiments disclosed herein, together with at least one known pharmaceutical agent as described herein and a pharmaceutically acceptable carrier.

It will be apparent to those skilled in the art that the dosage forms described herein may comprise, as the active component, either a compound described herein, a pharmaceutically acceptable salt of a compound described herein, a solvate or hydrate of a compound described herein, or a solvate or hydrate of a pharmaceutically acceptable salt of a compound described herein. Moreover, various hydrates and solvates of the compounds described herein and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, vol. 95, Marcel Dekker, Inc., New York, 1999. Accordingly, one aspect of the present disclosure pertains to methods of administering hydrates and solvates of compounds described herein and/or their pharmaceutical acceptable salts, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

The present disclosure includes all isotopes of atoms occurring in salts and crystalline forms thereof. Isotopes include those atoms having the same atomic number but different mass numbers. One aspect of the present disclosure includes every combination of one or more atoms in the present salts and crystalline forms thereof that is replaced with an atom having the same atomic number but a different mass number. One such example is the replacement of an atom that is the most naturally abundant isotope, such as ¹H or ¹²C, found in one the present salts and crystalline forms thereof, with a different atom that is not the most naturally abundant isotope, such as ²H or ³H (replacing ¹H), or ¹¹C, ¹³C, or ¹⁴C (replacing ¹²C). A salt wherein such a replacement has taken place is commonly referred to as being isotopically-labeled. Isotopic-labeling of the present salts and crystalline forms thereof can be accomplished using any one of a variety of different synthetic methods known to those of skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸C. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present disclosure includes compositions, such as those prepared during synthesis, preformulation, and the like, and pharmaceutical compositions, such as those prepared with the intent of using in a mammal for the treatment of one or more of the disorders described herein, comprising one or more of the present salts and crystalline forms thereof, wherein the naturally occurring distribution of the isotopes in the composition is perturbed. Another aspect of the present disclosure includes compositions and pharmaceutical compositions comprising salts and crystalline forms thereof as described herein wherein the salt is enriched at one or more positions with an isotope other than the most naturally abundant isotope. Methods are readily available to measure such isotope perturbations or enrichments, such as mass spectrometry, and for isotopes that are radio-isotopes additional methods are available, such as radio-detectors used in connection with HPLC or GC.

### Indications

The compounds described herein (e.g., APD371) are useful in the treatment or prevention of a CB₂ receptor-mediated disorder and/or the amelioration of symptoms thereof.

One aspect of the present disclosure relates to compounds or pharmaceutical compositions as described herein, for use in a method of treatment of the human or animal body by therapy.

Another aspect of the present disclosure relates to the use of compounds or pharmaceutical compositions described herein in the treatment or prevention of a CB₂ receptor-mediated disorder. In some embodiments, the CB₂ receptor-mediated disorder is one or more of the disorders described herein.

Another aspect of the present disclosure relates to the use of compositions or pharmaceutical compositions described herein in the manufacture of a medicament for treating or preventing a CB₂ receptor-mediated disorder. In some embodiments, the CB₂ receptor-mediated disorder is one or more of the disorders described herein.

Another aspect of the present disclosure relates to methods for the treatment or prevention of a CB₂ receptor-mediated disorder in an individual, comprising administering to the individual in need thereof a therapeutically effective amount of a compound or pharmaceutical composition as described herein in accordance with the methods as described herein, including Methods 1, 1.1-1.12, 2, 2.1-2.9, 3, 3.1-3.9, A1-A51 and 4-22. In some embodiments, the CB₂ receptor-mediated disorder is one or more of the disorders described herein.

Non-limiting examples of CB₂ receptor-mediated disorders are described below.

### I. Pain

In some embodiments, the CB₂ receptor-mediated disorder is pain or a condition related thereto. The CB₂ receptor plays a role in mediating the analgesic effects of cannabinoids (reviewed in Br. J. Pharmacol. 153:319-334, 2008). For example, systemic delivery of the CB₂-selective agonist AM1241 suppresses hyperalgesia induced in the carrageenan, capsaicin, and formalin models of inflammatory pain in rodents (reviewed in Br. J. Pharmacol. 153:319-334, 2008). Local (subcutaneous) or systemic administration of AM1241 also reverses tactile and thermal hypersensitivity in rats following ligation of spinal nerves in the chronic constriction injury model of neuropathic pain (Pain 93:239-245, 2001; PNAS 100(18):10529-10533, 2003), an effect which is inhibited by treatment with the CB₂-selective antagonist AM630 (PNAS 102(8):3093-8, 2005). The CB₂-selective agonist GW405833 administered systemically significantly reverses hypersensitivity to mechanical stimuli in rats following ligation of spinal nerves (Pain 143.206-212, 2009). Thus, CB₂ receptor agonists have also been shown to attenuate pain in experimental models of acute, inflammatory, and neuropathic pain, and hyperalgesia.

Accordingly, CB₂ agonists find use in the treatment and/or prophylaxis of acute nociception and inflammatory hyperalgesia, as well as the allodynia and hyperalgesia produced by neuropathic pain. For example, the agonists disclosed in the Methods herein are useful as an analgesic to treat pain arising from bone pain; joint pain; muscle pain; dental pain; migraine and other headache pain; inflammatory pain including acute inflammatory pain and chronic inflammatory pain; neuropathic pain; pain that occurs as an adverse effect of therapeutics; pain associated with a disorder selected from: osteoarthritis, cancer, multiple sclerosis, allergic reactions, nephritic syndrome, scleroderma, thyroiditis, diabetic neuropathy, fibromyalgia, HIV related-neuropathy, neuralgias, sciatica, and autoimmune conditions; acute and/or chronic inflammatory pain; acute and/or chronic neuropathic pain; chemotherapy-induced pain; acute post-operative pain; abdominal pain associated with inflammatory bowel disease (IBD); non-radicular low back pain; pain from liver fibrosis, primary biliary cirrhosis, nonalcoholic steatohepatitis, renal fibrosis, endometriosis, and interstitial cystitis; hyperalgesia; allodynia; inflammatory hyperalgesia; neuropathic hyperalgesia; acute nociception; osteoporosis; multiple sclerosis-associated spasticity; autoimmune disorders, for example an autoimmune disorder selected from the group consisting of: multiple sclerosis. Guillan-Barré syndrome, polyradiculoneuropathy, chronic inflammatory demyelination, rheumatoid arthritis, psoriatic arthritis, ankylosing spondylarthritis, and reactive arthritis; allergic reactions, for example, an allergic reaction associated with a disorder selected from: atopic dermatitis, pruritis, urticaria, asthma, conjunctivitis, allergic rhinitis, and anaphylaxis; CNS inflammation for example, CNS inflammation associated with a disorder selected from: Alzheimer's disease, stroke, dementia, amyotrophic lateral sclerosis, and human immunodeficiency virus; atherosclerosis; undesired immune cell activity, and inflammation associated with a disorder selected from: osteoarthritis, anaphylaxis, Behcet's disease, graft rejection, vasculitis, gout, spondylitis, viral disease, bacterial disease, lupus, inflammatory bowel disease, autoimmune hepatitis, and type 1 diabetes mellitus; age-related macular degeneration; cough; leukemia; lymphoma; CNS tumors; prostate cancer; Alzheimer's disease; stroke-induced damage; dementia; amyotrophic lateral sclerosis, and Parkinson's disease.

In some embodiments, the present methods involve use of the CB₂ agonists described herein treat acute inflammatory pain, or chronic inflammatory pain, or neuropathic pain.

Another aspect of the present disclosure relates to methods for treatment of pain in an individual comprising administering a therapeutically effective amount of a selective CB₂ agonist as described herein or a pharmaceutical compositions comprising the selective CB₂ agonists described herein to the individual in need thereof, for the treatment of a pain condition, for example, neuropathic pain (for example diabetic neuropathy pain), post-operative pain including acute post-operative pain, pain associated with osteoarthritis, chemotherapy-induced pain, pain associated with endometriosis, pain associated with interstitial cystitis, pain from migraine, non-radicular low back pain, pain associated with osteoarthritis, and other pain conditions, in accordance with the Methods and embodiments thereof described herein.

### II. Disorders of the Immune System

IIa. Autoimmune Disorders.

In some embodiments, the CB₂ receptor-mediated disorder is an autoimmune disorder. Cannabinoid receptor agonists have been shown to attenuate aberrant immune responses in autoimmune disorders, and in some cases, to provide protection to the tissue that is being inappropriately targeted by the immune system. For example, multiple sclerosis (MS) is an autoimmune disorder that results in the demyelination of neurons in the CNS. The CB₁/CB₂ receptor agonist THC significantly inhibits the severity of clinical disease in the Experimental Autoimmune Encephalomyelitis (EAE) mouse model of MS, an effect that is believed to be mediated by CB₁ on neurons and CB₂ on immune cells (Nat. Med. 13(4):492-497, 2007). Consistent with these results, CB₂-selective agonist HU-308 markedly reduces the recruitment of immature myeloid cells and T cells, microglial and infiltrating myeloid cell proliferation, and axonal loss in the EAE model (J. Biol. Chem. 283(19):13320-9, 2008). Likewise, the CB₁/CB₂ receptor agonist WIN 55212-2 significantly inhibits leukocyte rolling and adhesion in the brain in the EAE mouse model, an effect that is blocked by the CB₂-selective antagonist SR144528 but not the CB₁-selective antagonist SR141716A (Mult. Sclerosis 10(2):158-64, 2004). Accordingly, CB₂ receptor agonists find use in the treatment and/or prophylaxis of multiple sclerosis and related autoimmune demyelinating diseases, e.g. Guillan-Barré syndrome, polyradiculoneuropathy, and chronic inflammatory demyelination.

As another example, the autoimmune disease rheumatoid arthritis (RA) is a chronic, systemic inflammatory disorder of the skeletal system that principally attacks the joints to produce an inflammatory synovitis and that often progresses to destruction of the articular cartilage and ankylosis of the joints. The CB₁/CB₂ receptor agonists WIN 55212-2 and HU-210 significantly inhibit IL-1alpha-stimulated proteoglycan and collagen degradation in bovine nasal cartilage explants in vitro (J. Pharm. and Pharmacol. 58:351-358, 2006). Accordingly, CB₂ receptor agonists find use in the treatment and/or prophylaxis of autoimmune arthritic diseases, for example, rheumatoid arthritis, psoriatic arthritis, ankylosing spondylarthritis, and reactive arthritis.

IIb. Type 1 Hypersensitivity and Allergic Response.

In some embodiments, the CB₂ receptor-mediated disorder is a type 1 hypersensitivity or allergic response. Cannabinoid receptor agonists have also been shown to attenuate aberrant immune responses in allergic reactions. In type-1 (or immediate) hypersensitivity, plasma cells that have been activated by an allergen secrete IgE antibodies, which bind to Fc receptors on the surface of tissue mast cells and blood basophils and eosinophils. Repeated exposure to the same allergen results in cross-linking of the bound IgE on sensitized cells, resulting in the secretion of pharmacologically active mediators such as histamine, leukotriene and prostaglandin. These mediators are responsible for the symptoms associated with allergies, including vasodilation and increased permeability, smooth muscle spasms, and leukocyte extravasation. Topical administration of the CB₁/CB₂ receptor agonist HU-210 reduces these histamine-induced responses in human skin (Inflamm. Res. 52:238-245, 2003). Similarly, subcutaneous injection of CB₁/CB₂ receptor agonist THC or increased levels of endogenous cannabinoids reduces cutaneous inflammation and the pruritus (itch) associated with it in a mouse model for allergic contact dermatitis. (Science, 316(5830), 1494-1497, 2007). Accordingly, CB₂ receptor agonists find use in the treatment of allergic reactions including atopic dermatitis (pruritus/itch), urticaria (hives), asthma, conjunctivitis, allergic rhinitis (hay fever), and anaphylaxis.

IIc. Conditions Associated with CNS Inflammation.

In some embodiments, the CB₂ receptor-mediated disorder is a condition associated with CNS inflammation. CB₂ receptor agonists have been shown to attenuate inflammation in the CNS. For example, the administration of CB₂ receptor agonists prevents the activation of microglia in rodent models of Alzheimer's Disease (Curr. Neuropharmacol. 5(2):73-80, 2007). Likewise, the administration of CB₂ receptor agonists reduces the volume of infarcts by 30% in a rodent occlusion model of stroke (J. Cereb. Blood Flow Metab. 27:1387-96, 2007). Thus, CB₂ receptor agonists find use in the treatment and/or prophylaxis of neuropathologies associated with CNS inflammation, e.g. Alzheimer's, stroke-induced damage, dementia, ALS, and HIV.

IId. Conditions Associated with Vascular Inflammation.

In some embodiments, the CB₂ receptor-mediated disorder is a condition associated with vascular inflammation. CB₂ is expressed in macrophages and T cells in atherosclerotic plaques, and the CB₁/CB₂ receptor agonist THC reduces the progression of atherosclerosis in ApoE knockout mice, a well-studied mouse model of atherosclerosis. The CB₂-specific antagonist SR144528 completely blocks this effect in vitro and in vivo (Nature 434:782-786, 2005). Thus, CB₂ receptor agonists find use in treating atherosclerosis.

IIe. Other Disorders Associated with Aberrant or Unwanted Immune Response.

In some embodiments, the CB₂ receptor-mediated disorder is a disorder associated with aberrant or unwanted immune response. Given the expression of CB₂ on a number of different types of immune cells and the attenuating effects that CB₂ receptor agonists have been observed to have on the activities of these cells, CB₂ receptor agonists are useful for the treatment and/or prophylaxis of other disorders wherein undesired immune cell activity and/or inflammation is observed. Such exemplary disorders include osteoarthritis, anaphylaxis, Behcet's disease, graft rejection, vasculitis, gout, spondylitis, viral and bacterial diseases, e.g. AIDS, and meningitis; and other autoimmune disorders such as lupus, e.g. systemic lupus erythematosus; inflammatory bowel disease, e.g. Crohn's disease, ulcerative colitis; psoriasis; autoimmune hepatitis; and type 1 diabetes mellitus.

### III. Bone and Joint Diseases

IIIa. Osteoporosis.

In some embodiments, the CB₂ receptor-mediated disorder is osteoporosis. CB₂ is expressed in osteoblasts, osteocytes, and osteoclasts. Osteoblasts make new bone, whereas osteoclasts degrade it. The CB₂-specific agonist HU-308 enhances endocortical osteoblast numbers and activity while simultaneously inhibiting proliferation of osteoclast precursors in bone marrow-derived osteoblasts/stromal cells in vitro, and attenuates ovariectomy-induced bone loss and stimulates cortical thickness by stimulating endocortical bone formation and suppressing osteoclast number in vivo (PNAS 103(3):696-701, 2006). Thus, CB₂ receptor agonists are useful for the treatment and/or prophylaxis of disease wherein bone density is decreased, such as osteoporosis.

IIIb. Arthritis.

In some embodiments, the CB₂ receptor-mediated disorder is arthritis. CB₂ receptor agonists are useful for the treatment and/or prophylaxis of autoimmune arthritic diseases, for example, rheumatoid arthritis, psoriatic arthritis, ankylosing spondylarthritis, and reactive arthritis, and for the treatment and/or prophylaxis of inflammation associated with osteoarthritis.

### IV. Eye Disease

In some embodiments, the CB₂ receptor-mediated disorder is an eye disease. Retinal pigment epithelial (RPE) cells provide trophic support to photoreceptor cells in the eye, and RPE cell death has been shown to be a major contributor to age-related macular degeneration (AMD). The CB₁/CB₂ receptor agonist CP55,940 significantly protects RPE cells from oxidative damage, and the CB₂ receptor agonist JWHO15 provides comparable protection (Mol. Vis. 15:1243-51, 2009). Accordingly, CB₂ receptor agonists find use in preventing the onset or progression of vision loss associated with AMD.

### V. Cough

In some embodiments, the CB₂ receptor-mediated disorder is cough. The cough reflex is predominantly under the control of two classes of sensory afferent nerve fibers, the myelinated A-delta fibers and the non-myelinated C-fibers, the activation of which (i.e. depolarization) elicits cough via the vagus nerve afferent pathway. The CB₁/CB₂ receptor agonist CP55,940 reduces capsaicin-, PGE2-, and hypertonic saline-induced depolarization of guinea pig and human vagus nerve preparations in vitro (British J. Pharma. 140:261-8, 2003). The CB₂-selective agonist JWH133 also reduces capsaicin-, PGE2-, and hypertonic saline-induced depolarization of guinea pig and human vagus nerve preparations in vitro, and administration of CB₂-selective agonist JWH133 prior to exposure to the tussive agent citric acid significantly reduces cough in conscious guinea-pigs (British J. Pharma. 140:261-8, 2003). The CB₁/CB₂ receptor agonists WIN 55212-2 produces a dose-dependent inhibition of the number of capsaicin-induced coughs in mice (Eur. J. Pharmacol. 474:269-272, 2003). The CB₁/CB₂ receptor agonist anandamide produces a dose-dependent inhibition of the number of capsaicin-induced coughs in guinea pigs (Nature 408:96-101, 2000). Thus, the CB₂ receptor plays an important role in mediating the antitussive effect of cannabinoids, and CB₂ receptor agonists are useful in the treatment and/or prophylaxis of cough.

### VI. Cancer

In some embodiments, the CB₂ receptor-mediated disorder is cancer. A number of human leukemia and lymphoma cell lines, including Jurkat, Molt-4 and Sup-T1, express CB₂ receptors and not CB₁ receptors, and agonists of the CB₂ receptor induce apoptosis in these and primary acute lymphoblastic leukemia (ALL) cells (US2004/0259936). Similarly, the CB₂ receptor is expressed on glioblastoma cell lines and treatment with agonists of CB₂ induces apoptosis of these cells in vitro (J. Neurosci. Res. 86(14):3212-20, 2008). Accordingly, CB₂ receptor agonists are useful in attenuating the growth of a malignancy of the immune system, for example, leukemias, lymphomas, and solid tumors of the glial lineage.

As discussed herein, CB₁/CB₂ receptor agonists are also useful in providing relief from pain associated with cancer (GW Pharmaceuticals press releases dated Jan. 19, 2005; Jun. 19, 2007).

CB₂-mediated signaling is involved in the in vivo and in vitro growth inhibition of prostate cancer cells, which suggests that CB₂ receptor agonists have potential therapeutic interest in the management of prostate cancer. (British Journal of Cancer advance online publication 18 Aug. 2009; doi: 10.1038/sj.bjc. 6605248).

### VII. Regenerative Medicine

In some embodiments, the CB₂ receptor-mediated disorder is a degenerative disorder. Agonists of CB₂ modulate the expansion of the progenitor pool of neurons in the CNS. CB₂ antagonists inhibit the proliferation of cultured neural stem cells and the proliferation of progenitor cells in the SVZ of young animals, whereas CB₂-selective agonists stimulate progenitor cell proliferation in vivo, with this effect being more pronounced in older animals (Mol. Cell Neurosci. 38(4):526-36, 2008). Thus, agonists of CB₂ are useful in regenerative medicine, for example to promote the expansion of progenitor cells for the replacement of neurons lost during injury or disease, such as Alzheimer's Disease, stroke-induced damage, dementia, amyotrophic lateral sclerosis (ALS) and Parkinson's Disease.

### VIII. Fibrosis

In some embodiments, the CB₂ receptor-mediated disorder is fibrosis or a condition related thereto. Fibrosis is the accumulation of excess extracellular matrix components in organs and/or tissues. Pirfenidone was recently approved by the U.S. FDA for the treatment of idiopathic pulmonary fibrosis (IPF). However, very few treatments exist for other fibrotic conditions. There is a serious unmet need for such treatments.

As discussed herein, the CB₂ signaling pathway has been identified as an anti-fibrogenic pathway. CB₂ receptor agonists are useful for the treatment or prevention of fibrosis. In some embodiments, the compounds and/or pharmaceutical compositions described herein are useful for the treatment or prevention of fibrosis or condition related thereto. In some embodiments, the compounds/agonists described herein are useful for the treatment or prevention of fibrosis associated with a disease, disorder, and/or condition.

In some embodiments, the fibrosis is a chronic fibroproliferative disease. In some embodiments, the fibrosis occurs systemically. For example, in some embodiments, the fibrosis is systemic sclerosis, cystic fibrosis, nephrogenic systemic fibrosis, chronic graft versus host disease, or atherosclerosis. In some embodiments, the fibrosis is isolated to a particular organ or tissue.

In some embodiments, the fibrosis is scleroderma. In some embodiments, the fibrosis is limited scleroderma. In some embodiments, the fibrosis is limited cutaneous scleroderma. In some embodiments, the fibrosis is diffuse scleroderma. In some embodiments, the fibrosis is diffuse cutaneous scleroderma.

In some embodiments, the fibrosis occurs in the liver. In some embodiments, the fibrosis is associated with nonalcoholic steatohepatitis (NASH), alcoholic steatohepatitis (ASH), idiopathic portal hypertension, hepatic fibrosis (including congenital hepatic fibrosis), viral hepatitis B or C, autoimmune hepatitis, primary sclerosing cholangitis, primary biliary cirrhosis, or idiopathic portal hypertension. In some embodiments, the fibrosis is associated with liver steatosis. In some embodiments, the fibrosis is liver fibrosis (or “hepatic fibrosis”). In some embodiments, the fibrosis is cirrhosis. In some embodiments, the fibrosis is associated with alcoholic liver disease.

In some embodiments, the fibrosis occurs in the kidneys. In some embodiments, the fibrosis is associated with focal segmental glomerulosclerosis (FSGS), glomerulonephritis, IgA nephropathy, diabetic nephropathy, transplant nephropathy, chronic allograft nephropathy, lupus nephritis, or unilateral ureteral obstruction-induced renal fibrosis. In some embodiments, the fibrosis is renal fibrosis.

In some embodiments, the fibrosis occurs in the lungs. In some embodiments, the fibrosis is associated with asthma, cystic fibrosis, chronic obstructive pulmonary disease (COPD), pulmonary arterial hypertension, acute respiratory distress syndrome (ARDS), or scleroderma lung disease. In some embodiments, the fibrosis is progressive massive fibrosis. In some embodiments, the fibrosis is pulmonary fibrosis (such as idiopathic pulmonary fibrosis). In some embodiments, the fibrosis is renal fibrosis characterized by tubulointerstitial fibrosis and glomerulosclerosis.

In some embodiments, the fibrosis occurs in the eyes. In some embodiments, the fibrosis is associated with age-related macular degeneration (AMD), glaucoma, diabetic macular edema, diabetic retinopathy, or dry eye disease.

In some embodiments, the fibrosis occurs in the heart. In some embodiments, the fibrosis is associated with heart failure, atherosclerosis, endomyocardial fibrosis, myocardial infarction, or atrial fibrosis. In some embodiments, the fibrosis is associated with congestive heart failure. In some embodiments, the fibrosis is cardiac fibrosis.

In some embodiments, the fibrosis occurs in soft tissue, bone marrow, skin, or peritoneum. In some embodiments, the fibrosis is mediastinal fibrosis, myelofibrosis (e.g., idiopathic- or drug-induced myelofibrosis), retroperitoneal fibrosis, nephrogenic systemic fibrosis, systemic sclerosis, or discoid lupus erythematosus. In some embodiments, the fibrosis occurs in the skin. In some embodiments, the fibrosis is associated with scleroderma, keloids, hypertrophic scarring, eosinophilic fasciitis, or dermatomyositis. In some embodiments, the fibrosis is skin scarring. In some embodiments, the compounds described herein are useful for reducing the severity of a scar. In some embodiments, the compounds described herein are useful for wound repair.

In some embodiments, the fibrosis occurs in a joint or joints. In some embodiments, the fibrosis occurs in the hands and/or fingers. In some embodiments, the fibrosis is athrofibrosis, Dupuytren's contracture, or adhesive capsulitis.

In some embodiments, the fibrosis occurs in the intestine. In some embodiments, the fibrosis is associated with Crohn's Disease.

In some embodiments, the fibrosis occurs in the penis. In some embodiments, the fibrosis is associated with Peyronie's disease.

In some embodiments, the fibrosis is the result of injury, surgery, or radiation. In some embodiments, the fibrosis is burn-induced. For example, in some embodiments, the fibrosis is burn-induced scarring and/or contraction. In some embodiments, the fibrosis is chemotherapy-induced (e.g., bleomycin-induced) pulmonary fibrosis. In some embodiments, the fibrosis is scarring following trabeculectomy in an individual with glaucoma. In some embodiments, the fibrosis is the result of an infection.

In some embodiments, the compounds described herein are useful for the treatment of idiopathic pulmonary fibrosis (“IPF”). In some embodiments, an individual in need of treatment has received a clinical and radiographic diagnosis of IPF. In some embodiments, an individual in need of treatment has undergone a surgical lung biopsy. In some embodiments, an individual in need of treatment has a percent predicted forced vital capacity (% FVC) greater than or equal to 50% at baseline. In some embodiments, an individual in need of treatment has a percent predicted diffusing capacity of the lungs for carbon monoxide (% DLCO) greater than or equal to 30% or 35%.

### IX. Interstitial Cystitis/Painful Bladder Syndrome

In some embodiments, the CB₂ receptor-mediated disorder is interstitial cystitis. Interstitial cystitis (also known as painful bladder syndrome) is a chronic inflammatory condition of the bladder associated with urinary urgency, urinary frequency, and nocturia. CB₂ receptors have been reported to be present in the bladder and its associated innervation, and CB₂ receptors are upregulated in bladder after acute or chronic inflammation. CB₂ receptors have therefore been suggested as a target for pharmacological treatment of bladder inflammation and associated pain. Neurosci Lett 445(1):130-134, 2008. Further, lipopolysaccharide (LPS)-induced bladder inflammation has been shown to increase expression of bladder CB₂ (but not CB₁) mRNA, and CB₂ receptor agonist JWHO15 has been shown to antagonize LPS-induced bladder inflammation (Tambaro et al., Eur J Pharmacol 2014). Accordingly, CB₂ receptor agonists find use in the treatment of interstitial cystitis.

In some embodiments, an individual is diagnosed and/or assessed for a disease, condition, or disorder disclosed above based on information from an imaging technique. For example, in some embodiments, an individual is diagnosed and/or assessed based on an ultrasound (e.g., FibroScan), CT (e.g., high resolution CT (HRCT)), or MRI scan. In some embodiments, an individual is diagnosed and/or assessed based on a pulmonary function test. For example, in some embodiments, a change in percent predicted forced volume vital capacity (FVC) from baseline to a defined endpoint is assessed. In some embodiments, an individual is diagnosed and/or assessed for pain based on the Western Ontario and McMasters Universities Osteoarthritis (WOMAC) Index.

Provided herein are CB₂ receptor agonist compounds useful for the treatment of a CB₂ receptor-mediated disorder. Also provided are methods for the treatment of a CB₂ receptor-mediated disorder in an individual in need thereof, comprising administering to the individual a therapeutically effective amount of a compound described herein. Also provided are compounds useful for the treatment of pain (e.g., acute pain, inflammatory pain, neuropathic pain, chemotherapy-induced pain, abdominal pain in inflammatory bowel disease, interstitial cystitis, migraine, and low back pain). Also provided are compounds useful for the treatment of osteoarthritis. Also provided are compounds useful for the treatment of a liver disease selected from liver fibrosis, primary biliary cirrhosis, and nonalcoholic steatohepatitis. In some embodiments, the liver disease is liver fibrosis. In some embodiments, the liver disease is primary biliary cirrhosis. In some embodiments, the liver disease is nonalcoholic steatohepatitis. Also provided are compounds useful for the treatment of bone and joint pain. Also provided are compounds useful for the treatment of bone pain. Also provided are compounds useful for the treatment of joint pain. Also provided are compounds useful for the treatment of pain associated with osteoarthritis. Also provided are compounds useful for the treatment of osteoporosis. Also provided are compounds useful for the treatment of hyperalgesia. Also provided are compounds useful for the treatment of allodynia. Also provided are compounds useful for the treatment of inflammatory pain. Also provided are compounds useful for the treatment of inflammatory hyperalgesia. Also provided are compounds useful for the treatment of neuropathic pain. Also provided are compounds useful for the treatment of neuropathic hyperalgesia. Also provided are compounds useful for the treatment of acute nociception. Also provided are compounds useful for the treatment of muscle pain. Also provided are compounds useful for the treatment of dental pain. Also provided are compounds useful for the treatment of migraine and other headache pain. Also provided are compounds useful for the treatment of pain that occurs as an adverse effect of therapeutics. Also provided are compounds useful for the treatment of pain associated with a disorder selected from: cancer, multiple sclerosis, allergic reactions, nephritic syndrome, scleroderma, thyroiditis, diabetic neuropathy, fibromyalgia, HIV related-neuropathy, sciatica, and autoimmune conditions. Also provided are compounds useful for the treatment of multiple sclerosis-associated spasticity. Also provided are compounds useful for the treatment of autoimmune disorders. Also provided are compounds useful for the treatment of an autoimmune disorder selected from the group consisting of: multiple sclerosis, Guillan-Barré syndrome, polyradiculoneuropathy, chronic inflammatory demyelination, rheumatoid arthritis, psoriatic arthritis, ankylosing spondylarthritis, and reactive arthritis. Also provided are compounds useful for the treatment of allergic reactions. Also provided are compounds useful for the treatment of an allergic reaction associated with a disorder selected from: atopic dermatitis, pruritis, urticaria, asthma, conjunctivitis, allergic rhinitis, and anaphylaxis. Also provided are compounds useful for the treatment of CNS inflammation. Also provided are compounds useful for the treatment of CNS inflammation associated with a disorder selected from: Alzheimer's disease, stroke, dementia, amyotrophic lateral sclerosis, and human immunodeficiency virus. Also provided are compounds useful for the treatment of atherosclerosis. Also provided are compounds useful for the treatment of undesired immune cell activity and inflammation associated with a disorder selected from: osteoarthritis, anaphylaxis, Behcet's disease, graft rejection, vasculitis, gout, spondylitis, viral disease, bacterial disease, lupus, inflammatory bowel disease, autoimmune hepatitis, and type 1 diabetes mellitus. Also provided are compounds useful for the treatment of age-related macular degeneration. Also provided are compounds useful for the treatment of cough. Also provided are compounds useful for the treatment of leukemia. Also provided are compounds useful for the treatment of lymphoma. Also provided are compounds useful for the treatment of CNS tumors. Also provided are compounds useful for the treatment of prostate cancer. Also provided are compounds useful for the treatment of Alzheimer's disease. Also provided are compounds useful for the treatment of stroke-induced damage. Also provided are compounds useful for the treatment of dementia. Also provided are compounds useful for the treatment of amyotrophic lateral sclerosis. Also provided are compounds useful for the treatment of Parkinson's disease.

In some embodiments, the disorder is a CB₂ receptor-mediated disorder. In some embodiments, the CB₂ receptor-mediated disorder is pain or a condition related thereto. In some embodiments, the CB₂ receptor-mediated disorder is osteoarthritis. In some embodiments, the CB₂ receptor-mediated disorder is fibrosis or a condition related thereto. In some embodiments, the CB₂ receptor-mediated disorder is liver fibrosis. In some embodiments, the CB₂ receptor-mediated disorder is primary biliary cirrhosis. In some embodiments, the CB₂ receptor-mediated disorder is nonalcoholic steatohepatitis. In some embodiments, the CB₂ receptor-mediated disorder is diabetic neuropathy. In some embodiments, the CB₂ receptor-mediated disorder is interstitial cystitis. In some embodiments, the CB₂ receptor-mediated disorder is pain associated with interstitial cystitis. In some embodiments, the CB₂ receptor-mediated disorder is endometriosis. In some embodiments, the CB₂ receptor-mediated disorder is pain associated with endometriosis.

### Methods of Treatment

The CB₂ receptor agonist compounds described herein can be used in methods of treating a human subject in need of treatment with a CB₂ receptor agonist compound. In certain embodiments, the CB₂ receptor agonist compound is a selective CB₂ compound. For example, in certain embodiments, the CB₂ receptor agonist compound has a >1,000-fold selectivity for the human CB₂ receptor versus the human CB₁ receptor. In one embodiment, the CB₂ receptor agonist compound is APD371.

In certain instances, the human subject in need of treatment with a CB₂ receptor agonist compound is an elderly subject. For example, the subject may be 60 years of age or older, 65 years of age or older, 70 years of age or older, 75 years of age or older, 80 years of age or older, 85 years of age or older, 90 years of age or older, or 95 years of age or older.

In certain embodiments, the human subject in need of treatment with a CB₂ receptor agonist compound has chronic pain. In certain embodiments, the human subject in need of treatment with a CB₂ receptor agonist compound has acute pain. In some embodiments, the human subject in need of treatment with a CB₂ receptor agonist compound has acute inflammatory pain. In some embodiments, the human subject in need of treatment with a CB₂ receptor agonist compound has chronic inflammatory pain. In yet other embodiments, the human subject in need of treatment with a CB₂ receptor agonist compound has neuropathic pain. In other embodiments, the human subject in need of treatment with a CB₂ receptor agonist compound has acute neuropathic pain. In yet other embodiments, the human subject in need of treatment with a CB₂ receptor agonist compound has chronic neuropathic pain. In other embodiments, the human subject in need of treatment with a CB₂ receptor agonist compound has pain associated with osteoarthritis. In other embodiments, the human subject in need of treatment with a CB₂ receptor agonist compound has acute post-operative pain. In certain embodiments, the human subject in need of treatment with a CB₂ receptor agonist compound has fibrosis (e.g., lung fibrosis, liver fibrosis, renal fibrosis). In certain embodiments, the human subject in need of treatment with a CB₂ receptor agonist compound presents with primary biliary cirrhosis, nonalcoholic steatohepatitis, endometriosis, or interstitial cystitis. In all of these embodiments, the CB₂ receptor agonist compound that is used for treating the subject can be APD371.

As described in Example 2, it was unexpectedly found that human subjects administered the CB₂ receptor agonist, APD371, exhibited reductions in blood pressure and heart rate.

Thus, in certain embodiments of the methods of treatment of the present disclosure, the human subject is assessed for the risk of, or presence of, a low heart rate. The determination of heart rate of a subject can occur prior to, during, and/or after administration of the CB₂ receptor agonist compound (e.g., APD371) to the subject. In certain instances, heart rate is measured both before and after administration of the CB₂ receptor agonist compound. In certain cases, the subject is evaluated for heart rate at, about, or at least about, 0.1, 0.2, 0.3, 0.4, 0.5, 0.6, 0.7, 0.8, 0.9, 1, 1.5, 2, 2.5, 3, 3.5, 4, 4.5, 5, 5.5, or 6 hours before and/or following administration of the CB₂ receptor agonist compound (e.g., APD371). In certain instances, the heart rate is a resting heart rate. In other cases, the heart rate is a supine heart rate. In some other cases, the heart rate is a standing heart rate. Generally, the normal resting heart rate of a human subject is between about 60 to about 100 beats per minute (e.g., 60 to 100 bpm). Accordingly, in one embodiment a low heart rate is a resting heart rate less than 60 beats per minute. In another embodiment, a low heart rate is a resting heart rate less than or equal to 55 beats per minute. In another embodiment, a low heart rate is a resting heart rate less than or equal to 50 beats per minute. In another embodiment, a low heart rate is a resting heart rate less than or equal to 45 beats per minute. In another embodiment, a low heart rate is a resting heart rate less than or equal to 40 beats per minute. In another embodiment, a low heart rate is a resting heart rate that is 40 to 59 beats per minute. In another embodiment, a low heart rate is a resting heart rate that is 40 to 55 beats per minute. In another embodiment, a low heart rate is a resting heart rate that is 40 to 50 beats per minute. In another embodiment, a low heart rate is a resting heart rate that is 50 to 59 beats per minute. In another embodiment, a low heart rate is a resting heart rate that is 50 to 55 beats per minute. In some instances, a human subject may have a risk of low heart rate if the human subject has previously had bradycardia.

In some embodiments of the methods of treatment encompassed by the present disclosure, the human subject is assessed for the risk of, or presence of, low blood pressure. The determination of blood pressure of a subject can occur prior to, during, and/or after administration of the CB₂ receptor agonist compound (e.g., APD371) to the subject. In certain instances, blood pressure is measured both before and after administration of the CB₂ receptor agonist compound. In certain instances, blood pressure is measured after administration of the CB₂ receptor agonist compound. In certain cases, the subject is evaluated for blood pressure at, about, or at least about, 0.1, 0.2, 0.3, 0.4, 0.5, 0.6, 0.7, 0.8, 0.9, 1, 1.5, 2, 2.5, 3, 3.5, 4, 4.5, 5, 5.5, or 6 hours before and/or following administration of the CB₂ receptor agonist compound (e.g., APD371). Blood pressure is typically described in terms of systolic blood pressure and diastolic blood pressure (systolic/diastolic). The systolic blood pressure can be: a resting systolic blood pressure, a supine systolic blood pressure, or a standing systolic blood pressure. The diastolic blood pressure can be: a resting diastolic blood pressure, a supine diastolic blood pressure, or a standing diastolic blood pressure. Generally, normal blood pressure is about 120/80 mmHg. In other words, the normal blood pressure is a systolic blood pressure that is about 120 mmHg, and a diastolic blood pressure that is about 80 mmHg. Generally, hypotension is a blood pressure less than, or less than about, 90/60 mmHg. Accordingly, in some embodiments, a low blood pressure is a systolic blood pressure that is less than, or less than about, 90 mmHg. In some embodiments, a low blood pressure is a diastolic blood pressure that is less than, or less than about, 60 mmHg. In some embodiments, a low blood pressure reading is 89/59 mmHg or lower (i.e., a systolic blood pressure that is 89 or lower, and a diastolic blood pressure that is 59 or lower). In some embodiments, a low blood pressure is a blood pressure reading of 85/55 mmHg or lower. In some embodiments, a low blood pressure is a blood pressure reading of 80/50 mmHg or lower. In some embodiments, a low blood pressure is a systolic blood pressure of less than, or less than about, 120, 115, 110, 105, 100, 95, 90, 85, 80, 75, or 70 mmHg. In some embodiments, a low blood pressure is a diastolic blood pressure of less than, or less than about, 80, 75, 70, 65, 60, 55, or 50 mmHg. In certain embodiments a subject is determined to have a low blood pressure if her/his systolic blood pressure is 89, 88, 87, 86, 85, 84, 83, 82, 81, 80, 79, 78, 77, 76, 75, 74, 73, 72, 71, 70 mmHg; and/or if her/his diastolic blood pressure is 59, 58, 57, 56, 55, 54, 53, 52, 51, 50, 49, or 48 mmHg. In certain embodiments a subject is determined to have low blood pressure if she/he has a systolic blood pressure of 75 to 89, 75 to 85, or 75 to 80 mmHg; and/or she/he has a diastolic blood pressure of 45 to 59, 45 to 57, 45 to 56, 45 to 55, 45 to 54, 48 to 59, 48 to 57, 48 to 55 mmHg. In some instances, a human subject may have a risk of low blood pressure if the human subject has previously had orthostatic hypotension. Orthostatic hypotension is defined as a decrease in systolic blood pressure of 20 mmHg or a decrease in diastolic blood pressure of 10 mmHg within three minutes of standing compared with blood pressure from the sitting or supine position. Orthostatic hypotension may also be diagnosed, e.g., by head-up tilt-table testing at an angle of at least 60 degrees.

A human subject may have, or be at risk of having, low blood pressure and/or low heart rate if the human subject: has had or is taking prolonged bed rest; is within the first 24 weeks of pregnancy; has suffered decreases in blood volume (e.g., as a result of trauma, severe internal bleeding, dehydration); is taking certain types of medications (e.g., anti-hypertensive medications, diuretics, beta-blockers, drugs for Parkinson's disease, tricyclic antidepressants, erectile dysfunction drugs alone or in combination with nitroglycerine, digoxin, antiarrhythmics); is taking narcotics or alcohol; if the subject has heart conditions that lead to low heart rate (bradycardia); has had a heart attack; has problems with heart valve(s); has coronary artery disease; has endocarditis; has myocarditis; has hypothyroidism; has parathyroid disease; has Addison's disease; has low blood sugar; has diabetes; has septic shock; has neutrally mediated hypotension; has anemia; has an electrolyte imbalance (e.g., high levels of potassium in the blood); or has a deficiency in vitamin B-12 and/or folic acid.

According to the methods of this disclosure, the human subject is administered a therapeutically effective amount of a selective CB₂ receptor agonist (e.g., APD371) if the human subject does not have a risk of, or the presence of, a low heart rate and/or low blood pressure. In contrast, the human subject is not administered a selective CB₂ receptor agonist (e.g., APD371) if the human subject does not have a risk of, or the presence of, a low heart rate and/or low blood pressure.

In certain embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, orthostatic hypotension. In certain embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, neutrally mediated hypotension. In some embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, Addison's disease. In certain embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, diabetes. In other embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, anemia. In other embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject does not have, or is not at risk of developing, bradycardia. In yet other embodiments, this disclosure provides methods for treating a human subject in need of treatment with a selective CB₂ receptor agonist (e.g., APD371), comprising administering a therapeutically effective amount of the selective CB₂ receptor agonist to the human subject if the human subject is not taking, or has not recently been taking (e.g., within 6 months, within 5 months, within 3 months, within 2 months, within a month, within 3 weeks, within 2 weeks, within 1 week, within 6 days, within 5 days, within 4 days, within 3 days, within 2 days, with a day of the proposed administration of the selective CB₂ receptor agonist (e.g., APD371)), a medication selected from the group consisting of: an anti-hypertensive, a diuretic, a beta-blocker, a drug for Parkinson's disease, a tricyclic antidepressant, an erectile dysfunction drug, nitroglycerine, a CYP inhibitor, a drug that increases exposure of APD371, a drug that slows the metabolism of APD371, a drug that competes with APD371 for protein binding, a vasodilator, a drug that causes QT prolongation, an alpha blocker, and a nitrate.

In some embodiments, the CB₂ receptor agonist (e.g., APD371) is administered orally to the subject. In some embodiments, the CB₂ receptor agonist (e.g., APD371) is administered to the subject as a tablet or capsule. In certain embodiments, a therapeutically effective amount of the CB₂ receptor agonist (e.g., APD371) is a dose of 50 mg. In other embodiments, a therapeutically effective amount of the CB₂ receptor agonist (e.g., APD371) is a dose of 60 mg. In other embodiments, a therapeutically effective amount of the CB₂ receptor agonist (e.g., APD371) is a dose of 75 mg. In other embodiments, a therapeutically effective amount of the CB₂ receptor agonist (e.g., APD371) is a dose of 100 mg. In other embodiments, a therapeutically effective amount of the CB₂ receptor agonist (e.g., APD371) is a dose of 125 mg. In some embodiments, a therapeutically effective amount of the CB₂ receptor agonist (e.g., APD371) is a dose of 150 mg. In yet other embodiments, a therapeutically effective amount of the CB₂ receptor agonist (e.g., APD371) is a dose of 200 mg. In other embodiments, a therapeutically effective amount of the CB₂ receptor agonist (e.g., APD371) is a dose of 225 mg. In yet other embodiments, a therapeutically effective amount of the CB₂ receptor agonist (e.g., APD371) is a dose of 250 mg. In other embodiments, a therapeutically effective amount of the CB₂ receptor agonist (e.g., APD371) is a dose of 275 mg. In other embodiments, a therapeutically effective amount of the CB₂ receptor agonist (e.g., APD371) is a dose of 300 mg. In some other embodiments, a therapeutically effective amount of the CB₂ receptor agonist (e.g., APD371) is a dose of 350 mg. In yet other embodiments, a therapeutically effective amount of the CB₂ receptor agonist (e.g., APD371) is a dose of 400 mg. In certain embodiments, the dose of the CB₂ receptor agonist is administered at a frequency of: once a day, twice a day, three times a day, or four times a day. The duration of treatment with the CB₂ receptor agonist (e.g., APD371) can be as long as needed to obtain a therapeutic effect without the occurrence of intolerable or persistent adverse events.

Sometimes, the subject is already being administered a CB₂ receptor agonist compound (e.g., APD371). In such instances, the subject is evaluated for his/her heart rate and/or blood pressure after administration of the CB₂ receptor agonist compound (e.g., APD371). Methods of measuring and evaluating heart rate and/or blood pressure are well-known in the art. If the subject does not have a low heart rate and/or low blood pressure, the administration of the CB₂ receptor agonist compound (e.g., APD371) can be continued. If, however, the subject is determined to have a low heart rate and/or low blood pressure after administration of the CB₂ receptor agonist compound, the administration of the CB₂ receptor agonist compound (e.g., APD371) should either be discontinued until the subject shows improvement of these vital signs (e.g., an increase in the heart rate and/or blood pressure to, or close to, the normal levels), or treatment may be continued at a lower dose. Regarding treatment at a lower dose, if the subject was being administered 400 mg of ADP371 before the evaluation of heart rate and/or blood pressure and the finding of a low heart rate and/or blood pressure, the lower dose of APD371 could be, e.g., 300 mg, 275 mg, 250 mg, 200 mg, 175 mg, 150 mg, 125 mg, 100 mg, 75 mg, or 50 mg. If the subject was being administered 200 mg of ADP371 before the evaluation of heart rate and/or blood pressure, the lower dose could be, e.g., 175 mg, 150 mg, 125 mg, 100 mg, 75 mg, or 50 mg. The heart rate and/or blood pressure can be monitored during the continued treatment on lower dosages and the dose can be further lowered if the subject continues to show a low heart rate and/or low blood pressure at the lower dose. Thus if a subject shows a low heart rate and/or blood pressure after administration with 400 mg of APD371, the treatment can be interrupted until the subject shows improvement in these vital signs and treatment can be continued at a lower dose (e.g., 200 mg). If the subject displays low blood pressure and/or heart rate after administration of the 200 mg dose, treatment can be interrupted until the subject shows improvement in these vital signs and treatment can be continued at a lower dose (e.g., 100 mg). If the subject still exhibits low blood pressure and/or heart rate after administration of the 100 mg dose, treatment can be interrupted until the subject shows improvement in these vital signs and treatment can be continued at a lower dose (e.g., 50 mg).

In some embodiments, low blood pressure is a systolic blood pressure <90 mmHg and/or diastolic blood pressure <50 mmHg, or a systolic blood pressure <95 mmHg, and/or a diastolic blood pressure <60 mmHg, at least one of which represents at least a 10 mmHg change from the lowest pre-dose value. In some embodiments, the pre-dose values are two different measurements at least 10 minutes, but not more than 24 hours, apart. In some embodiments, low heart rate is a heart rate of <50 bpm that is at least a 10 bpm reduction from the lowest pre-dose value. In some embodiments, the pre-dose values are taken on different occasions at least 10 minutes apart within the first 24 hours post-dosing.

The disclosure also provides methods for selecting subjects for treatment with a CB₂ receptor agonist compound. The subject can be selected for treatment based on a prior or concurrent determination that the subject does not have the risk of, or presence of, a low heart rate and/or low blood pressure. The determination can be performed by measuring vital signs. Such a subject can be administered a therapeutically effective amount of a CB₂ receptor agonist compound (e.g., APD371). Subjects that have the risk of, or presence of, a low heart rate and/or low blood pressure are either not to be treated with a CB₂ receptor agonist compound, or to be treated with a lower dose than subjects who do not have the risk of, or presence of, a low heart rate and/or low blood pressure. In certain instances, the lower dose is 80%, 75%, 70%, 65%, 60%, 55%, 50%, 45%, 40%, 35%, 30%, 25%, 20%, 15%, or 10% of the dose administered to subjects who do not have the risk of, or presence of, a low heart rate and/or low blood pressure.

In some embodiments, a contraindicated drug is not administered during the administration of APD371. In some embodiments, the dosage amount of APD371 is reduced during the administration of a contraindicated drug. In some embodiments, the dosage amount of a contraindicated drug is reduced during the administration of APD371. In some embodiments, APD371 is titrated in the presence of a contraindicated drug. For example, in some embodiments, treatment with APD371 is initiated at a lower dose (compared to a standard dose) in an individual who is administered an alpha blocker. In some embodiments, a contraindicated drug is titrated in the presence of APD371. In some embodiments, a human subject is monitored for adverse reactions (such as hypotension, syncope, and/or bradycardia) when administered a contraindicated drug. In some embodiments, the contraindicated drug is a prescription drug. In some embodiments, the contraindicated drug is a dietary and/or herbal supplement. In some embodiments, the contraindicated drug is a non-prescription drug.

In some embodiments, the concomitant use of APD371 and a CYP inhibitor is a known risk factor for an adverse event, such as bradycardia, hypotension, syncope, accidental injury and/or central nervous system (CNS) depression. In some embodiments, the concomitant use of APD371 and a CYP inhibitor is avoided.

In some embodiments, a medication known to cause hypertension and/or syncope is contraindicated with APD371. In some embodiments, an oral contraceptive is contraindicated with APD371. In some embodiments, a compound that increases flibanserin exposure is contraindicated with APD371. In some embodiments, a compound that decreases flibanserin exposure is contraindicated with APD371. In some embodiments, a nitrate is contraindicated with APD371. In some embodiments, an alpha blocker is contraindicated with APD371. In some embodiments, an anti-hypertensive is contraindicated with APD371.

In some embodiments, a CYP2C19 inhibitor (particularly a strong CYP2C19 inhibitor) is contraindicated with APD371. In some embodiments, a CYP2C9 inhibitor is contraindicated with APD371. In some embodiments, a CYP2D6 inhibitor is contraindicated with APD371. In some embodiments, a CYP3A4 inhibitor (particularly a moderate or strong CYP3A4 inhibitor) is contraindicated with APD371. In some embodiments, a CYP3A4 inducer is contraindicated with APD371. In some embodiments, a CYP1A2 inhibitor is contraindicated with APD371.

In some embodiments, alcohol use is contraindicated with APD371. In some embodiments, a human subject is assessed for the likelihood of abstaining from alcohol (e.g., taking into account the individual's current and past drinking behavior, and other pertinent social and medical history) prior to the administration of APD371. In some embodiments, a combination of multiple concomitant drugs (e.g., multiple weak CYP inhibitors, such as multiple weak CYP3A4 inhibitors) is contraindicated with APD371.

In some embodiments, APD371 is discontinued at least 1, 2, 3, 4, 5, 6, 7, 8, 9, 10, 11, 12, 13, or 14 days prior to starting the administration of a contraindicated drug, such as a CYP inhibitor. In some embodiments, a human subject is monitored (e.g., for hypotension, syncope and/or bradycardia) following discontinuation of APD371. In some embodiments, a contraindicated drug is discontinued at least 1, 2, 3, 4, 5, 6, 7, 8, 9, 10, 11, 12, 13, or 14 days prior to starting the administration of APD371.

In some embodiments, APD371 is contraindicated in a human subject with hepatic impairment. In some embodiments, APD371 is contraindicated in a human subject with renal impairment. For example, in some embodiments, a reduced dose (compared to a standard dose) of APD371 is administered to an individual with renal impairment. In some embodiments, APD371 is contraindicated in an elderly subject. In some embodiments, APD371 is contraindicated in a human subject that is, or is about, 50, 55, 60, 65, 70, 75, 80, 85, 90, or 95 years of age or older. In some embodiments, an elderly human subject is monitored (e.g., for orthostatic hypotension, dizziness, and/or lightheadedness) during the administration of APD371. In some embodiments, APD371 is contraindicated in a human subject who is exhibits poor or intermediate CYP metabolism. In some embodiments, a human subject is assessed for CYP metabolism prior to the administration of APD371. In some embodiments, a human subject who is a poor or intermediate metabolizer for a CYP is monitored during the administration of APD371. In some embodiments, a human subject who is a poor metabolizer of APD371 is monitored during the administration of APD371.

In some embodiments, APD371 is contraindicated in a human subject with bronchial asthma, overt cardiac failure, greater-than-first-degree heart block, cardiogenic shock, severe bradycardia, and/or other conditions associated with severe and prolonged hypotension.

The following are examples of the practice of the invention. They are not to be construed as limiting the scope of the invention in any way.

### EXAMPLES

### Example 1

A phase 1a randomized, double-blind, placebo-controlled single ascending dose clinical trial was performed in 56 healthy human subjects to assess the safety, tolerability, and pharmacokinetics of APD371 (Compound B). Doses of 10 mg, 20 mg, 30 mg, 60 mg, 120 mg, 250 mg, and 400 mg were administered. Subjects were in the clinic for approximately 9 days (referred to as Day −2 to Day 7). Study medication (APD371 or placebo) was administered on Day 1. Each cohort included eight human subjects, six of whom were administered APD371 and two of whom were administered placebo.

Vital signs were measured for blood pressure and heart rate in the supine position after resting for five minutes. 12-lead electrocardiograms were used to measure RR, PR, QRS, QT, QTc, QTcB, and QTcF in the recumbent position after resting for 10 minutes. 5-lead electrocardiograms were used for continuous telemetry, with measurements taken during the last five minutes of a resting period of 14 minutes±1 minute in the supine position. Baseline measurements were taken prior to dosing.

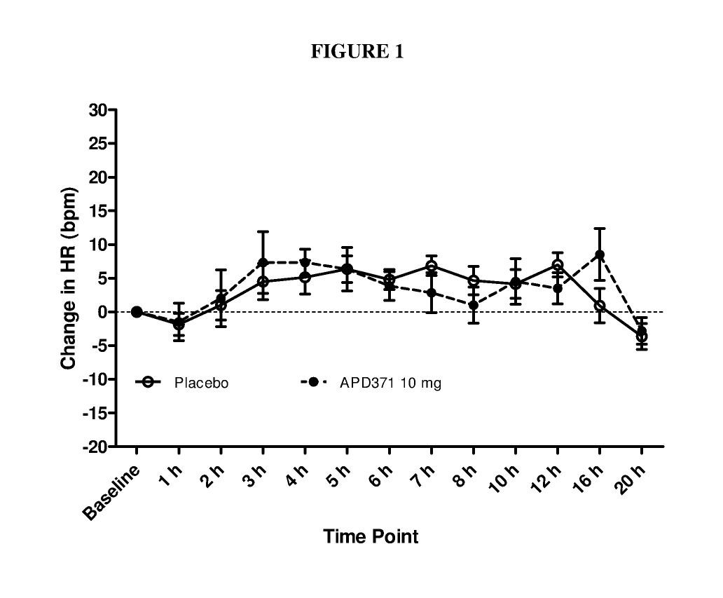

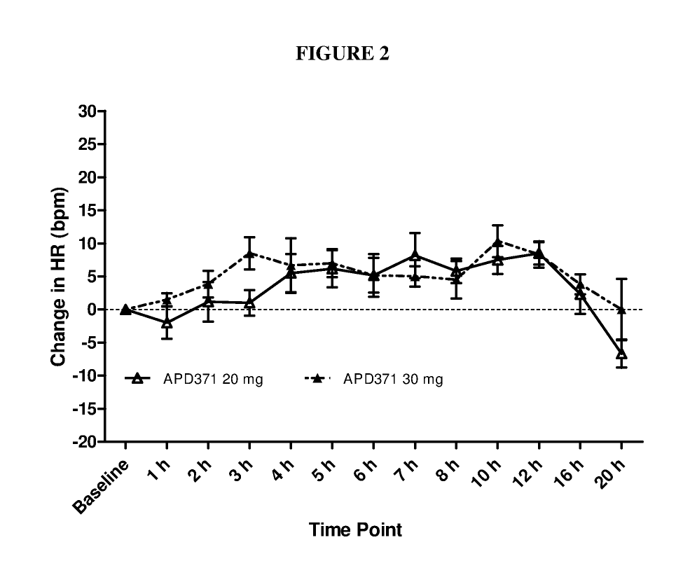

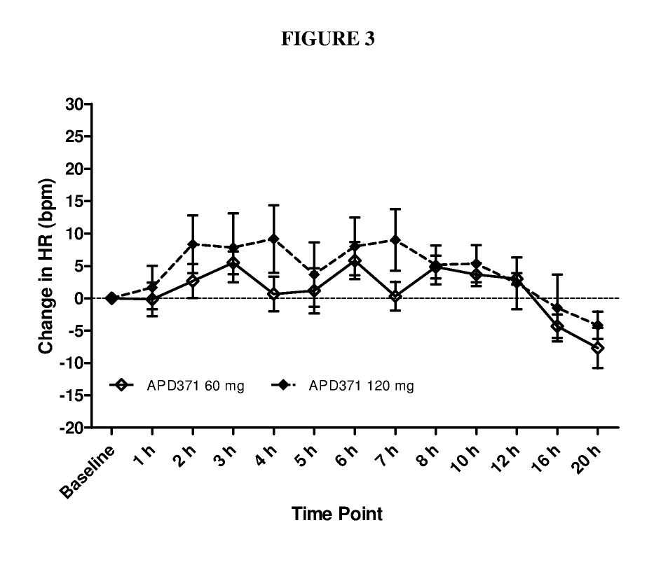

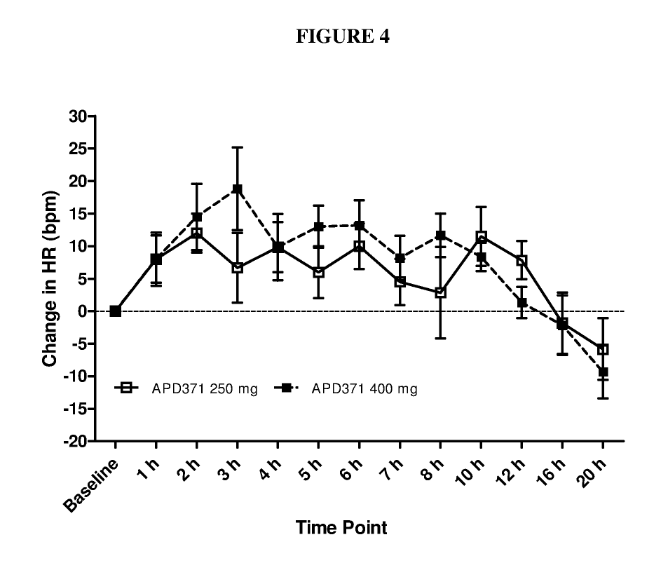

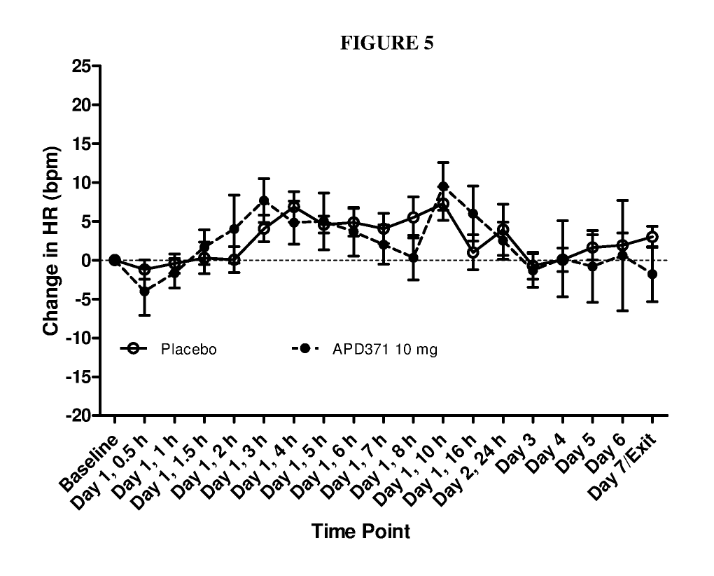

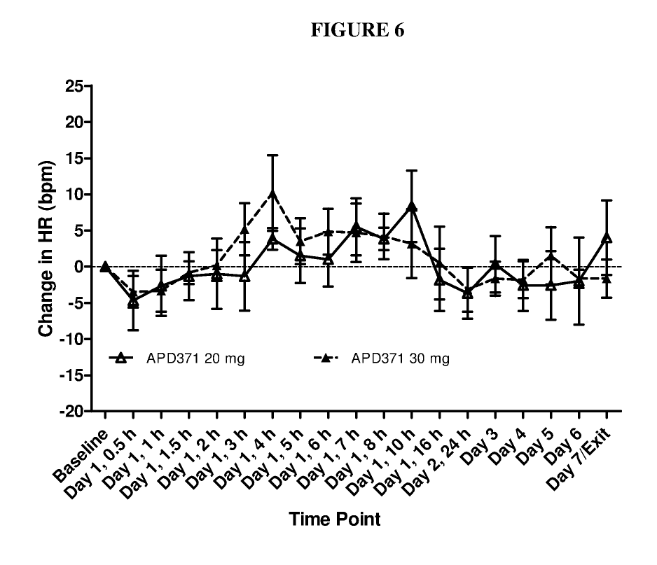

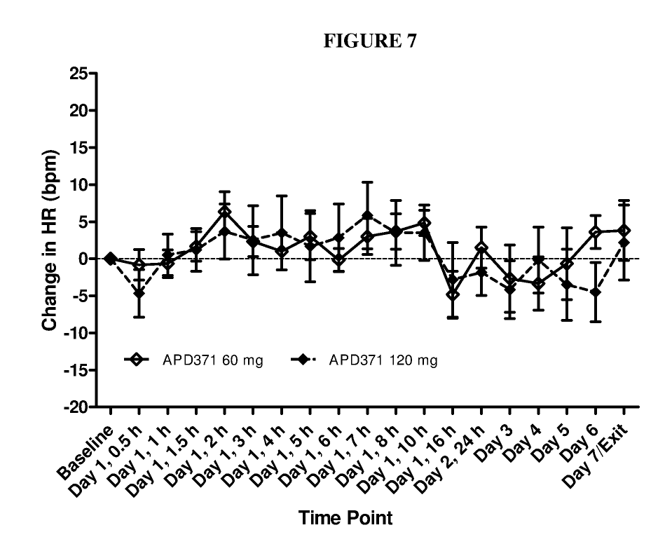

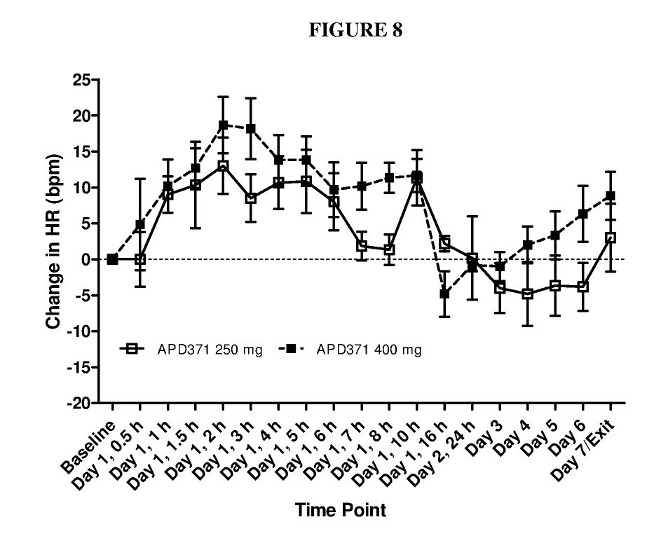

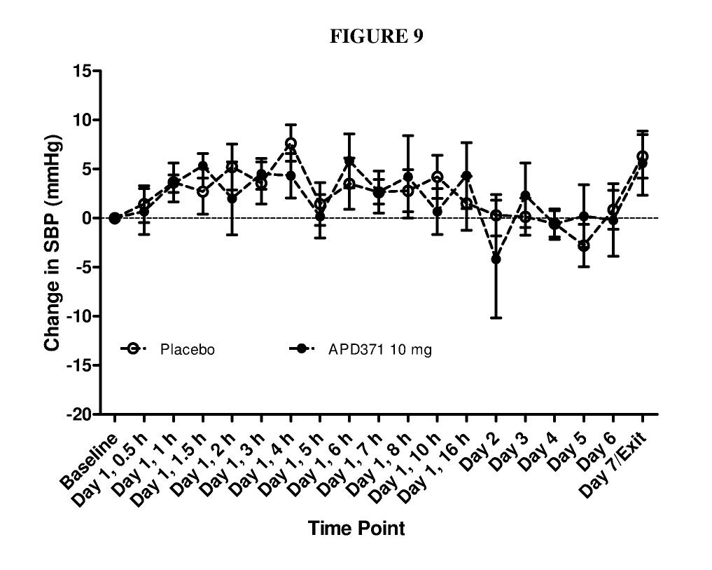

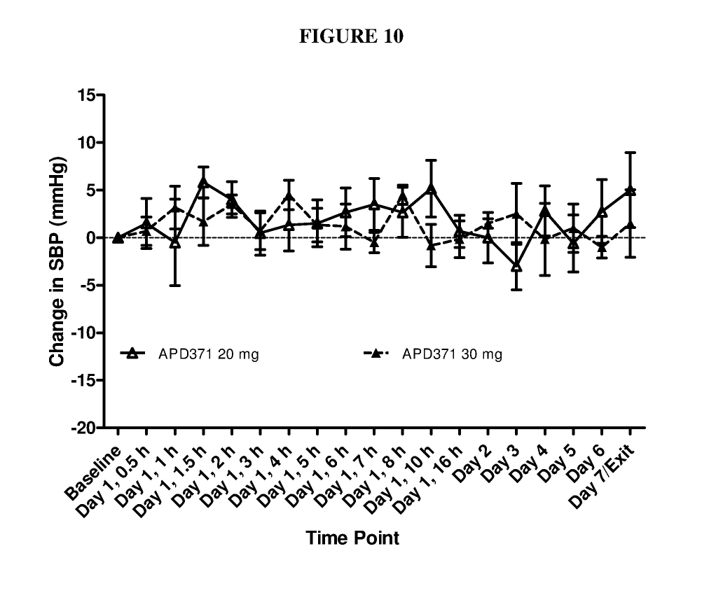

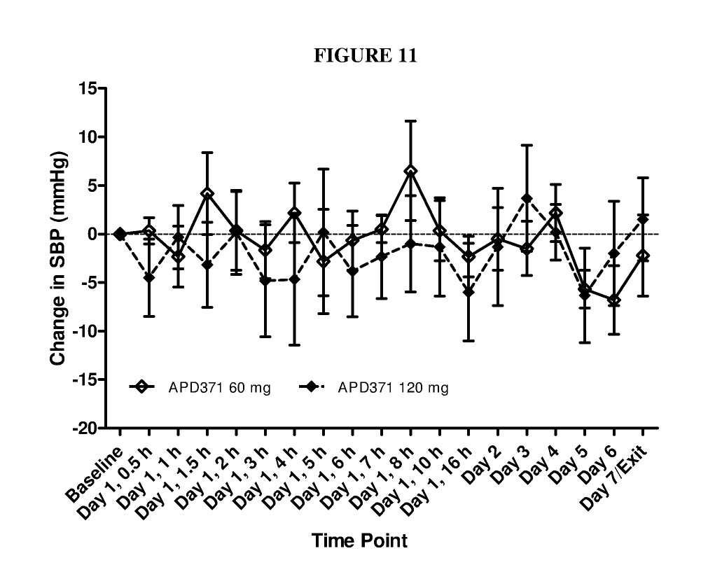

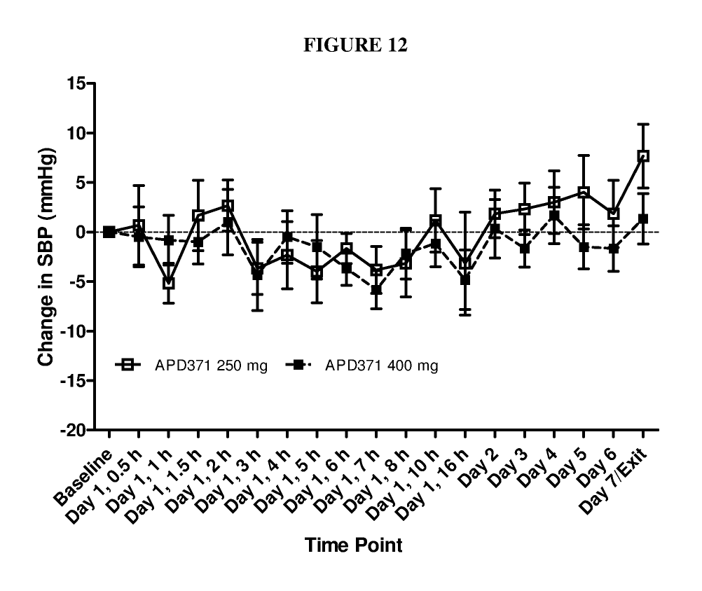

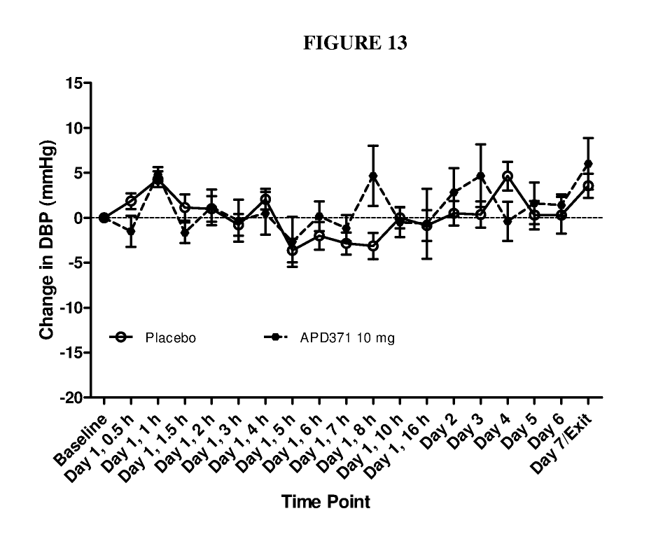

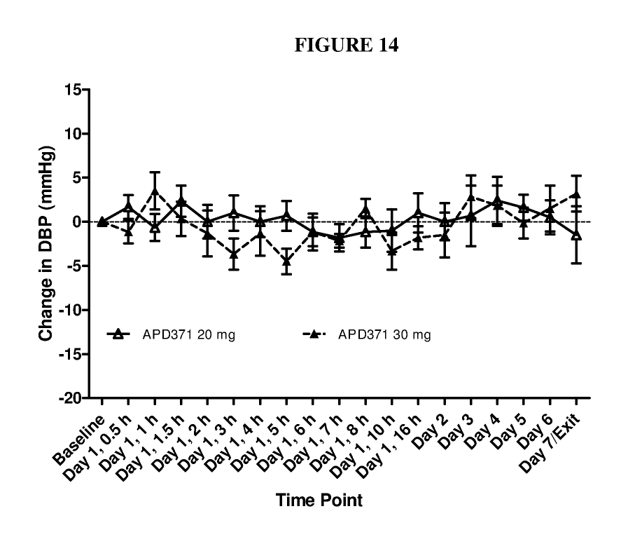

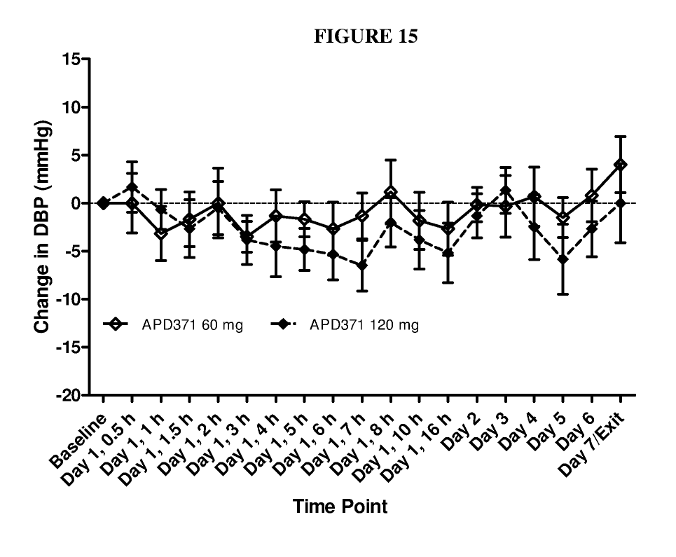

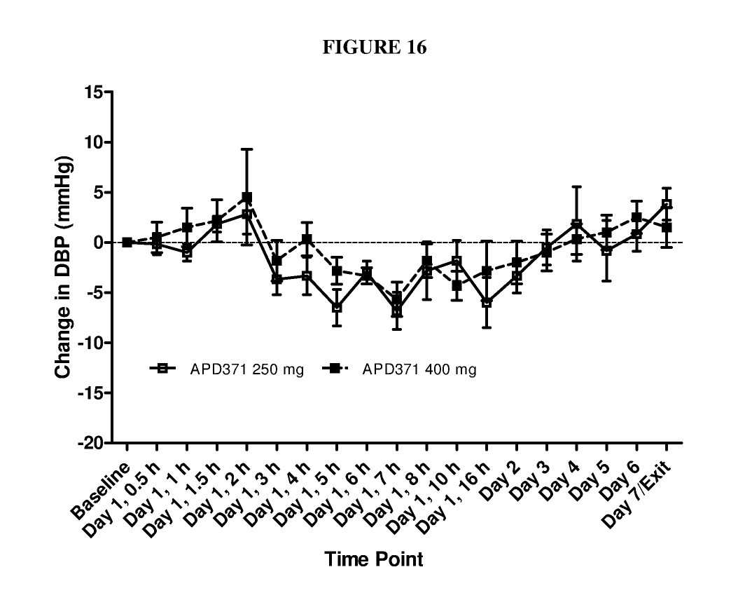

Dose-responsive exposure was observed, and dose escalation was stopped when targeted exposures were reached at 400 mg. All doses of APD371 were well tolerated. However, an asymptomatic mean increase in heart rate was observed at the highest doses compared to baseline (FIGS. 1-8). Variations were also seen for systolic and diastolic blood pressure compared to baseline (FIGS. 9-16).

### Example 2

A phase 1b randomized, double-blind, placebo-controlled multiple ascending dose clinical trial was performed in 36 healthy human subjects to assess the safety, tolerability, and pharmacokinetics of APD371 (Compound B). Doses of 50 mg three times daily (TID), 100 mg TID, and 200 mg TID were administered for 10 days. Subjects were in the clinic for approximately 17 days (referred to as Day −2 to Day 15). Study medication (APD371 or placebo) was administered TID on Days 1-10, and once on Day 11. Each cohort included 12 human subjects, nine of whom were administered APD371 and three of whom were administered placebo.

Vital signs were measured for blood pressure and heart rate in the supine position after resting for five minutes. 12-lead electrocardiograms were used to measure RR, PR, QRS, QT, QTc, QTcB, and QTcF in the recumbent position after resting for 10 minutes. 5-lead electrocardiograms were used for continuous telemetry, with measurements taken during the last five minutes of a resting period of 14 minutes±1 minute in the supine position. Baseline measurements were taken prior to dosing.

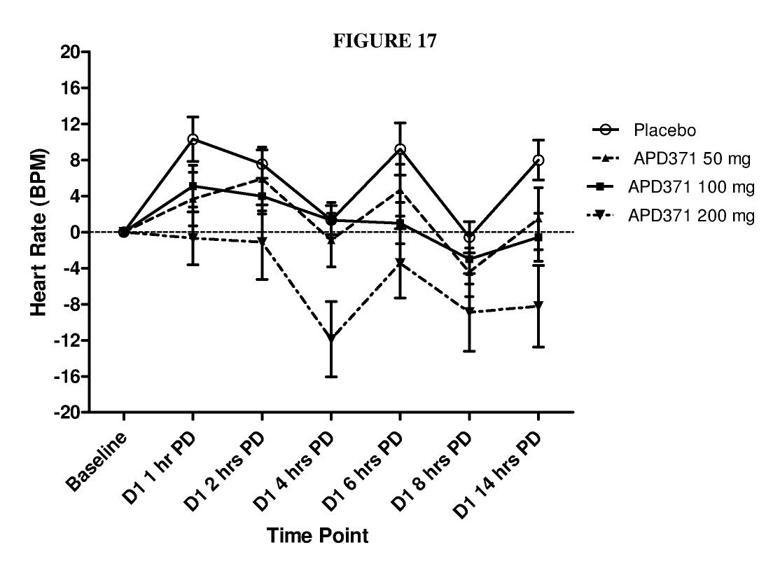

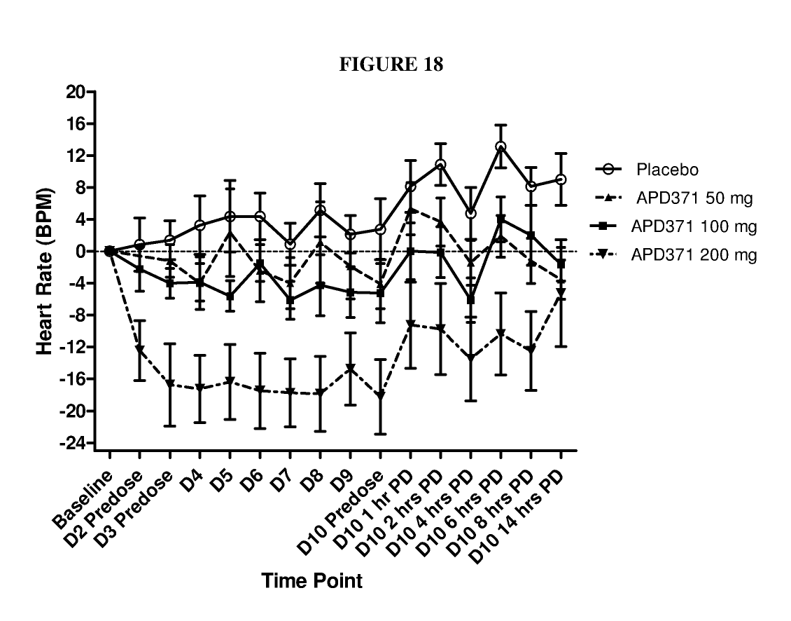

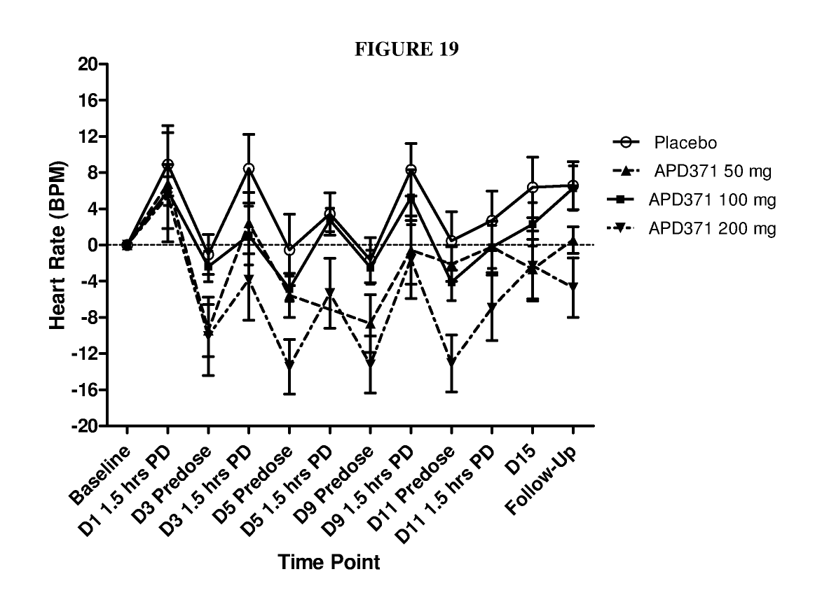

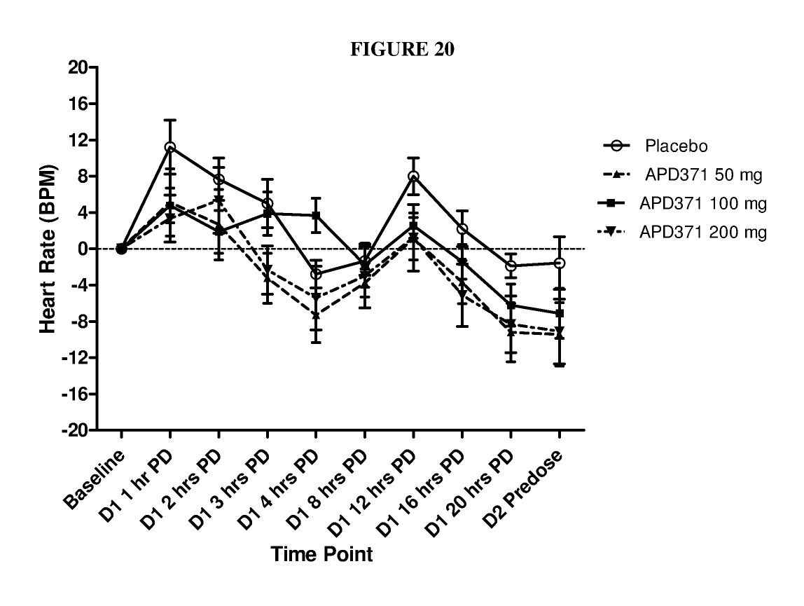

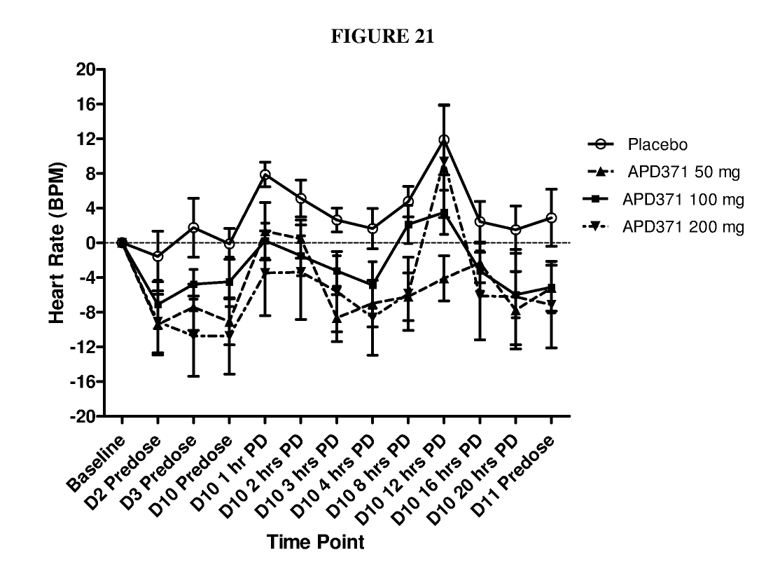

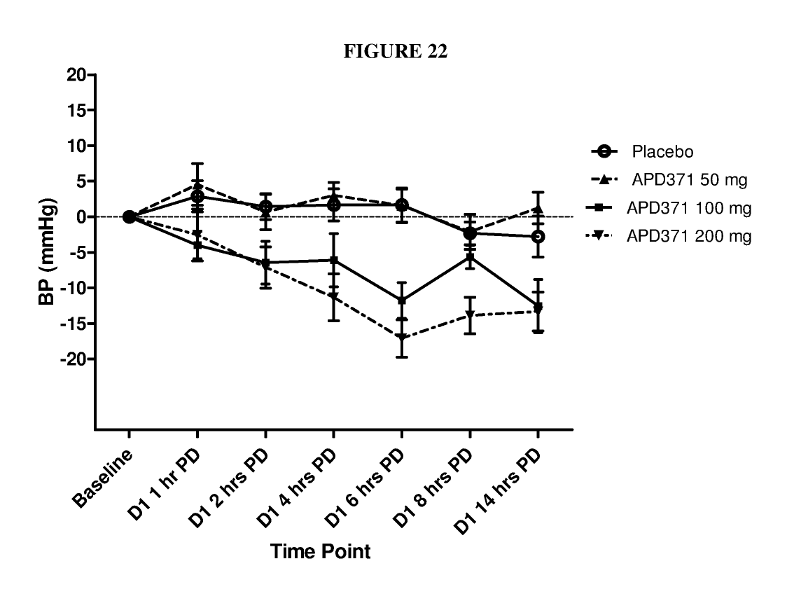

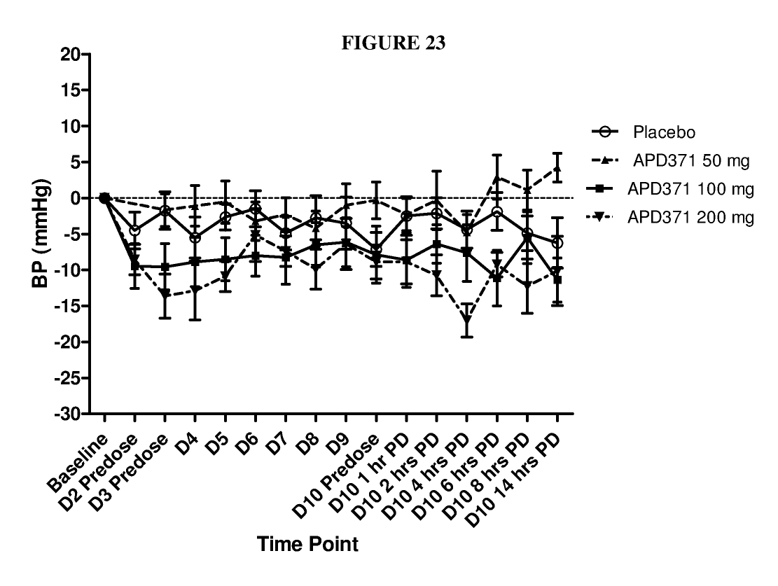

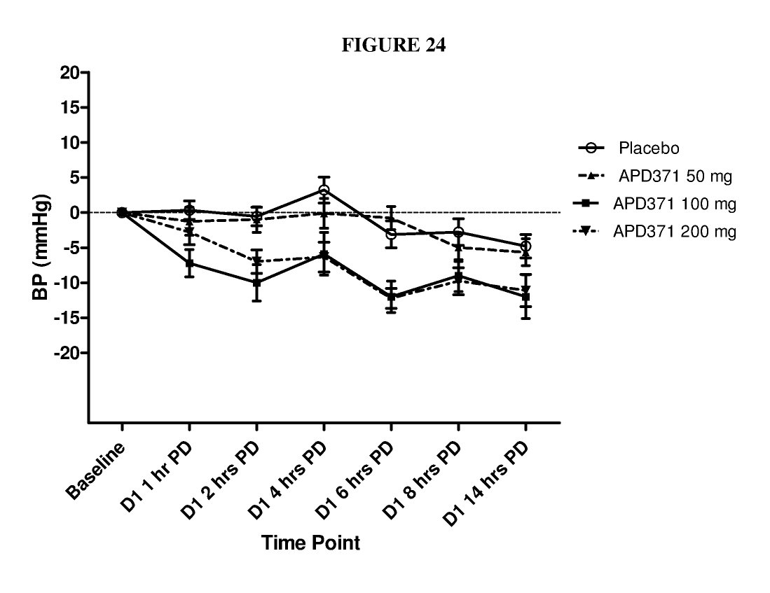

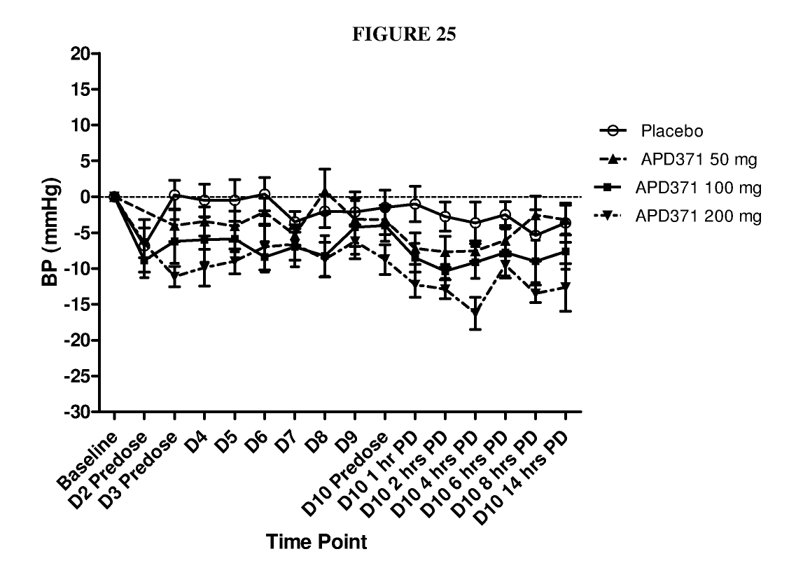

APD371 was well tolerated with multiple dosing. Dose-responsive exposure was observed. Drug levels at all doses were well above those needed to stimulate the CB₂ receptor. The most common adverse events were headache and nausea. All adverse events were classified as mild, and there were no serious adverse events reported. There was one discontinuation in the high-dose group due to an adverse event of mild thirst and somnolence. However, unexpectedly (especially in view of the single ascending dose study), asymptomatic reductions in blood pressure and heart rate were observed (FIGS. 17-25).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a human subject in need of treatment with a selective CB2 receptor agonist, the method comprising the steps of:
a) detecting the presence or absence of a risk factor in the human subject, wherein the risk factor is one or both of:
i. a low heart rate and/or low blood pressure; and
ii. the risk of a low heart rate and/or low blood pressure;
and
b1) administering a therapeutically effective amount of the selective CB2 receptor agonist to the human subject if the risk factor of step a) is absent; or
b2) if the risk factor of step a) is detected, then either:
i. not administering the selective CB2 receptor agonist to the human subject; or
ii. administering the selective CB2 receptor agonist to the human subject at a dose lower than the therapeutically effective amount of step b1).

2. The method of claim 1, wherein the risk factor of step a) is absent.

3. The method of claim 2, further comprising administering to the human subject a further dose of the selective CB2 receptor agonist, wherein the amount of the further dose is the same or greater than the therapeutically effective amount of the selective CB2 receptor agonist of step b1).

4. The method of claim 2, further comprising administering to the human subject a further dose of the selective CB2 receptor agonist, wherein the amount of the further dose is the same as the therapeutically effective amount of the selective CB2 receptor agonist of step b1).

5. The method of claim 1, wherein the risk factor of step a) is detected.

6. The method of claim 5, wherein the selective CB2 receptor agonist is administered to the human subject at a dose lower than the therapeutically effective amount of step b1).

7. The method of claim 6, further comprising administering to the human subject one or more further doses of the selective CB2 receptor agonist, wherein the one or more further doses are at an amount of selective CB2 receptor agonist that is less than the therapeutically effective amount of step b1), and greater than the lower dose of claim 6.

8. The method of claim 7, wherein the one or more further doses of the selective CB2 receptor agonist are of progressively increasing amounts of the selective CB2 receptor agonist.

9. The method of claim 6, further comprising increasing the dosage amount of the selective CB2 receptor agonist.

10. The method of any of claims 1-9, wherein the risk of a low heart rate and/or low blood pressure of step a) is one or more of the conditions listed in paragraphs a-f below:
a. the subject:
i. has had or is taking prolonged bed rest;
ii. is within the first 24 weeks of pregnancy;
iii. has suffered decreases in blood volume (e.g., as a result of trauma, severe internal bleeding, dehydration);
iv. is taking an anti-hypertensive medication, a diuretic, a beta-blocker, a drug for Parkinson's disease, a tricyclic antidepressant, an erectile dysfunction drug alone or in combination with nitroglycerine, digoxin, or an antiarrhythmic;
v. is taking narcotics or alcohol;
vi. has had a heart attack;
vii. has problems with heart valve(s);
viii. has coronary artery disease;
ix. has endocarditis, myocarditis, hypothyroidism, parathyroid disease, Addison's disease, low blood sugar, diabetes, septic shock, neutrally mediated hypotension, anemia, an electrolyte imbalance, high levels of potassium in the blood, or a deficiency in vitamin B-12 and/or folic acid;
b. prior administration of nitrates, alpha blockers, beta blockers, anti-hypertensive drugs, vasodilators, digoxin, amiodarone, alcohol, or medications that are inhibitors or inducers of CYP, for example CYP1A2, CYP2B6, CYP2C8, CYP2C9, CYP2C19, CYP2D6, CYP2E1, CYP3A or CYP3A4/3A5;
c. prior administration of one or more:
i. agents that increase the exposure of the selective CB2 receptor agonist;
ii. agents that slow the metabolism of the selective CB2 receptor agonist;
iii. agents that increase the accumulation of metabolites of the selective CB2 receptor agonist in the human subject compared to the absence of the drug or agent;
iv. agents that compete with the selective CB2 receptor agonist for protein binding; or
v. agents that cause QT prolongation;
d. a medical condition that is known to decrease heart rate and/or blood pressure, for example a heart condition that leads to low heart rate (bradycardia);
e. a history of cerebrovascular disease, dizziness, lightheadedness, fainting, headache, nausea, hypotension, syncope, shock, hemodynamic instability, bradycardia, aortic stenosis, myocardial infarction, ischemia, heart failure, or a conduction abnormality; and
f. impaired function of a CYP, or being a poor or intermediate CYP metabolizer, for example where the CYP is CYP1A2, CYP2B6, CYP2C8, CYP2C9, CYP2C19, CYP2D6, CYP2E1, CYP3A or CYP3A4/3A5.

11. The method of any of claims 1-10, wherein the risk factor of step a) is a low heart rate and/or low blood pressure.

12. The method of any of claims 1-10, wherein the risk factor of step a) is the risk of a low heart rate and/or low blood pressure.

13. A method of treating a human subject in need of treatment with a selective CB2 receptor agonist, the method comprising the steps of:
a. administering a therapeutically effective amount of the selective CB2 receptor agonist to the human subject;
b. detecting the heart rate and/or blood pressure of the human subject after administration of the selective CB2 receptor agonist; and either:
i. continuing administration of the selective CB2 receptor agonist to the human subject if the heart rate and/or blood pressure of the human subject after administration of the selective CB2 receptor agonist is not decreased by a defined amount compared to the heart rate and/or blood pressure prior to administration of the selective CB2 receptor agonist; or
ii. discontinuing the administration of the therapeutically effective amount of the selective CB2 receptor agonist if the heart rate and/or blood pressure of the human subject after administration of the selective CB2 receptor agonist is decreased by a defined amount compared to the heart rate and/or blood pressure prior to administration of the selective CB2 receptor agonist.

14. The method of claim 13, wherein the heart rate and/or blood pressure of the subject after administration of the selective CB2 receptor agonist is decreased by a defined amount compared to the heart rate and/or blood pressure prior to administration of the selective CB2 receptor agonist;
the method further comprising administering to the human subject a lower dose of the selective CB2 receptor agonist.

15. The method of claim 14, wherein the lower dose selective CB2 receptor agonist is less than the therapeutically effective amount of the selective CB2 receptor agonist of step (a).

16. The method of claim 15, further comprising administering to the human subject one or more further doses of the selective CB2 receptor agonist, wherein the one or more further doses are at an amount of selective CB2 receptor agonist that is less than the therapeutically effective amount of step (a), and greater than the lower dose of claim 15.

17. The method of claim 16, wherein the one or more further doses of the selective CB2 receptor agonist are of progressively increasing amounts of the selective CB2 receptor agonist.

18. The method of claim 14, further comprising increasing the dosage amount of the selective CB2 receptor agonist.

19. The method of claim 13, wherein the heart rate and/or blood pressure of the subject after administration of the selective CB2 receptor agonist is not decreased by a defined amount compared to the heart rate and/or blood pressure prior to administration of the selective CB2 receptor agonist;
the method further comprising administering to the human subject a further dose of the selective CB2 receptor agonist that is the same or greater than the therapeutically effective amount of the selective CB2 receptor agonist of step a).

20. The method of claim 19, wherein the further dose of the selective CB2 receptor agonist is the same as the therapeutically effective amount of the selective CB2 receptor agonist of step a).

21. The method of claim 19, wherein the further dose of the selective CB2 receptor agonist is greater than the therapeutically effective amount of the selective CB2 receptor agonist of step a).

22. A method of treating a human subject in need of treatment with a selective CB2 receptor agonist, the method comprising the steps of:
a) detecting the heart rate and/or blood pressure of the human subject prior to administration of a selective CB2 receptor agonist;
b) administering a therapeutically effective amount of the selective CB2 receptor agonist to the human subject;
c) detecting the heart rate and/or blood pressure of the human subject after administration of the selective CB2 receptor agonist; and either:
i) if the heart rate and/or blood pressure detected in step c) are not decreased by a defined amount compared to the heart rate and/or blood pressure prior to administration of the selective CB2 receptor agonist, then continuing administering the selective CB2 receptor agonist to the human subject; or
ii) if the heart rate and/or blood pressure detected in step c) is decreased by a defined amount compared to the heart rate and/or blood pressure prior to administration of the selective CB2 receptor agonist, then either:
discontinuing administering the selective CB2 receptor agonist to the human subject; or
continuing administering the selective CB2 receptor agonist to the human subject at a dose lower than the dose of the selective CB2 receptor agonist of step b).

23. The method of claim 22, wherein the heart rate and/or blood pressure detected in step c) are not decreased by a defined amount compared to the heart rate and/or blood pressure prior to administration of the selective CB2 receptor agonist.

24. The method of claim 22, wherein the heart rate and/or blood pressure detected in step c) is decreased by a defined amount compared to the heart rate and/or blood pressure prior to administration of the selective CB2 receptor agonist.

25. The method of claim 24, wherein administration of the selective CB2 receptor agonist to the human subject is continued at a dose lower than the dose of the selective CB2 receptor agonist of step b).

26. The method of claim 25, further comprising administering to the human subject one or more further doses of the selective CB2 receptor agonist, wherein the one or more further doses are at an amount of selective CB2 receptor agonist that is less than the therapeutically effective amount of step b), and greater than the lower dose of step (c) ii.

27. The method of claim 26, wherein the one or more further doses of the selective CB2 receptor agonist are of progressively increasing amounts of the selective CB2 receptor agonist.

28. The method of claim 22, further comprising increasing the dosage amount of the selective CB2 receptor agonist.

29. The method of claim any of claims 18-24, wherein the dose lower than the dose of the selective CB2 receptor agonist of step b) is:
a dose that is from, or from about, 10% to 80% of the therapeutically effective amount of the selective CB2 receptor agonist of step b), for example 80%, 75%, 70%, 65%, 60%, 55%, 50%, 45%, 40%, 35%, 30%, 25%, 20%, 15%, or 10% of the therapeutically effective amount of the selective CB2 receptor agonist of step b); for example 50% or 75% of the therapeutically effective amount of the selective CB2 receptor agonist of step b); or
a dose that is selected from, or from about: 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, and 475 mg; for example 25 mg, 50 mg, 75 mg, or 100 mg three times daily; for example 75 mg, 150 mg; 225 mg or 300 mg daily;

30. The method of any of claims 13-25 wherein the human subject was previously administered an agent selected from one or more of the agents listed below in paragraphs a-c:
a. an anti-hypertensive medication, a diuretic, a beta-blocker, a drug for Parkinson's disease, a tricyclic antidepressant, an erectile dysfunction drug alone or in combination with nitroglycerine, digoxin, or an antiarrhythmics, narcotics, or alcohol;
b. nitrates, alpha blockers, beta blockers, anti-hypertensive drugs, vasodilators, digoxin, amiodarone, alcohol, or medications that are inhibitors or inducers of CYP, for example CYP1A2, CYP2B6, CYP2C8, CYP2C9, CYP2C19, CYP2D6, CYP2E1, CYP3A, or CYP3A4/3A5; and
c. agents that:
i. increase the exposure of the selective CB2 receptor agonist;
ii. slow the metabolism of the selective CB2 receptor agonist;
iii. increase the accumulation of metabolites of the selective CB2 receptor agonist in the human subject compared to the absence of the drug or agent;
iv. compete with the selective CB2 receptor agonist for protein binding; or
v. cause QT prolongation.

31. The method of any one of the preceding claims, further comprising the step of identifying a human subject in need of treatment with a selective CB2 receptor agonist.

32. The method of any one of the preceding claims, wherein the low heart rate is less than, or less than about, 60, 55, 50, 45, or 40 beats per minute (bpm); for example less than, or less than about, 50 bpm; and/or wherein the low heart rate is at least, or at least about, a 10 bpm reduction from the heart rate prior to administration of the selective CB2 receptor agonist.

33. The method of any one of the preceding claims, wherein the low blood pressure is a systolic blood pressure of less than, or less than about, 120, 115, 110, 105, 100, 95, 90, 85, 80, 75, or 70 mmHg; for example a systolic blood pressure less than, or less than about, 90 mmHg.

34. The method of any one of the preceding claims, wherein the low blood pressure is a diastolic blood pressure of less than, or less than about, 80, 75, 70, 65, 60, 55, or 50 mmHg; for example a diastolic blood pressure less than, or less than about, 60 mmHg; or a diastolic blood pressure less than, or less than about, 50 mmHg.

35. The method of any one of the preceding claims, wherein the low blood pressure is a systolic blood pressure less than, or less than about 90 mmHg, and wherein the diastolic blood pressure less than, or less than about, 50 mmHg, or a systolic blood pressure less than, or less than about 95 mmHg, and wherein the diastolic blood pressure less than, or less than about, 60 mmHg, and wherein the decrease by a defined amount of blood pressure decrease is at least a 10 mmHg reduction from the systolic and/or diastolic blood pressure prior to administration of the selective CB2 receptor agonist.

36. The method of any one of the preceding claims, wherein the decrease in heart rate by a defined amount is a decrease in heart rate of at least, or of at least about, 5%, 10%, 15%, 20%, or 25%; or of, or of about, 5, 10, 15, 20, or 25 bpm.

37. The method of any one of the preceding claims, wherein the decrease in blood pressure by a defined amount is a decrease from baseline systolic blood pressure of at least, or at least about, 5%, 10%, 15%, 20%, or 25%; and/or a decrease from baseline diastolic blood pressure of at least, or at least about, 5%, 10%, 15%, 20%, or 25%.

38. The method of any one of the preceding claims, wherein the decrease in blood pressure by a defined amount is a decrease in systolic blood pressure of at least, or of at least about, 5, 10, 15, 20, or 25 mmHg; and/or a decrease in diastolic blood pressure of at least, or of at least about, 5, 10, 15, 20, or 25 mmHg.

39. The method of any one of the preceding claims, wherein the selective CB2 receptor agonist is APD371, and wherein the therapeutically effective amount of the selective CB2 receptor agonist is selected from, or from about: 10 mg to 500 mg; for example 25 mg to 250 mg; for example 25 mg, 50 mg, 75 mg, 100 mg, 200 mg, or 250 mg.

40. The method of any one of the preceding claims, wherein the therapeutically effective amount of the selective CB2 receptor agonist is administered more than once; for example at a frequency of: once a day, twice a day, three times a day, or four times a day.

41. The method of any one of the preceding claims, wherein the selective CB2 receptor agonist is APD371, and wherein the further dosage amount is selected from, or from about: 10 mg to 500 mg; for example 25 mg to 250 mg; for example 25 mg, 50 mg, 75 mg, 100 mg, 200 mg, or 250 mg.

42. The method of any one of the preceding claims, wherein the further dosage amount of the selective CB2 receptor agonist is administered more than once; for example at a frequency selected of: once a day, twice a day, three times a day, or four times a day.

43. The method of any one of the preceding claims, further comprising evaluating the heart rate and/or blood pressure of the human subject following administration of the further dose.

44. The method of any one of the preceding claims, wherein the selective CB2 receptor agonist is APD371, and wherein the therapeutically effective amount of the selective CB2 receptor agonist is a dose of, or about, 25 mg to 100 mg.

45. The method of claim 32, wherein the therapeutically effective amount of the selective CB2 receptor agonist is from 25 mg to 100 mg per administration, administered twice or three times daily.

46. The method of claim 41, wherein the therapeutically effective amount of the selective CB2 receptor agonist is less than about 600, about 400, about 300, or about 250 mg daily.

47. The method of claim 41, wherein the therapeutically effective amount of the selective CB2 receptor agonist is about 75 mg, about 150 mg, about 225 mg, or about 300 mg daily.

48. The method of any one of the preceding claims, wherein the dose lower than the therapeutically effective amount of the selective CB2 receptor agonist is, or is about, 50 mg.

49. The method of any one of the preceding claims, wherein the dose lower than the therapeutically effective amount of the selective CB2 receptor agonist is, or is about, 100 mg.

50. The method of any one of the preceding claims, wherein the dose lower than the therapeutically effective amount of the selective CB2 receptor agonist is administered at a frequency selected from the group consisting of: once a day, twice a day, three times a day, and four times a day.

51. The method of any one of the preceding claims, further comprising evaluating the heart rate and/or blood pressure of the human subject following administration of the lower dosage amount.

52. The method of any one of the preceding claims, further comprising monitoring the human subject for an adverse reaction following administration of the selective CB2 receptor agonist.

53. The method of any one of the preceding claims, further comprising evaluating the heart rate and/or blood pressure and/or a condition related thereto for the human subject following administration of the selective CB2 receptor agonist; for example wherein evaluating the heart rate and/or blood pressure of the human subject comprises measuring the heart rate and/or blood pressure of the human subject.

54. The method of claim 49, wherein the heart rate and/or blood pressure of the human subject is evaluated about, or at least about, 0.5, 1, 1.5, 2, 2.5, 3, 3.5, 4, 4.5, 5, 5.5, or 6 hours following administration of the selective CB2 receptor agonist.

55. The method of any one of the preceding claims, wherein a risk factor is detected or not detected, or the heart rate and/or blood pressure of the human subject is detected and/or evaluated, based on at least one determination selected from the group consisting of:
(i) determining by electrocardiogram that the human subject has or does not have a low heart rate;
(ii) determining by vital signs that the human subject has or does not have a low heart rate;
(iii) determining by vital signs that the human subject has or does not have a low systolic and/or diastolic blood pressure;
(iv) determining that the human subject has or does not have a history of low heart rate and/or low systolic and/or diastolic blood pressure and/or condition related thereto;
(v) determining that the human subject has or does not have impaired elimination of the selective CB2 receptor agonist; and
(vi) determining that the human subject is or is not a poor or intermediate CYP metabolizer.

56. The method of any one of the preceding claims, wherein the selective CB2 receptor agonist is APD371, wherein the therapeutically effective amount of APD371 is, or is about:
10 mg, 15 mg, 20 mg, 25 mg, 30 mg, 35 mg, 40 mg, 45 mg, 50 mg, 60 mg, 65 mg, 70 mg, 75 mg, 80 mg, 85 mg, 90 mg, 95 mg, 100 mg, 110 mg, 120 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg; or is, or is about, 10 mg, 25 mg, 50 mg, 75 mg, 150 mg, or 200 mg; and is administered once, twice, three times or four times per day.

57. The method of any one of the preceding claims, wherein the selective CB2 receptor agonist is APD371, wherein the maximum dose of APD371 is, or is about:
10 mg, 25 mg, 50 mg, 75 mg, 100 mg, 125 mg, 150 mg, 175 mg, or 200 mg per administration; and/or
75 mg, 150 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, or 600 mg per day.

58. The method of any one of the preceding claims, wherein the selective CB2 receptor agonist is APD371, and wherein the amount of APD371 is less than or equal to 200 mg per administration.

59. The method of any one of the preceding claims, wherein the human subject is not part of a multi-center, placebo-controlled, double-blind trial designed to:
(ii) examine the safety or efficacy of the selective CB2 receptor agonist, and/or
(iii) have data therefrom submitted to a regulatory agency for approval of the selective CB2 receptor agonist for treatment of human subjects.

60. The method of any one of the preceding claims, wherein the human subject is elderly.

61. The method of any one of the preceding claims, wherein:
the heart rate is selected from the group consisting of: a resting heart rate, a supine heart rate, and a standing heart rate;
the blood pressure is selected from the group consisting of systolic blood pressure and diastolic blood pressure; wherein:
the systolic blood pressure is selected from the group consisting of a resting systolic blood pressure, a supine systolic blood pressure, and a standing systolic blood pressure; and
the diastolic blood pressure is selected from the group consisting of a resting diastolic blood pressure, a supine diastolic blood pressure, and a standing diastolic blood pressure.

62. The method of any one of the preceding claims, wherein the selective CB2 receptor agonist is administered orally.

63. The method of any one of the preceding claims, wherein the selective CB2 receptor agonist is in the form of a tablet or capsule.

64. The method of any one of the preceding claims, wherein the treatment is the treatment or prevention of a CB2 receptor-mediated disorder.

65. The method of any one of the preceding claims, wherein the treatment is the treatment or prevention of a CB2 receptor-mediated disorder selected from the group consisting of: pain associated with osteoarthritis, chemotherapy-induced pain, neuropathic pain, acute post-operative pain, abdominal pain associated with inflammatory bowel disease (IBD), non-radicular low back pain, liver fibrosis, primary biliary cirrhosis, nonalcoholic steatohepatitis, renal fibrosis, osteoarthritis, endometriosis, interstitial cystitis, and migraine.

66. The method of any one of the preceding claims, wherein the treatment is the treatment of acute and/or chronic inflammatory pain.

67. The method of any one of the preceding claims, wherein the treatment is the treatment of acute and/or chronic neuropathic pain.

68. The method of any one of the preceding claims, wherein the human subject is a poor or intermediate CYP metabolizer.

69. The method of any one of the preceding claims, wherein the human subject is a poor or intermediate CYP metabolizer, and wherein the CYP is selected from the group consisting of: CYP1A2, CYP2B6, CYP2C8, CYP2C9, CYP2C19, CYP2D6, CYP2E1, CYP3A, and CYP3A4/3A5.

70. The method of any one of the preceding claims, further comprising determining that the human subject is stable on alpha-blocker therapy prior to initiating treatment with the selective CB2 receptor agonist.

71. The method of any one of the preceding claims, wherein the selective CB2 receptor agonist increases internalization of the CB2 receptor in a cell to at least 75%, at least 80%, at least 85%, at least 90%, at least 95%, at least 96%, at least 97%, at least 98%, or at least about 99% the level of internalization that would occur if the cell were contacted with CP55,940.

72. The method of any one of the preceding claims, wherein the selectivity of the selective CB2 receptor agonist is, or has previously been identified as being, at least 50-fold, at least 100-fold, at least 500-fold, at least 750-fold, at least 1000-fold, at least 2000-fold, at least 3000-fold, at least 4000-fold, at least 5000-fold, at least 6000-fold, at least 7000-fold, at least 8000-fold, at least 9000-fold, or at least 10,000-fold selectivity for the human CB2 receptor relative to the human CB1 receptor.

73. A selective CB2 receptor agonist for use in the treatment of pain in a human subject in need of such treatment, wherein the heart rate and/or blood pressure of the subject after administration of the selective CB2 receptor agonist is not decreased by a defined amount compared to the heart rate and/or blood pressure prior to administration of the selective CB2 receptor agonist.

74. A selective CB2 receptor agonist for use in the treatment of pain in a human subject in need of such treatment, wherein the heart rate and/or blood pressure of the subject after administration of the selective CB2 receptor agonist is decreased by a defined amount compared to the heart rate and/or blood pressure prior to administration of the selective CB2 receptor agonist.

75. A selective CB2 receptor agonist for use in the treatment of pain in a human subject in need of such treatment, wherein human subject does not develop bradycardia after administration of the selective CB2 receptor agonist.

76. The selective CB2 receptor agonist of any of claims 63-65, wherein the pain is:
bone pain; joint pain; muscle pain; dental pain; migraine and other headache pain; inflammatory pain including acute inflammatory pain and chronic inflammatory pain; acute and/or chronic neuropathic pain; pain that occurs as an adverse effect of therapeutics;
pain associated with a disorder selected from: osteoarthritis, cancer, multiple sclerosis, allergic reactions, nephritic syndrome, scleroderma, thyroiditis, diabetic neuropathy, fibromyalgia, HIV related-neuropathy, neuralgias, sciatica, and autoimmune conditions;
chemotherapy-induced pain; acute post-operative pain; abdominal pain associated with inflammatory bowel disease (IBD); non-radicular low back pain; pain from liver fibrosis, primary biliary cirrhosis, nonalcoholic steatohepatitis, renal fibrosis, endometriosis, and interstitial cystitis; hyperalgesia; allodynia; inflammatory hyperalgesia; neuropathic hyperalgesia; acute nociception; osteoporosis; and multiple sclerosis-associated spasticity.

77. The method of any one of the preceding claims, wherein the selective CB2 receptor agonist is APD371.
